(19) United States
(12) Patent Application Publication
(10) Pub. No.: US 2016/0272635 A1
(43) Pub. Date: Sep. 22, 2016

(54) SUBSTITUTED DIHYDROPYRIDO[3,4-B]PYRAZINONES AS DUAL INHIBITORS OF BET PROTEINS AND POLO-LIKE KINASES

(71) Applicant: BAYER PHARMA AKTIENGESELLSCHAFT, Berlin (DE)

(72) Inventors: Norbert Schmees, Berlin (DE); Benjamin Bader, Berlin (DE); Bernard Haendler, Berlin (DE); Volker Schulze, Hohen Neuendorf, OT Bergf (DE); Ingo Hartung, Berlin (DE); Niels Bohnke, Berlin (DE); Florian Puehler, Massachusetts (DE)

(21) Appl. No.: 14/907,367

(22) PCT Filed: Jul. 21, 2014
(86) PCT No.: PCT/EP2014/065605

§ 371 Date: Jan. 25, 2016

(30) Foreign Application Priority Data
Jul. 23, 2013 (EP) 13177539.7

(51) Int. Cl. C07D 471/04; A61K 31/55; A61K 31/4985

(52) U.S. Cl. CPC C07D 471/04; A61K 31/4985; A61K 31/55

(57) ABSTRACT

The present invention relates to substituted dihydropyrido[3,4-b]pyrazinones as dual inhibitors of BET proteins, in particular BRD4 proteins, and Polo-like kinases, in particular Plk-1 proteins of the general formula (I)

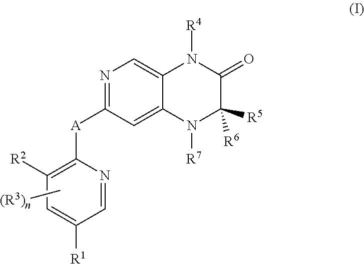

in which A, X, R¹, R², R³, R⁴, R⁵, R⁶, R⁷ and n are each as defined in the description, to intermediates for preparation of the compounds according to the invention, to pharmaceutical compositions comprising the compounds according to the invention, and to the prophylactic and therapeutic use thereof in the case of hyperproliferative disorders, especially in the case of tumour disorders.

Furthermore, the present invention relates to the use of the dihydropyrido[3,4-b]pyrazinones according to the invention in viral infections, in neurodegenerative disorders, in inflammation disorders, in atherosclerotic disorders and in male fertility control.

The present invention relates to substituted dihydropyrido[3,4-b]pyrazinones as dual inhibitors of BET proteins, in particular BRD4 proteins, and Polo-like kinases, in particular Plk-1 proteins, to intermediates for preparing the compounds according to the invention, to pharmaceutical compositions comprising the compounds according to the invention and to their prophylactic and therapeutic use for hyperproliferative disorders, in particular for tumour disorders. Furthermore, the present invention relates to the use of the dihydropyrido[3,4-b]pyrazinones according to the invention in viral infections, in neurodegenerative disorders, in inflammatory diseases, in atherosclerotic disorders and in male fertility control.

The human BET family (bromo domain and extra C-terminal domain family) has four members (BRD2, BRD3, BRD4 and BRDT) containing two related bromo domains and one extraterminal domain (Wu and Chiang, J. Biol. Chem., 2007, 282:13141-13145). The bromo domains are protein regions which recognize acetylated lysine residues. Such acetylated lysines are often found at the N-terminal end of histones (e.g. histone 3 or histone 4), and they are features of an open chromatin structure and active gene transcription (Kuo and Allis, Bioessays, 1998, 20:615-626). The different acetylation patterns recognized by BET proteins in histones were investigated in depth (Umehara et al., J. Biol. Chem., 2010, 285:7610-7618; Filippakopoulos et al., Cell, 2012, 149:214-231). In addition, bromo domains can recognize further acetylated proteins. For example, BRD4 binds to RelA, which leads to stimulation of NF-KB and transcriptional activity of inflammatory genes (Huang et al., Mol. Cell. Biol., 2009, 29:1375-1387; Zhang et al., J. Biol. Chem., 2012, 287: 28840-28851; Zou et al., Oncogene, 2013, doi:10.1038/onc.2013.179). BRD4 also binds to cyclin T1 and forms an active complex which is important for transcription elongation (Schroder et al., J. Biol. Chem., 2012, 287:1090-1099). The extraterminal domain of BRD2, BRD3 and BRD4 interacts with several proteins involved in chromatin modulation and the regulation of gene expression (Rahman et al., Mol. Cell. Biol., 2011, 31:2641-2652).

In mechanistic terms, BET proteins play an important role in cell growth and in the cell cycle. They are associated with mitotic chromosomes, which suggests a role in epigenetic memory (Dey et al., Mol. Biol. Cell, 2009, 20:4899-4909; Yang et al., Mol. Cell. Biol., 2008, 28:967-976). Involvement of BRD4 in the post-mitotic reactivation of gene transcription has been demonstrated (Zhao et al., Nat. Cell. Biol., 2011, 13:1295-1304). BRD4 is essential for transcription elongation and recruits the elongation complex P-TEFb consisting of CDK9 and cyclin T1, which leads to activation of RNA polymerase II (Yang et al., Mol. Cell, 2005, 19:535-545; Schroder et al., J. Biol. Chem., 2012, 287:1090-1099). Consequently, the expression of genes involved in cell proliferation is stimulated, for example of c-Myc, cyclin D1 and aurora B (You et al., Mol. Cell. Biol., 2009, 29:5094-5103; Zuber et al., Nature, 2011, doi:10.1038). BRD2 is involved in the regulation of target genes of the androgen receptor (Draker et al., PLOS Genetics, 2012, 8, e1003047). BRD2 and BRD3 bind to transcribed genes in hyperacetylated chromatin regions and promote transcription by RNA polymerase II (LeRoy et al., Mol. Cell, 2008, 30:51-60).

The knockdown of BRD4 or the inhibition of the interaction with acetylated histones in various cell lines leads to a G1 arrest (Mochizuki et al., J. Biol. Chem., 2008, 283:9040-9048; Mertz et al., Proc. Natl. Acad. Sci. USA, 2011, 108:16669-16674). It has also been shown that BRD4 binds to promoter regions of several genes which are activated in the G1 phase, for example cyclin D1 and D2 (Mochizuki et al., J. Biol. Chem., 2008, 283:9040-9048). In addition, inhibition of the expression of c-Myc, an essential factor in cell proliferation, after BRD4 inhibition has been demonstrated (Dawson et al., Nature, 2011, 478:529-533; Delmore et al., Cell, 2011, 146:1-14; Mertz et al., Proc. Natl. Acad. Sci. USA, 2011, 108:16669-16674) Inhibition of the expression of androgen-regulated genes and binding of BRD2 to corresponding regulatory regions has also been demonstrated (Draker et al., PLOS Genetics, 2012, 8, e1003047).

BRD2 and BRD4 knockout mice die at an early stage during embryogenesis (Gyuris et al., Biochim. Biophys. Acta, 2009, 1789:413-421; Houzelstein et al., Mol. Cell. Biol., 2002, 22:3794-3802). Heterozygotic BRD4 mice have various growth defects attributable to reduced cell proliferation (Houzelstein et al., Mol. Cell. Biol., 2002, 22:3794-3802).

BET proteins play an important role in various tumour types. Fusion between the BET proteins BRD3 or BRD4 and NUT, a protein which is normally expressed only in the testes, leads to an aggressive form of squamous cell carcinoma, called NUT midline carcinoma (French, Cancer Genet. Cytogenet., 2010, 203:16-20). The fusion protein prevents cell differentiation and promotes proliferation (Yan et al., J. Biol. Chem., 2011, 286:27663-27675, Grayson et al., 2013, doi:10-1038/onc.2013.126). The growth of in vivo models derived therefrom is inhibited by a BRD4 inhibitor (Filippakopoulos et al., Nature, 2010, 468:1067-1073). Screening for therapeutic targets in an acute myeloid leukaemia cell line (AML) showed that BRD4 plays an important role in this tumour (Zuber et al., Nature, 2011, 478, 524-528). Reduction in BRD4 expression leads to a selective arrest of the cell cycle and to apoptosis. Treatment with a BRD4 inhibitor prevents the proliferation of an AML xenograft in vivo. Further experiments with a BRD4 inhibitor show that BRD4 is involved in various haematological tumours, for example multiple myeloma (Delmore et al., Cell, 2011, 146, 904-917) and Burkitt's lymphoma (Mertz et al., Proc. Natl. Acad. Sci. USA, 2011, 108, 16669-16674). In solid tumours too, for example lung cancer, BRD4 plays an important role (Lockwood et al., Proc. Natl. Acad. Sci. USA, 2012, 109, 19408-19413). Elevated expression of BRD4 has been detected in multiple myeloma, and amplification of the BRD4 gene has also been found in patients having multiple myeloma (Delmore et al., Cell, 2011, 146, 904-917). Amplification of the DNA region containing the BRD4 gene was detected in primary breast tumours (Kadota et al., Cancer Res, 2009, 69:7357-7365). For BRD2 too, there are data relating to a role in tumours. A transgenic mouse which overexpresses BRD2 selectively in B cells develops B cell lymphomas and leukaemias (Greenwall et al., Blood, 2005, 103:1475-1484).

BET proteins are also involved in viral infections. BRD4 binds to the E2 protein of various papillomaviruses and is important for the survival of the viruses in latently infected cells (Wu et al., Genes Dev., 2006, 20:2383-2396; Vosa et al., J. Virol., 2006, 80:8909-8919). The herpes virus, which is responsible for Kaposi's sarcoma, also interacts with various BET proteins, which is important for disease survival (Viejo-Borbolla et al., J. Virol., 2005, 79:13618-13629; You et al., J. Virol., 2006, 80:8909-8919). Through binding to P-TEFb, BRD4 also plays an important role in the replication of HIV-1 (Bisgrove et al., Proc. Natl Acad. Sci. USA, 2007, 104:13690-13695). Treatment with a BRD4 inhibitor leads to stimulation of the dormant, untreatable reservoir of HIV-1 viruses in T cells (Banerjee et al., J. Leukoc. Biol., 2012, 92, 1147-1154). This reactivation could enable new therapeutic methods for AIDS treatment (Zinchenko et al., J. Leukoc. Biol., 2012, 92, 1127-1129). A critical role of BRD4 in DNA replication of polyomaviruses has also been reported (Wang et al., PLoS Pathog., 2012, 8, doi:10.1371).

BET proteins are additionally involved in inflammation processes. BRD2-hypomorphic mice show reduced inflammation in adipose tissue (Wang et al., Biochem. J., 2009, 425:71-83). Infiltration of macrophages in white adipose tissue is also reduced in BRD2-deficient mice (Wang et al., Biochem. J., 2009, 425:71-83). It has also been shown that BRD4 regulates a number of genes involved in inflammation. In LPS-stimulated macrophages, a BRD4 inhibitor prevents the expression of inflammatory genes, for example IL-1 or IL-6 (Nicodeme et al., Nature, 2010, 468:1119-1123).

BET proteins are also involved in the regulation of the ApoA1 gene (Mirguet et al., Bioorg. Med. Chem. Lett., 2012, 22:2963-2967). The corresponding protein is part of high-density lipoprotein (HDL), which plays an important role in atherosclerosis (Smith, Arterioscler. Thromb. Vasc. Biol., 2010, 30:151-155). Through the stimulation of ApoA1 expression, BET protein inhibitors can increase the concentrations of cholesterol HDL and hence may potentially be useful for the treatment of atherosclerosis (Mirguet et al., Bioorg. Med. Chem. Lett., 2012, 22:2963-2967). The BET protein BRDT plays an essential role in spermatogenesis through the regulation of the expression of several genes important during and after meiosis (Shang et al., Development, 2007, 134:3507-3515; Matzuk et al., Cell, 2012, 150:673-684). In addition, BRDT is involved in the post-meiotic organization of chromatin (Dhar et al., J. Biol. Chem., 2012, 287:6387-6405). In vivo experiments in mice show that treatment with a BET inhibitor which also inhibits BRDT leads to a decrease in sperm production and infertility (Matzuk et al., Cell, 2012, 150:673-684).

All these studies show that the BET proteins play an essential role in various pathologies, and also in male fertility. It would therefore be desirable to find potent and selective inhibitors which prevent the interaction between the BET proteins and acetylated proteins. These novel inhibitors should also have suitable pharmacokinetic properties which allow inhibition of these interactions in vivo, i.e. in patients.

Tumour cells are furthermore distinguished by an uninhibited cell cycle process. The is due, firstly, to the loss of control proteins such as RB, p16, p21, p53 etc., and also to the activation of so-called accelerators of the cell cycle process, the cyclin-dependent kinases (CDKs). In pharmacy, CDKs are a recognized anti-tumour target protein. In addition to CDKs, novel cell cycle-regulating serine/threonine kinases, the Polo-like kinases, have been described which are involved not only in cell cylce regulation but also coordination with other processes during mitosis and cytokinesis (formation of the spindle apparatus, chromosome separation). Accordingly, this class of proteins represents an interesting point of attack for therapeutic intervention in proliferative diseases such as cancer (Descombes and Nigg. Embo J, 17; 1328ff, 1998; Glover et al. Genes Dev 12, 3777ff, 1998).

A high expression rate of Plk-1 has been found in non-small cell lung cancer (Wolf et al. Oncogene, 14, 543ff, 1997), in melanomas (Strebhardt et al. JAMA, 283, 479ff, 2000), in squamous cell carcinomas (Knecht et al. Cancer Res, 59, 2794ff, 1999) and in oesophageal carcinomas (Tokumitsu et al. Int J Oncol 15, 687ff, 1999).

A correlation of a high expression rate in tumour patients having a poor disgnosis has been demonstrated for various tumours (Strebhardt et al. JAMA, 283, 479ff, 2000, Knecht et al. Cancer Res, 59, 2794ff, 1999 and Tokumitsu et al. Int J Oncol 15, 687ff, 1999).

Constitutive expression of Plk-1 in NIH-3T3 cells results in malignant transformation (increased proliferation, growth in soft agar, colony formation and tumour development in nude mice (Smith et al. Biochem Biophys Res Comm, 234, 397ff., 1997).

Microinjections of Plk-1 antibodies in HeLa cells lead to defective mitosis (Lane et al.; Journal Cell Biol, 135, 1701ff, 1996).

Using a 20-mer antisense oligo, it was possible to inhibit the expression of Plk-1 in A549 cells and stop their viability. Also demonstrated was a marked anti-tumour action in nude mice (Mundt et al., Biochem Biophys Res Comm, 269, 377ff., 2000).

Microinjection of anti-Plk-1 antibodies into non-immortalized human Hs68 cells resulted, compared to HeLa cells, in a higher fraction of cells which remained at G2 during growth arrest and showed fewer signs of defective mitosis (Lane et al.; Journal Cell Biol, 135, 1701ff, 1996).

In contrast to the growth of tumour cells, the growth and the viability of primary human mesangial cells were not inhibited by antisense oligo molecules (Mundt et al., Biochem Biophys Res Comm, 269, 377ff., 2000).

Hitherto, in mammals, in addition of Plk-1, three further Polo kinases have been described which are induced as a mitogenic response and which exert their function in the G1 phase of the cell cycle. These are, firstly, the Prk/Plk-3 (the human homologue of the mouse-Fnk=fibroblast growth factor induced kinase; Wiest et al., Genes, Chromosomes & Cancer, 32: 384ff, 2001), Snk/Plk-2 (Serum induced kinase, Liby et al., DNA Sequence, 11, 527-33, 2001) and Sak/Plk4 (Fode et al., Proc. Natl. Acad. Sci. USA, 91, 6388ff; 1994).

The sequence identity within the Plk domains of the Polo family is between 40 and 60%, so that there are some interactions between inhibitors of one kinase with one or more other kinases of this family.

There is still a great need for active compounds for prophylaxis and treatment of disorders, especially of hyperproliferative disorders, and very particularly of neoplastic disorders.

It would therefore be desirable to have suitable compounds having dual inhibitory action and inhibiting both BET proteins and Plk proteins.

Surprisingly, it has now been found that substituted pyridopyrazinones have the desired properties, i.e. show BET-inhibitory, in particular BRD4-inhibitory, and simultaneously Plk-inhibitory, in particular Plk-1-inhibitory, action.

The compounds according to the invention are thus valuable active compounds for prophylactic and therapeutic use in the case of hyperproliferative disorders, especially in the case of neoplastic disorders. In addition, the compounds according to the invention can be used in the case of viral infections, in the case of neurodegenerative disorders, in the case of inflammation diseases, in the case of atherosclerotic disorders and in male fertility control.

The compounds according to the invention inhibit both the BET proteins and the Polo-like kinases, which is also the basis for their action for example against cancer, such as solid tumours and leukaemia, autoimmune disorders such as psoriasis, alopecia and multiple sclerosis, chemotherapeutics-induced alopecia and mucositis, cardiovascular disorders such as stenoses, arterioscleroses and restenoses, infectious disorders such as those caused, for example, by unicellular parasites such as trypanosoma, toxoplasma or plasmodium, or by fungi, nephrological disorders such as, for example, glomerulonephritis, chronic neurodegenerative disorders such as Huntington's disease, amyotrophic lateral sclerosis, Parkinson's disease, AIDS dementia and Alzheimer's disease, acute neurodegenerative disorders such as ischaemias of the brain and neurotraumata, viral infections such as, for example, cytomegalus infections, herpes, hepatitis B and C, and HIV disorders.

### PRIOR ART

The nomenclature applied in the assessment of the prior art (derived from the nomenclature software ACD Name batch, Version 12.01, from Advanced Chemical Development, Inc.) is illustrated by the following diagrams:

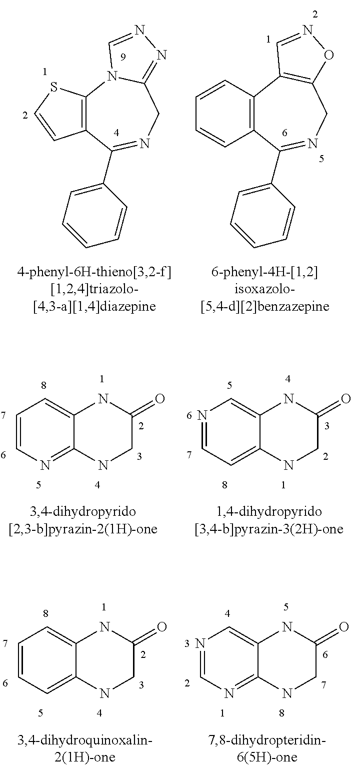

Based on the chemical structure, only very few types of BRD4 inhibitors have been described to date (Chun-Wa Chung et al., Progress in Medicinal Chemistry 2012, 51, 1-55).

The first published BRD4 inhibitors were diazepines. For example, phenylthienotriazolo-1,4-diazepines (4-phenyl-6H-thieno[3,2-f][1,2,4]triazolo[4,3-a][1,4]diazepines) are described in WO2009/084693 (Mitsubishi Tanabe Pharma Corporation) and as compound JQ1 in WO2011/143669 (Dana Farber Cancer Institute).

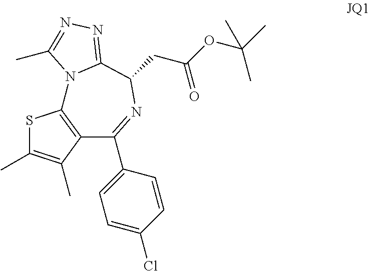 [SMILES: Cc1sc2c(c1C)C(c1ccc(Cl)cc1)=N[C@@H](CC(=O)OC(C)(C)C)c1nnc(C)n1-2]

Replacement of the thieno moiety by a benzo moiety also leads to active inhibitors (J. Med. Chem. 2011, 54, 3827-3838; E. Nicodeme et al., Nature 2010, 468, 1119). Further 4-phenyl-6H-thieno[3,2-f][1,2,4]triazolo[4,3-a][1,4]diazepines and related compounds having alternative rings as a fusion partner rather than the benzo unit are claimed generically or described explicitly in WO2012/075456 (Constellation Pharmaceuticals).

Azepines as BRD4 inhibitors are described in WO2012/075383 (Constellation Pharmaceuticals). This application relates to 6-substituted 4H-isoxazolo[5,4-d][2]benzazepines and 4H-isoxazolo[3,4-d][2]benzazepines, including those compounds which have optionally substituted phenyl at position 6, and also to analogues with alternative heterocyclic fusion partners rather than the benzo moiety, for example thieno- or pyridoazepines. Another structural class of BRD4 inhibitors described is that of 7-isoxazoloquinolines and related quinolone derivatives (Bioorganic & Medicinal Chemistry Letters 22 (2012) 2963-2967). WO2011/054845 (GlaxoSmithKline) describes further benzodiazepines as BRD4 inhibitors.

Some publications disclose compounds of a similar structure, some of which are described as inhibitors of cell cycle kinases, for example of Plk-1, but some of which are also directed to entirely different mechanisms of action and in some cases also to different indications. Dihydropyridopyrazinones and related bicyclic systems have been described in a series of patent applications.

WO 2006/005510 or US 2006/009457 (Boehringer Ingelheim) describes 1,4-dihydropyrido[3,4-b]pyrazin-3(2H)-one derivatives as inhibitors of Plk-1 for treatment of hyperproliferative disorders. The substances claimed are characterized by an anilinic group which is bonded via —NH— to C-7 of the dihydropyridopyrazinone skeleton and which is itself substituted in the para position by a carboxamide. In contrast, the compounds of the present invention have a substituted aminopyridine at the location of the anilinic group mentioned above.

WO 2013/071217 (OSI Pharmaceuticals) discloses mainly 7,8-dihydropteridin-6(5H)-ones, but also 1,4-dihydropyrido[3,4-b]pyrazin-3(2H)-one derivatives as inhibitors of kinases, in particular of RSK-1 and RSK-2, as medicaments, inter alia for the treatment of various neoplastic disorders. However, the compounds disclosed therein differ from the compounds according to the invention inter alia in the obligatory aromatic substitution at the nitrogen atom directly adjacent to the oxo group (N-5 in the dihydropteridones, or N-4 in the dihydropyrido[3,4-b]pyrazinones).

WO 2010/085570 (Takeda Pharmaceutical Company) describes inhibitors of poly-ADP-ribose polymerase (PARP) which are derived from a series of bi- and tricyclic skeletons, and which include 3,4-dihydropyrido[2,3-b]pyrazin-2(1H)-one derivatives, as medicaments for treatment of various diseases. The exemplary compounds disclosed therein differ from the compounds according to the invention in the position of the nitrogen in the pyridine moiety of the pyridopyridazine skeleton, and in the nature and position of the substitution present therein.

WO 2011/031965 (Gilead Sciences) describes 3-deazapteridinone derivatives (corresponds to 1,4-dihydropyrido[3,4-b]pyrazin-3(2H)-one derivatives) as modulators of Toll-like receptors for the treatment of various diseases. The substances disclosed therein differ from the compounds according to the invention inter alia in the obligatory amino substitution at C-5 and in the missing substitution at N-4.

WO 2003/020722 and WO 2004/076454 (Boehringer Ingelheim) disclose 7,8-dihydropteridin-6(5H)-ones as inhibitors of specific cell cycle kinases for treatment of hyperproliferative disorders.

WO 2006/018182 (Boehringer Ingelheim) describes pharmaceutical preparations of 7,8-dihydropteridin-6(5H)-ones in combination inter alia with various cytostatics for treatment of neoplastic disorders.

WO 2006/018185 (Boehringer Ingelheim) describes the use of 7,8-dihydropteridin-6(5H)-ones for treatment of various neoplastic disorders.

WO 2011/101369 (Boehringer Ingelheim), WO 2011/113293 (Jiangsu Hengrui Medicine), WO 2009/141575 (Chroma Therapeutics), WO 2009/071480 (Nerviano Medical Sciences) and also WO 2006/021378, WO 2006/021379 and WO 2006/021548 (likewise Boehringer Ingelheim) disclose further 7,8-dihydropteridin-6(5H)-one derivatives as inhibitors of Plk-1 for treating hyperproliferative disorders.

WO 2012/085176 (Hoffmann-La Roche AG) discloses tricyclic pyrazinone derivatives as inhibitors of janus kinases (JNK) for the treatment of various diseases.

WO 2008/117061 (Sterix Ltd) describes a series of bicyclic chemotypes, including 3,4-dihydroquinoxalin-2(1H)-one derivatives, as inhibitors of steroid sulphatase, for uses including inhibition of the growth of tumours.

WO 2006/050054, WO 2007/134169 and US 2009/0264384 (Nuada LLC) describe a series of bicyclic chemotypes, including 3,4-dihydroquinoxalin-2(1H)-one derivatives, as inhibitors of tumour necrosis factor alpha (TNF-α) and various isoforms of phosphodiesterase for treatment of inflammation disorders among others.

US 2006/0019961 (P. E. Mahaney et al.) describes substituted 3,4-dihydroquinoxalin-2(1H)-one derivatives as modulators of the oestrogen receptor for treatment of various inflammation disorders, cardiovascular disorders and autoimmune disorders.

The compounds according to the invention, in contrast, are substituted 1,4-dihydropyrido[3,4-b]pyrazin-3(2H)-one derivatives which differ structurally in various ways from the above-discussed chemotypes of BRD4 and Plk-1 inhibitors. Owing to the substantial structural differences, but also with a view to the structures themselves, it was not anticipated that the compounds claimed herein would have dual activity, i.e. that they would act in both a BRD4-inhibitory and Plk-inhibitory fashion. It is therefore surprising that the compounds according to the invention have a dual mode of action and therefore good inhibitory action in spite of the considerable structural differences.

It has now been found that compounds of the general formula (I)

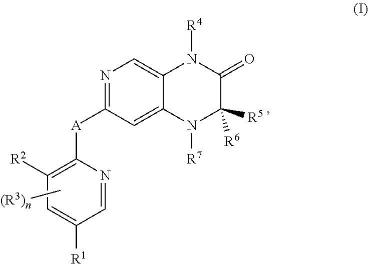

in which
A represents —NH— or —O—,
R¹ represents a —C(═O)NR⁸R⁹ or —S(═O)₂NR⁸R⁹ group, or
   represents oxazolin-2-yl which may optionally be mono- or disubstituted by C₁-C₃-alkyl-, or
   represents 5-membered monocyclic heteroaryl- which may optionally be mono-, di- or trisubstituted by identical or different substituents from the group consisting of halogen, cyano, C₁-C₄-alkyl-, C₂-C₄-alkenyl-, C₂-C₄-alkynyl-, halo-C₁-C₄-alkyl-, C₁-C₄-alkoxy-, halo-C₁-C₄-alkoxy-, C₁-C₄-alkylthio-, halo-C₁-C₄-alkylthio-, —NR¹⁰R¹¹, —C(═O)OR¹², —C(═O)N¹⁰R¹¹, —C(═O)₂R¹², —C(═O)₂R¹², —S(═O)₂NR¹⁰R¹¹,
R² represents hydrogen, halogen, cyano, C₁-C₄-alkyl-, C₂-C₄-alkenyl-, C₂-C₄-alkynyl-, halo-C₁-C₄-alkyl-, C₁-C₄-alkoxy-, halo-C₁-C₄-alkoxy-, C₁-C₄-alkylthio- or halo-C₁-C₄-alkylthio-,
R³ represents halogen, C₁-C₃-alkyl-, C₁-C₃-alkoxy- or cyano,
R⁴ represents methyl- or ethyl-,
R⁵ represents hydrogen or C₁-C₃-alkyl-,
R⁶ represents hydrogen or C₁-C₃-alkyl, or
R⁵ and R⁶ together with the carbon atom to which they are attached represent C₃-C₆-cycloalkyl,
R⁷ represents C₁-C₆-alkyl- which may optionally be monosubstituted by phenyl-, C₃-C₈-cycloalkyl-, or 4- to 8-membered heterocycloalkyl-,
   in which phenyl- for its part may optionally be mono-, di- or trisubstituted by identical or different substituents from the group consisting of: halogen, cyano, C₁-C₄-alkyl-, C₂-C₄-alkenyl-, C₂-C₄-alkynyl-, C₁-C₄-alkoxy-, halo-C₁-C₄-alkyl-, halo-C₁-C₄-alkoxy-, and
   in which C₃-C₈-cycloalkyl- and 4- to 8-membered heterocycloalkyl- for their part may optionally be mono- or disubstituted by C₁-C₃-alkyl-, or
   represents C₃-C₈-cycloalkyl- or 4- to 8-membered heterocycloalkyl- which may optionally be mono- or disubstituted by C₁-C₃-alkyl-,
R⁸ represents C₁-C₆-alkyl- which may optionally be mono-, di- or trisubstituted by identical or different substituents from the group consisting of: hydroxy, oxo, fluorine, cyano, C₁-C₄-alkoxy-, halo-C₁-C₄-alkoxy-, —NR¹⁰R¹¹, C₃-C₈-cycloalkyl-, C₄-C₈-cycloalkenyl-, 4- to 8-membered heterocycloalkyl-, 4-bis 8-membered heterocycloalkenyl-, C₅-C₁₁-spirocycloalkyl-, C₅-C₁₁-heterospirocycloalkyl-, bridged C₆-C₁₂-cycloalkyl-, bridged C₆-C₁₂-heterocycloalkyl-, C₆-C₁₂-bicycloalkyl-, C₆-C₁₂-heterobicycloalkyl-, phenyl- and 5- to 6-membered heteroaryl-, in which C₃-C₈-cycloalkyl-, C₄-C₈-cycloalkenyl-, 4- to 8-membered heterocycloalkyl-, 4- to 8-membered heterocycloalkenyl-, C₅-C₁₁-spirocycloalkyl-, C₅-C₁₁-heterospirocycloalkyl-, bridged C₆-C₁₂-cycloalkyl-, bridged C₆-C₁₂-heterocycloalkyl-, C₆-C₁₂-bicycloalkyl-, C₆-C₁₂-heterobicycloalkyl- may optionally be mono- or disubstituted by identical or different substituents from the group consisting of: hydroxy, fluorine, oxo, cyano, C₁-C₃-alkyl-, fluoro-C₁-C₃-alkyl-, C₃-C₆-cycloalkyl-, cyclopropylmethyl-, C₁-C₃-alkylcarbonyl-, C₁-C₄-alkoxycarbonyl- and —NR¹⁰R¹¹, and
   in which phenyl and 5- to 6-membered heteroaryl may optionally be mono- or disubstituted by identical or different substituents from the group consisting of: halogen, cyano, trifluoromethyl-, C₁-C₃-alkyl-, C₁-C₃-alkoxy-,
   or represents C₃-C₆-alkenyl or C₃-C₆-alkynyl,
   or represents fluoro-C₁-C₃-alkyl- which may optionally be monosubstituted by cyano or hydroxy,
   or represents C₃-C₈-cycloalkyl-, C₄-C₈-cycloalkenyl-, C₅-C₁₁-spirocycloalkyl-, bridged C₆-C₁₂-cycloalkyl- or C₆-C₁₂-bicycloalkyl- which may optionally be mono- or disubstituted by identical or different substituents from the group consisting of: hydroxy, oxo, cyano, fluorine, C₁-C₃-alkyl, C₁-C₃-alkoxy, trifluoromethyl, —NR¹⁰R¹¹,
   or represents 4- to 8-membered heterocycloalkyl-, 4- to 8-membered heterocycloalkenyl-, C₅-C₁₁-heterospirocycloalkyl-, bridged C₆-C₁₂-heterocycloalkyl- or C₆-C₁₂-heterobicycloalkyl- which may optionally be mono- or disubstituted by identical or different substituents from the group consisting of: hydroxy, fluorine, oxo, cyano, C₁-C₃-alkyl-, fluoro-C₁-C₃-alkyl-, C₃-C₆-cycloalkyl-, cyclopropylmethyl-, C₁-C₃-alkylcarbonyl-, C₁-C₄-alkoxycarbonyl- and —NR¹⁰R¹¹,
R⁹ represents hydrogen or represents C₁-C₃-alkyl- which is optionally mono- or disubstituted by identical or different substituents from the group consisting of hydroxy, oxo, C₁-C₃-alkoxy-, or represents fluoro-C₁-C₃-alkyl, or
R⁸ and R⁹ together with the nitrogen atom to which they are attached represent 4- to 8-membered heterocycloalkyl, 4- to 8-membered heterocycloalkenyl-, C₅-C₁₁-heterospirocycloalkyl-, bridged C₆-C₁₂-heterocycloalkyl- or C₆-C₁₂-heterobicycloalkyl- which may optionally be mono- or disubstituted by identical or different substituents from the group consisting of: hydroxy, fluorine, oxo, cyano, C₁-C₃-alkyl-, fluoro-C₁-C₃-alkyl-, C₃-C₆-cycloalkyl-, cyclopropylmethyl-, C₁-C₃-alkylcarbonyl-, C₁-C₄-alkoxycarbonyl- and —NR¹⁰R¹¹,
R¹⁰ and R¹¹ independently of one another represent hydrogen or represent C₁-C₃-alkyl which is optionally mono- or disubstituted by identical or different substituents from the group consisting of hydroxy, oxo, C₁-C₃-alkoxy-, or represents fluoro-C₁-C₃-alkyl, or
R¹⁰ and R¹¹ together with the nitrogen atom to which they are attached represent 4- to 8-membered heterocycloalkyl- which may optionally be mono- or disubstituted by identical or different substituents from the group consisting of: hydroxy, fluorine, oxo, cyano, C₁-C₃-alkyl-, fluoro-C₁-C₃-alkyl-, C₃-C₆-cycloalkyl-, cyclopropylmethyl-, C₁-C₃-alkylcarbonyl- and C₁-C₄-alkoxycarbonyl-,
R¹² represents C₁-C₆-alkyl- or phenyl-C₁-C₃-alkyl-, and
n represents 0 or 1,
   and their diastereomers, racemates, metabolites, polymorphs and physiologically acceptable salts surprisingly inhibit the interaction between BET proteins, in particular BRD4, and an acetylated histone 4 peptide and the kinase Plk-1 and therefore, owing to the dual mechanism mentioned, have the properties described above and in particular inhibit the growth of cancer cells.

Preference is given to those compounds of the general formula (I)

in which
A represents —NH—,
R¹ represents a —C(═O)NR⁸R⁹ or —S(═O)₂NR⁸R⁹ group, or
   represents oxazolin-2-yl which may optionally be mono- or disubstituted by C₁-C₃-alkyl-, or
   represents oxazolyl-, thiazolyl-, oxadiazolyl- or thiadiazolyl- which may optionally be mono- or disubstituted by identical or different substituents from the group consisting of halogen, cyano, C₁-C₃-alkyl-, trifluoromethyl-, C₁-C₃-alkoxy-, trifluoromethoxy- and —NR¹⁰R¹¹,
R² represents hydrogen, fluorine, chlorine, cyano, methyl-, ethyl-, methoxy- or ethoxy-,
R³ represents fluorine, chlorine or methyl-,
R⁴ represents methyl-,
R⁵ represents hydrogen, methyl- or ethyl-,
R⁶ represents hydrogen, methyl- or ethyl-,
R⁷ represents C₃-C₅-alkyl-, or
   represents methyl- or ethyl- which may be monosubstituted by phenyl- or 4- to 8-membered heterocycloalkyl-,
   in which phenyl- for its part may optionally be mono- or disubstituted by identical or different substituents from the group consisting of: fluorine, chlorine, bromine, cyano, C₁-C₃-alkyl-, C₁-C₃-alkoxy-, trifluoromethyl-, and
   in which 4- to 8-membered heterocycloalkyl- for its part may optionally be mono- or disubstituted by methyl-, or
   represents C₃-C₆-cycloalkyl- or 4- to 8-membered heterocycloalkyl- which may optionally be mono- or disubstituted by methyl-,
R⁸ represents C₁-C₆-alkyl- which may optionally be mono-, di- or trisubstituted by identical or different substituents from the group consisting of: hydroxy, oxo, fluorine, cyano, C₁-C₃-alkoxy-, fluoro-C₁-C₃-alkoxy-, —NR¹⁰R¹¹, 4- to 8-membered heterocycloalkyl-, phenyl- and 5- to 6-membered heteroaryl-,
   in which the 4- to 8-membered heterocycloalkyl- may optionally be monosubstituted by: hydroxy, oxo, C₁-C₃-alkyl-, fluoro-C₁-C₃-alkyl-, cyclopropyl-, cyclopropylmethyl-, acetyl- or tert-butoxycarbonyl-,
   and in which phenyl and 5- to 6-membered heteroaryl may optionally be mono- or disubstituted by identical or different substituents from the group consisting of: fluorine, chlorine, cyano, trifluoromethyl-, methyl-, methoxy-, or represents fluoro-C₁-C₃-alkyl-,
   or represents C₃-C₆-cycloalkyl- which may optionally be mono- or disubstituted by identical or different substituents from the group consisting of: hydroxy, oxo, cyano, fluorine, —NR¹⁰R¹¹,
   or represents 4- to 8-membered heterocycloalkyl-, C₆-C₈-heterospirocycloalkyl-, bridged C₆-C₁₀-heterocycloalkyl- or C₆-C₁₀-heterobicycloalkyl- which may optionally be mono- or disubstituted by identical or different substituents from the group consisting of: hydroxy, oxo, C₁-C₃-alkyl-, fluoro-C₁-C₃-alkyl-, cyclopropyl-, cyclopropylmethyl-, acetyl- and tert-butoxycarbonyl-,
R⁹ represents hydrogen or C₁-C₃-alkyl, or
R⁸ and R⁹ together with the nitrogen atom to which they are attached represent 4- to 8-membered heterocycloalkyl-, C₆-C₈-heterospirocycloalkyl-, bridged C₆-C₁₀-heterocycloalkyl- or C₆-C₁₀-heterobicycloalkyl-,
   which may optionally be mono- or disubstituted by identical or different substituents from the group consisting of: hydroxy, oxo, C₁-C₃-alkyl-, fluoro-C₁-C₃-alkyl-, cyclopropyl-, cyclopropylmethyl-, acetyl- and tert-butoxycarbonyl-,
R¹⁰ and R¹¹ independently of one another represent hydrogen or represent C₁-C₃-alkyl which is optionally monosubstituted by hydroxy or oxo or represent trifluoromethyl-, or
R¹⁰ and R¹¹ together with the nitrogen atom to which they are attached represent 4- to 7-membered heterocycloalkyl- which may optionally be mono- or disubstituted by identical or different substituents from the group consisting of: hydroxy, oxo, C₁-C₃-alkyl-, fluoro-C₁-C₃-alkyl-, cyclopropyl-, cyclopropylmethyl-, acetyl- and tert-butoxycarbonyl-, and
n represents 0 or 1,
   and diastereomers, racemates, polymorphs and physiologically acceptable salts thereof.

Particular preference is given to those compounds of the general formula (I)

in which
A represents —NH—,
R¹ represents a —C(═O)NR⁸R⁹ or —S(═O)₂NR⁸R⁹ group, or
   represents oxazolin-2-yl which may optionally be mono- or disubstituted by C₁-C₃-alkyl-,
R² represents hydrogen, methyl-, ethyl- or methoxy-,
R⁴ represents methyl-,
R⁵ represents methyl- or ethyl-,
R⁶ represents hydrogen,
R⁷ represents C₃-C₅-alkyl-, or
   represents methyl-monosubstituted by phenyl- or 4- to 6-membered heterocycloalkyl-,
   in which phenyl- for its part may optionally be mono- or disubstituted by identical or different substituents from the group consisting of: fluorine, chlorine, cyano, methyl-, methoxy-, and
   in which 4- to 6-membered heterocycloalkyl- for its part may optionally be monosubstituted by methyl-, or
   represents C₃-C₆-cycloalkyl- or represents 4- to 6-membered heterocycloalkyl-,
R⁸ represents C₁-C₄-alkyl- which may optionally be mono- or disubstituted by hydroxy, C₁-C₃-alkoxy-, —NR¹⁰R¹¹, 4- to 8-membered heterocycloalkyl, phenyl or 5- to 6-membered heteroaryl,
   in which the 4- to 8-membered heterocycloalkyl- may optionally be monosubstituted by: oxo, C₁-C₃-alkyl, fluoro-C₁-C₃-alkyl-, cyclopropyl- or cyclopropylmethyl-,
   and in which phenyl and 5- to 6-membered heteroaryl may optionally be mono- or disubstituted by identical or different substituents from the group consisting of: fluorine, chlorine, cyano, trifluoromethyl-, methyl- and methoxy-,
   or represents fluoro-C₁-C₃-alkyl-,
   or represents C₃-C₆-cycloalkyl- which may optionally be monosubstituted by
   hydroxy, fluorine or —NR¹⁰R¹¹,
   or represents 4- to 8-membered heterocycloalkyl- which may optionally be mono- or disubstituted by identical or different substituents from the group consisting of: oxo, C₁-C₃-alkyl, fluoro-C₁-C₃-alkyl-, cyclopropyl- and cyclopropylmethyl-,
R⁹ represents hydrogen or methyl-, or
R⁸ and R⁹ together with the nitrogen atom to which they are attached represent 5- to 6-membered heterocycloalkyl- which may optionally be mono- or disubstituted by identical or different substituents from the group consisting of: oxo, C₁-C₃-alkyl, fluoro-C₁-C₃-alkyl-, cyclopropyl- and cyclopropylmethyl-,
R¹⁰ and R¹¹ independently of one another represent hydrogen or represent C₁-C₃-alkyl-, or
R¹⁰ and R¹¹ together with the nitrogen atom to which they are attached represent 4- to 7-membered heterocycloalkyl- which may optionally be mono- or disubstituted by identical or different substituents from the group consisting of: oxo, C₁-C₃-alkyl-, fluoro-C₁-C₃-alkyl-, cyclopropyl- or cyclopropylmethyl-, and
n represents 0,
   and diastereomers, racemates, polymorphs and physiologically acceptable salts thereof.

Very particular preference is given to those compounds of the general formula (I)

in which
A represents —NH—,
R¹ represents a —C(═O)NR⁸R⁹ group, or
   represents oxazolin-2-yl which may optionally be mono- or disubstituted by C₁-C₃-alkyl-,
R² represents methyl-, ethyl- or methoxy-,
R⁴ represents methyl-,
R⁵ represents methyl- or ethyl-,
R⁶ represents hydrogen,
R⁷ represents C₃-C₅-alkyl-, or
   represents C₃-C₆-cycloalkyl,
R⁸ represents C₁-C₃-alkyl- which may optionally be monosubstituted by hydroxy, C₁-C₃-alkoxy-, phenyl- or 5- to 6-membered heteroaryl-,
   in which phenyl and 5- to 6-membered heteroaryl may optionally be mono- or disubstituted by identical or different substituents from the group consisting of: fluorine, chlorine, methyl- and methoxy-,
   or represents fluoro-C₁-C₃-alkyl-,
   or represents C₃-C₆-cycloalkyl,
   or represents 4- to 8-membered heterocycloalkyl- which may optionally be mono- or disubstituted by identical or different substituents from the group consisting of: oxo and C₁-C₃-alkyl-,
R⁹ represents hydrogen,
n represents 0,
   and diastereomers, racemates, polymorphs and physiologically acceptable salts thereof.

Exceptional preference is given to those compounds of the general formula (I)

in which
A represents —NH—,
R¹ represents a —C(═O)NR⁸R⁹ group, or
   represents oxazolin-2-yl which may optionally be mono- or disubstituted by methyl-,
R² represents methyl-, ethyl- or methoxy-,
R⁴ represents methyl-,
R⁵ represents methyl- or ethyl-,
R⁶ represents hydrogen,
R⁷ represents cyclopentyl-,
R⁸ represents C₁-C₄-alkyl- which may optionally by monosubstituted by hydroxy, methoxy- or pyridinyl-,
   or represents fluoro-C₁-C₂-alkyl-,
   or represents C₃-C₆-cycloalkyl,
   or represents 4- to 8-membered heterocycloalkyl- which may optionally be mono- or disubstituted by identical or different substituents from the group consisting of: oxo and C₁-C₃-alkyl-,
R⁹ represents hydrogen,
n represents 0,
   and diastereomers, racemates, polymorphs and physiologically acceptable salts thereof.

Even more preference is given to those compounds of the general formula (I) in which
A represents —NH—,
R¹ represents a —C(═O)NR⁸R⁹ group, or
   represents oxazolin-2-yl- which is disubstituted by methyl-,
R² represents methyl-, ethyl- or methoxy-,
R⁴ represents methyl-,
R⁵ represents methyl- or ethyl-,
R⁶ represents hydrogen,
R⁷ represents cyclopentyl-,
R⁸ represents C₁-C₄-alkyl- which may optionally by monosubstituted by hydroxy, methoxy- or pyridinyl-,
   or represents 2,2,2-trifluoroethyl-,
   or represents cyclopropyl- or cyclohexyl-,
   or represents piperidinyl, azepanyl or tetrahydropyranyl which may optionally be mono- or disubstituted by identical or different substituents from the group consisting of: oxo and methyl,
R⁹ represents hydrogen,
n represents 0,
   and diastereomers, racemates, polymorphs and physiologically acceptable salts thereof.

Preference is given to compounds of the general formula (I) in which A represents —NH—.

Preference is given to compounds of the general formula (I) in which A represents —O—.

Preference is given to compounds of the general formula (I) in which R¹ represents —C(═O)NR⁸R⁹.

Preference is given to compounds of the general formula (I) in which R¹ represents —S(═O)₂NR⁸R⁹.

Preference is given to compounds of the general formula (I) in which R¹ represents oxazolin-2-yl-which may optionally be mono- or disubstituted identically or differently by C₁-C₃-alkyl-.

In the general formula (I), R¹ may represent 5-membered monocyclic heteroaryl- which may optionally be mono-, di- or trisubstituted by identical or different substituents from the group consisting of halogen, cyano, C₁-C₄-alkyl-, C₂-C₄-alkenyl-, C₂-C₄-alkynyl-, halo-C₁-C₄-alkyl-, C₁-C₄-alkoxy-, halo-C₁-C₄-alkoxy-, C₁-C₄-alkylthio-, halo-C₁-C₄-alkylthio-, —NR¹⁰R¹¹, —C(═O)OR¹², —C(═O)N¹⁰R¹¹, —C(═O)R¹², —S(═O)₂R¹², —S(═O)₂NR¹⁰R¹¹.

Preference is given to compounds of the general formula (I) in which R¹ represents oxazolyl-, thiazolyl-, oxadiazolyl- or thiadiazolyl- which may optionally be mono- or disubstituted by identical or different substituents from the group consisting of halogen, cyano, C₁-C₃-alkyl-, trifluoromethyl-, C₁-C₃-alkoxy-, trifluoromethoxy- and —NR¹⁰R¹¹.

Preference is given to compounds of the general formula (I) in which R² represents hydrogen, fluorine, chlorine, cyano, methyl-, ethyl-, methoxy- or ethoxy-.

Preference is given to compounds of the general formula (I) in which R² represents hydrogen, methyl-, ethyl- or methoxy-.

Preference is given to compounds of the general formula (I) in which R² represents hydrogen.

Particular preference is given to compounds of the general formula (I) in which R² represents methyl-, ethyl- or methoxy-.

Particular preference is given to compounds of the general formula (I) in which R² represents methyl-.

Particular preference is given to compounds of the general formula (I) in which R² represents ethyl-.

Particular preference is given to compounds of the general formula (I) in which R² represents methoxy-.

Preference is given to compounds of the general formula (I) in which R³ represents fluorine, chlorine or methyl-.

Preference is given to compounds of the general formula (I) in which R⁴ represents hydrogen, methyl- or ethyl-.

Preference is given to compounds of the general formula (I) in which R⁴ represents methyl- or ethyl-.

Preference is given to compounds of the general formula (I) in which R⁴ represents ethyl-.

Particular preference is given to compounds of the general formula (I) in which R⁴ represents methyl-.

Preference is given to compounds of the general formula (I) in which R⁵ represents hydrogen, methyl- or ethyl-.

Preference is given to compounds of the general formula (I) in which R⁵ represents methyl- or ethyl-.

Preference is given to compounds of the general formula (I) in which R⁵ represents ethyl-.

Preference is given to compounds of the general formula (I) in which R⁵ represents methyl-.

Preference is given to compounds of the general formula (I) in which one substituent in each case from R⁵ and R⁶ represents methyl- and one represents hydrogen, so as to result in a racemate with respect to the stereocentre formed from R⁵, R⁶ and the carbon atom bonded to R⁵ and R⁶.

Preference is given to compounds of the general formula (I) in which one substituent in each case from R⁵ and R⁶ represents methyl- and one represents hydrogen, so as to result in an isomer mixture in which the (R) form predominates with respect to the stereocentre formed from R⁵, R⁶ and the carbon atom bonded to R⁵ and R⁶.

Particular preference is given to compounds of the general formula (I) in which R⁵ represents methyl- and R⁶ represents hydrogen.

Particular preference is given to compounds of the general formula (I) in which R⁵ represents ethyl- and R⁶ represents hydrogen.

Preference is given to compounds of the general formula (I) in which R⁷ represents unsubstituted C₃-C₅-alkyl- or

represents methyl- which is monosubstituted by phenyl- or 4- to 6-membered heterocycloalkyl-, in which phenyl- for its part may optionally be mono- or disubstituted by identical or different substituents from the group consisting of: fluorine, chlorine, cyano, methyl-, methoxy-, and

in which 4- to 6-membered heterocycloalkyl- for its part may optionally be monosubstituted by methyl-, or

represents C₃-C₆-cycloalkyl-, or represents 4- to 6-membered heterocycloalkyl-.

Preference is given to compounds of the general formula (I) in which R⁷ represents unsubstituted C₃-C₅-alkyl.

Preference is given to compounds of the general formula (I) in which R⁷ represents C₃-C₅-alkyl.

Preference is given to compounds of the general formula (I) in which R⁷ represents methyl- which is monosubstituted by phenyl-,

in which phenyl- for its part may optionally be mono- or disubstituted by identical or different substituents from the group consisting of: fluorine, chlorine, cyano, methyl-, methoxy-.

Preference is given to compounds of the general formula (I) in which R⁷ represents methyl- which is monosubstituted by 4- to 6-membered heterocycloalkyl-,

in which 4- to 6-membered heterocycloalkyl- for its part may optionally be monosubstituted by methyl-.

Preference is given to compounds of the general formula (I) in which R⁷ represents C₃-C₆-cycloalkyl-.

Preference is given to compounds of the general formula (I) in which R⁷ represents 4- to 6-membered heterocycloalkyl-.

Particular preference is given to compounds of the general formula (I) in which R⁷ represents cyclopentyl-.

Preference is given to compounds of the general formula (I) in which R⁸ represents C₁-C₄-alkyl which may optionally be monosubstituted by hydroxy, C₁-C₃-alkoxy-, —NR¹⁰R¹¹, 4- to 8-membered heterocycloalkyl-, phenyl- or 5- to 6-membered heteroaryl-,

in which the 4- to 8-membered heterocycloalkyl- may optionally be monosubstituted by: oxo, C₁-C₃-alkyl, fluoro-C₁-C₃-alkyl-, cyclopropyl- or cyclopropylmethyl-,

and in which phenyl and 5- to 6-membered heteroaryl may optionally be mono- or disubstituted by identical or different substituents from the group consisting of: fluorine, chlorine, cyano, trifluoromethyl-, methyl- and methoxy-,

or represents fluoro-C₁-C₃-alkyl-,

or represents C₃-C₆-cycloalkyl- which may optionally be monosubstituted by hydroxy, fluorine or —NR¹⁰R¹¹,

or represents 4- to 8-membered heterocycloalkyl- which may optionally be mono- or disubstituted by identical or different substituents from the group consisting of: oxo, C₁-C₃-alkyl, fluoro-C₁-C₃-alkyl-, cyclopropyl- and cyclopropylmethyl-.

Preference is given to compounds of the general formula (I) in which R⁸ represents C₁-C₄-alkyl which may optionally be monosubstituted by hydroxy, C₁-C₃-alkoxy-, —NR¹⁰R¹¹, 4- to 8-membered heterocycloalkyl-, phenyl- or 5- to 6-membered heteroaryl-,

in which the 4- to 8-membered heterocycloalkyl- may optionally be monosubstituted by: oxo, C₁-C₃-alkyl, fluoro-C₁-C₃-alkyl-, cyclopropyl- or cyclopropylmethyl-,

and in which phenyl and 5- to 6-membered heteroaryl may optionally be mono- or disubstituted by identical or different substituents from the group consisting of: fluorine, chlorine, cyano, trifluoromethyl-, methyl- and methoxy-.

Preference is given to compounds of the general formula (I) in which R⁸ represents C₃-C₆-cycloalkyl- which may optionally be monosubstituted by hydroxy, fluorine or —NR¹⁰R¹¹.

Preference is given to compounds of the general formula (I) in which R⁸ represents 4- to 8-membered heterocycloalkyl- which may optionally be mono- or disubstituted by identical or different substituents from the group consisting of: oxo, C₁-C₃-alkyl, fluoro-C₁-C₃-alkyl-, cyclopropyl- and cyclopropylmethyl-.

Particular preference is given to compounds of the general formula (I) in which R⁸ represents C₁-C₃-alkyl- which may optionally be monosubstituted by —NR¹⁰R¹¹, hydroxy, C₁-C₃-alkoxy-, phenyl- or 5- to 6-membered heteroaryl-,

in which phenyl and 5- to 6-membered heteroaryl may optionally be mono- or disubstituted by identical or different substituents from the group consisting of: fluorine, chlorine, methyl- and methoxy-,

or represents fluoro-C₁-C₃-alkyl-,

or represents C₃-C₆-cycloalkyl,

or represents 4- to 6-membered heterocycloalkyl- which may optionally be monosubstituted by: oxo or methyl-.

Particular preference is given to compounds of the general formula (I) in which R⁸ represents C₁-C₃-alkyl- which may optionally be monosubstituted by —NR¹⁰R¹¹, hydroxy, C₁-C₃-alkoxy-, phenyl- or 5- to 6-membered heteroaryl-,

in which phenyl and 5- to 6-membered heteroaryl may optionally be mono- or disubstituted by identical or different substituents from the group consisting of: fluorine, chlorine, methyl- and methoxy-.

Particular preference is given to compounds of the general formula (I) in which R⁸ represents fluoro-C₁-C₃-alkyl.

Particular preference is given to compounds of the general formula (I) in which R⁸ represents C₃-C₆-cycloalkyl.

Particular preference is given to compounds of the general formula (I) in which R⁸ is 4- to 6-membered heterocycloalkyl- which may optionally be monosubstituted by: oxo and C₁-C₃-alkyl-.

Preference is given to compounds of the general formula (I) in which R⁹ represents hydrogen or methyl-.

Preference is given to compounds of the general formula (I) in which R⁹ represents methyl-.

Particular preference is given to compounds of the general formula (I) in which R⁹ represents hydrogen.

Preference is given to compounds of the general formula (I) in which R⁸ and R⁹ together with the nitrogen atom to which they are bonded are 4- to 8-membered heterocycloalkyl-, C₆-C₈-heterospirocycloalkyl-, bridged C₆-C₁₀-heterocycloalkyl- or C₆-C₁₀-heterobicycloalkyl-, which may optionally be mono- or disubstituted by identical or different substituents from the group consisting of: hydroxy, oxo, C₁-C₃-alkyl-, fluoro-C₁-C₃-alkyl-, cyclopropyl-, cyclopropylmethyl-, acetyl- or tert-butoxycarbonyl-.

Preference is given to compounds of the general formula (I) in which R⁸ and R⁹ together with the nitrogen atom to which they are bonded are 4- to 8-membered heterocycloalkyl-, which may optionally be mono- or disubstituted by identical or different substituents from the group consisting of: hydroxy, oxo, C₁-C₃-alkyl-, fluoro-C₁-C₃-alkyl-, cyclopropyl-, cyclopropylmethyl-, acetyl- or tert-butoxycarbonyl-.

Particular preference is given to compounds of the general formula (I) in which R⁸ and R⁹ together with the nitrogen atom to which they are bonded are 5- to 6-membered heterocycloalkyl- or C₆-C₈-heterospirocycloalkyl-, which may optionally be mono- or disubstituted by identical or different substituents from the group consisting of: oxo, C₁-C₃-alkyl, fluoro-C₁-C₃-alkyl-, cyclopropyl- or cyclopropylmethyl-.

Particular preference is given to compounds of the general formula (I) in which R⁸ and R⁹ together with the nitrogen atom to which they are attached represent 5- to 6-membered heterocycloalkyl-which may optionally be mono- or disubstituted by identical or different substituents from the group consisting of: oxo, C₁-C₃-alkyl, fluoro-C₁-C₃-alkyl-, cyclopropyl- and cyclopropylmethyl-.

Preference is given to compounds of the general formula (I) in which R¹⁰ and R¹¹ independently of one another represent hydrogen or represent C₁-C₃-alkyl- which is optionally monosubstituted by hydroxy or oxo or represent trifluoromethyl-.

Particular preference is given to compounds of the general formula (I) in which R¹⁰ and R¹¹ are each independently hydrogen or C₁-C₃-alkyl-.

Preference is given to compounds of the general formula (I) in which R¹⁰ and R¹¹ together with the nitrogen atom to which they are attached represent 4- to 7-membered heterocycloalkyl- which may optionally be mono- or disubstituted by identical or different substituents from the group consisting of: oxo, C₁-C₃-alkyl, fluoro-C₁-C₃-alkyl-, cyclopropyl- and cyclopropylmethyl-.

Preference is given to compounds of the general formula (I) in which n represents the number 1.

Preference is given to compounds of the general formula (I) in which n represents the number 0 or the number 1.

Particular preference is given to compounds of the general formula (I) in which n represents the number 0.

Particular preference is given to compounds of the general formula (I) in which n represents the number 0 and in which A represents —NH—, R⁴ represents methyl, R⁵ represents methyl- or ethyl- and R⁶ represents hydrogen.

The specific radical definitions given in the particular combinations or preferred combinations of radicals are, irrespective of the particular combinations of radicals specified, also replaced as desired by radical definitions of other combination.

Very particular preference is given to combinations of two or more of the abovementioned preferred ranges.

Very particular preference is given to the following compounds of the general formula (I):
6-{[1-cyclopentyl-2,4-dimethyl-3-oxo-1,2,3,4-tetrahydropyrido[3,4-b]pyrazin-7-yl]amino}-N-cyclopropyl-5-methoxypyridine-3-carboxamide,
6-{[(2R)-1-cyclopentyl-2,4-dimethyl-3-oxo-1,2,3,4-tetrahydropyrido[3,4-b]pyrazin-7-yl]amino}-N-cyclopropyl-5-methoxypyridine-3-carboxamide,
6-{[(2R)-1-cyclopentyl-2-ethyl-4-methyl-3-oxo-1,2,3,4-tetrahydropyrido[3,4-b]pyrazin-7-yl]amino}-N-cyclopropyl-5-ethylpyridine-3-carboxamide,
6-[(1-cyclopentyl-2-ethyl-1,2,3,4-tetrahydro-4-methyl-3-oxopyrido[3,4-b]pyrazin-7-yl)amino]-N-cyclopropyl-5-methoxy-3-pyridinecarboxamide,
6-[[(2R)-1-cyclopentyl-2-ethyl-1,2,3,4-tetrahydro-4-methyl-3-oxopyrido[3,4-b]pyrazin-7-yl)amino]-N-cyclopropyl-5-methoxy-3-pyridinecarboxamide,
6-[(1-cyclopentyl-2,4-dimethyl-3-oxo-1,2,3,4-tetrahydropyrido[3,4-b]pyrazin-7-yl)amino]-5-methoxy-N-(1-methylpiperidin-4-yl)pyridine-3-carboxamide,
6-[[(2R)-1-cyclopentyl-2-ethyl-1,2,3,4-tetrahydro-4-methyl-3-oxopyrido[3,4-b]pyrazin-7-yl]amino]-5-methoxy-N-(2,2,2-trifluoroethyl)-3-pyridinecarboxamide,
6-[[(2R)-1-cyclopentyl-2-ethyl-1,2,3,4-tetrahydro-4-methyl-3-oxopyrido[3,4-b]pyrazin-7-yl]amino]-5-methoxy-N-(2-methoxyethyl)-3-pyridinecarboxamide,
6-{[(2R)-1-cyclopentyl-2-ethyl-4-methyl-3-oxo-1,2,3,4-tetrahydropyrido[3,4-b]pyrazin-7-yl]amino}-5-ethyl-N-[(3R)-2-oxoazepan-3-yl]pyridine-3-carboxamide,
6-[[(2R)-1-cyclopentyl-2-ethyl-1,2,3,4-tetrahydro-4-methyl-3-oxopyrido[3,4-b]pyrazin-7-yl]amino]-5-methoxy-N-(tetrahydro-2H-pyran-4-yl)-3-pyridinecarboxamide,
6-[[(2R)-1-cyclopentyl-2-ethyl-1,2,3,4-tetrahydro-4-methyl-3-oxopyrido[3,4-b]pyrazin-7-yl]amino]-N-(2-hydroxy-1,1-dimethylethyl)-5-methoxy-3-pyridinecarboxamide,
6-[[(2R)-1-cyclopentyl-2-ethyl-1,2,3,4-tetrahydro-4-methyl-3-oxopyrido[3,4-b]pyrazin-7-yl]amino]-5-methoxy-N-(3-pyridinylmethyl)-3-pyridinecarboxamide,
6-{[(2R)-1-cyclopentyl-2-ethyl-4-methyl-3-oxo-1,2,3,4-tetrahydropyrido[3,4-b]pyrazin-7-yl]amino}-N-cyclopropyl-5-methylpyridine-3-carboxamide,
6-[[(2R)-1-cyclopentyl-2-ethyl-1,2,3,4-tetrahydro-4-methyl-3-oxopyrido[3,4-b]pyrazin-7-yl]amino]-5-methoxy-N-(1-methyl-4-piperidinyl)-3-pyridinecarboxamide,
6-{[(2R)-1-cyclopentyl-2-ethyl-4-methyl-3-oxo-1,2,3,4-tetrahydropyrido[3,4-b]pyrazin-7-yl]amino}-5-methylpyridine-3-carboxamide,
6-[[(2R)-1-cyclopentyl-2-ethyl-1,2,3,4-tetrahydro-4-methyl-3-oxopyrido[3,4-b]pyrazin-7-yl]amino]-N-[(3R)-hexahydro-2-oxo-1H-azepin-3-yl]-5-methoxy-3-pyridinecarboxamide,
6-{[(2R)-1-cyclopentyl-2-ethyl-4-methyl-3-oxo-1,2,3,4-tetrahydropyrido[3,4-b]pyrazin-7-yl]amino}-5-methyl-N-(tetrahydro-2H-pyran-4-yl)pyridine-3-carboxamide,
6-[[(2R)-1-cyclopentyl-2-ethyl-1,2,3,4-tetrahydro-4-methyl-3-oxopyrido[3,4-b]pyrazin-7-yl]amino]-5-methyl-N-(1-methyl-4-piperidinyl)pyridine-3-carboxamide,
1N-cyclopentyl-7-[[5-(4,5-dihydro-4,4-dimethyl-2-oxazolyl)-3-methoxy-2-pyridinyl]amino]-(2R)-ethyl-4N-methyl-1,4-dihydropyrido[3,4-b]pyrazin-3(2H)-one,
N-cyclohexyl-6-[[(2R)-1-cyclopentyl-2-ethyl-1,2,3,4-tetrahydro-4-methyl-3-oxopyrido[3,4-b]pyrazin-7-yl]amino]-5-methoxypyridine-3-carboxamide.

### DEFINITIONS

C₁-C₆-Alkyl-, or a C₁-C₆-alkyl group, is understood to mean a straight-chain or branched, saturated monovalent hydrocarbyl radical, for example a methyl, ethyl, propyl, butyl, pentyl, hexyl, isopropyl, isobutyl, sec-butyl, tert-butyl, isopentyl, 2-methylbutyl, 1-methylbutyl, 1-ethylpropyl, 1,2-dimethylpropyl, neopentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl or 1,2-dimethylbutyl radical. Preferably, C₁-C₆-alkyl-, or a C₁-C₆-alkyl group, is understood to mean C₁-C₄-alkyl- or C₂-C₅-alkyl-, more preferably C₁-C₃-alkyl-, i.e. a methyl, ethyl, propyl or isopropyl radical. C₂-C₄-Alkenyl-, or a C₂-C₄-alkenyl group, is understood to mean a straight-chain or branched, monovalent hydrocarbon radical having one or two C═C double bonds, for example an ethenyl, (E)-prop-2-enyl, (Z)-prop-2-enyl, allyl (prop-1-enyl), allenyl, buten-1-yl or buta-1,3-dienyl radical. Preference is given to ethenyl- and allyl-.

C₂-C₄-Alkynyl, or a C₂-C₄-alkynyl group, is understood to mean a straight-chain or branched, monovalent hydrocarbon radical having one CEC triple bond, for example an ethynyl, propargyl (prop-1-ynyl) or butyn-1-yl radical. Preference is given to ethynyl and propargyl.

C₁-C₄-Alkoxy-, or a C₁-C₄-alkoxy group, is understood to mean a straight-chain or branched, saturated alkyl ether radical —O-alkyl, for example a methoxy, ethoxy, n-propoxy, isopropoxy or tert-butoxy radical.

Preferably, C₁-C₄-alkoxy-, or a C₁-C₄-alkoxy group, is understood to mean C₁-C₃-alkoxy-, more preferably a methoxy or ethoxy radical.

C₁-C₄-Alkylthio-, or a C₁-C₄-alkylthio group, is understood to mean a straight-chain or branched, saturated alkyl thioether radical —S-alkyl, for example a methylthio, ethylthio, n-propylthio, isopropylthio or tert-butylthio radical.

Preferably, C₁-C₄-alkylthio-, or a C₁-C₄-alkylthio group, is understood to mean C₁-C₃-alkylthio-, more preferably a methylthio or ethylthio radical.

A heteroatom is understood to mean —O—, NH—, ═N— or —S—. The heteroatom —NH— may optionally be substituted by C₁-C₃-alkyl, C₁-C₃-alkylcarbonyl, C₁-C₄-alkoxycarbonyl, or —S(═O)₂—C₁-C₃-alkyl.

Preference is given to an oxygen or nitrogen atom.

Oxo, or an oxo substituent, is understood to mean a double-bonded oxygen atom ═O. Oxo may be bonded to atoms of suitable valency, for example to a saturated carbon atom or to sulphur. Preference is given to the bond to carbon to form a carbonyl group —(C═O)—. Preference is further given to the bond of two double-bonded oxygen atoms to sulphur, forming a sulphonyl group —(S═O)₂—.

Halogen is understood to mean fluorine, chlorine, bromine or iodine.

A halo-C₁-C₄-alkyl radical, or halo-C₁-C₄-alkyl-, is understood to mean a C₁-C₄-alkyl radical substituted by at least one halogen substituent, preferably by at least one fluorine substituent. Preference is given to fluoro-C₁-C₃-alkyl radicals, for example difluoromethyl-, trifluoromethyl-, 2,2,2-trifluoroethyl- or pentafluoroethyl-.

Particular preference is given to perfluorinated alkyl radicals such as trifluoromethyl- or pentafluoroethyl-.

Phenyl-C₁-C₃-alkyl- is understood to mean a group composed of an optionally substituted phenyl radical and a C₁-C₃-alkyl group, and bonded to the rest of the molecule via the C₁-C₃-alkyl group.

A halo-C₁-C₄-alkoxy radical, or halo-C₁-C₄-alkoxy-, is understood to mean a C₁-C₄-alkoxy radical substituted by at least one halogen substituent, preferably by at least one fluorine substituent. Preference is given to fluoro-C₁-C₃-alkoxy radicals, for example difluoromethoxy-, trifluoromethoxy- or 2,2,2-trifluoroethoxy-.

A halo-C₁-C₄-alkylthio radical, or halo-C₁-C₄-alkylthio-, is understood to mean a C₁-C₄-alkylthio radical substituted by at least one halogen substituent, preferably by at least one fluorine substituent. Preference is given to fluoro-C₁-C₃-alkylthio radicals, especially trifluoromethylthio-.

A C₁-C₄-alkylcarbonyl radical is understood to mean a C₁-C₄-alkyl-C(═O)— group. Preference is given to acetyl- or propanoyl-.

A C₁-C₄-alkoxycarbonyl radical is understood to mean a C₁-C₄-alkoxy-C(═O)— group. Preference is given to methoxycarbonyl-, ethoxycarbonyl- or tert-butoxycarbonyl-.

A C₁-C₄-alkoxy-C₁-C₄-alkyl radical is understood to mean a C₁-C₄-alkoxy-substituted C₁-C₄-alkyl radical such as, for example, methoxymethyl, methoxyethyl, ethoxymethyl and ethoxyethyl.

Aryl is understood to mean an unsaturated, fully conjugated system which is formed from carbon atoms and has 3, 5 or 7 conjugated double bonds, for example phenyl, naphthyl or phenanthryl. Preference is given to phenyl.

Heteroaryl- is understood to mean ring systems which have an aromatically conjugated ring system and contain at least one and up to five heteroatoms as defined above. These ring systems may have 5, 6 or 7 ring atoms, or else, in the case of fused or benzofused ring systems, combinations of S- and 6-membered ring systems, 5- and 5-membered ring systems, or else 6- and 6-membered ring systems. Examples which may be mentioned are ring systems such as pyrrolyl, pyrazolyl, imidazolyl, triazolyl, tetrazolyl, furanyl, thienyl, oxazolyl, thiazolyl, isoxazolyl, oxadiazolyl, thiadiazolyl, pyridinyl, pyrimidinyl, pyrazinyl, triazinyl, oxazinyl, indolyl, benzimidazolyl, indazolyl, benzotriazolyl, benzothiazolyl, benzoxazolyl, benzofuranyl, benzothienyl, quinolinyl, isoquinolinyl, cinnolinyl, quinazolinyl, quinoxalinyl, imidazopyridylyl or else benzoxazinyl. Preference is given to 5- to 6-membered monocyclic heteroaryl-, for example pyrrolyl-, pyrazolyl-, imidazolyl-, triazolyl-, tetrazolyl-, furanyl-, thienyl-, oxazolyl-, thiazolyl-, isoxazolyl-, oxadiazolyl-, thiadiazolyl-, pyridinyl-, pyrimidinyl-, pyrazinyl-, triazinyl-.

C₃-C₆-Cycloalkyl, C₃-C₈-cycloalkyl, and C₅-C₈-cycloalkyl are understood to mean a monocyclic, saturated ring system formed exclusively from carbon atoms and having, respectively, 3 to 6, 3 to 8, and 5 to 8 atoms. Examples are cyclopropyl-, cyclobutyl-, cyclopentyl-, cyclohexyl-, cycloheptyl- or cyclooctyl-.

C₄-C₆-Cycloalkenyl, C₄-C₈-cycloalkenyl, and C₅-C₈-cycloalkenyl are understood to mean a monocyclic, mono- or polyunsaturated, nonaromatic ring system formed exclusively from carbon atoms and having, respectively, 3 to 6, 3 to 8, and 5 to 8 atoms. Examples are cyclobuten-1-yl-, cyclopenten-1-yl-, cyclohexen-2-yl-, cyclohexen-1-yl- or cycloocta-2,5-dienyl-.

Heterocycloalkyl- is understood to mean a 4- to 8-membered monocyclic, saturated ring system having 1 to 3 heteroatoms as defined above in any combination. Preference is given to 4- to 7-membered heterocycloalkyl groups, particular preference to 5- to 6-membered heterocycloalkyl groups. Examples include pyrrolidinyl-, piperidinyl-, tetrahydrofuranyl-, tetrahydropyranyl-, oxetanyl-, azetidinyl-, azepanyl-, morpholinyl-, thiomorpholinyl- or piperazinyl-.

Heterocycloalkenyl is understood to mean a 4- to 8-membered monocyclic, mono- or polyunsaturated, nonaromatic ring system having 1 to 3 heteroatoms as defined above in any combination. Preference is given to 4- to 7-membered heterocycloalkenyl groups, particular preference to 5- to 6-membered heterocycloalkenyl groups. Examples include 4H-pyranyl-, 2H-pyranyl-, 2,5-dihydro-1H-pyrrolyl-, [1,3]dioxolyl-, 4H-[1,3,4]thiadiazinyl-, 2,5-dihydrofuranyl-, 2,3-dihydrofuranyl-, 2,5-dihydrothiophenyl-, 2,3-dihydrothiophenyl-, 4,5-dihydrooxazolyl-, or 4H-[1,4]thiazinyl-.

C₅-C₁₁-Spirocycloalkyl or C₅-C₁₁-heterospirocycloalkyl where 1 to 4 carbon atoms are replaced by heteroatoms as defined above in any combination is understood to mean a fusion of two saturated ring systems which share one common atom. Examples are spiro[2.2]pentyl-, spiro[2.3]hexyl-, azaspiro[2.3]hexyl-, spiro[3.3]heptyl-, azaspiro[3.3]heptyl-, oxazaspiro[3.3]heptyl-, thiaazaspiro[3.3]heptyl-, oxaspiro[3.3]heptyl-, oxazaspiro[5.3]nonyl-, oxazaspiro[4.3]octyl-, oxazaspiro[5.5]undecyl-, diazaspiro[3.3]heptyl-, thiazaspiro[3.3]heptyl-, thiazaspiro[4.3]octyl-, azaspiro[5.5]decyl-, and the further homologous spiro[3.4], spiro[4.4], spiro[5.5], spiro[6.6], spiro[2.4], spiro[2.5], spiro[2.6], spiro[3.5], spiro[3.6], spiro[4.5], spiro[4.6] and spiro[5.6] systems including the variants modified by heteroatoms as per the definition. Preference is given to C₆-C₈-heterospirocycloalkyl.

C₆-C₁₂-Bicycloalkyl or C₆-C₁₂-heterobicycloalkyl where 1 to 4 carbon atoms are replaced by heteroatoms as defined above in any combination is understood to mean a fusion of two saturated ring systems which share two directly adjacent atoms. Examples are bicyclo[2.2.0]hexyl, bicyclo[3.3.0]octyl, bicyclo[4.4.0]decyl, bicyclo[5.4.0]undecyl, bicyclo[3.2.0]heptyl, bicyclo[4.2.0]octyl, bicyclo[5.2.0]nonyl, bicyclo[6.2.0]decyl, bicyclo[4.3.0]nonyl, bicyclo[5.3.0]decyl, bicyclo[6.3.0]undecyl and bicyclo[5.4.0]undecyl, including the variants modified by heteroatoms, for example azabicyclo[3.3.0]octyl, azabicyclo[4.3.0]nonyl, diazabicyclo[4.3.0]nonyl, oxazabicyclo[4.3.0]nonyl, thiazabicyclo[4.3.0]nonyl or azabicyclo[4.4.0]decyl, and the further possible combinations as per the definition. Preference is given to C₆-C₁₀-heterobicycloalkyl.

A bridged C₆-C₁₂ ring system such as bridged C₆-C₁₂-cycloalkyl or bridged C₆-C₁₂-heterocycloalkyl is understood to mean a fusion of at least two saturated rings which share two atoms that are not directly adjacent to one another. This may give rise either to a bridged carbocycle (bridged cycloalkyl) or to a bridged heterocycle (bridged heterocycloalkyl) where 1 to 4 carbon atoms are replaced by heteroatoms as defined above in any combination. Examples are bicyclo[2.2.1]heptyl-, azabicyclo[2.2.1]heptyl-, oxazabicyclo[2.2.1]heptyl-, thiazabicyclo[2.2.1]heptyl-, diazabicyclo[2.2.1]heptyl-, bicyclo[2.2.2]octyl-, azabicyclo[2.2.2]octyl-, diazabicyclo[2.2.2]octyl-, oxazabicyclo[2.2.2]octyl-, thiazabicyclo[2.2.2]octyl-, bicyclo[3.2.1]octyl-, azabicyclo[3.2.1]octyl-, diazabicyclo[3.2.1]octyl-, oxazabicyclo[3.2.1]octyl thiazabicyclo[3.2.1]octyl-, bicyclo[3.3.1]nonyl-, azabicyclo[3.3.1]nonyl diazabicyclo[3.3.1]nonyl-, oxazabicyclo[3.3.1]nonyl-, thiazabicyclo[3.3.1]nonyl-, bicyclo[4.2.1]nonyl-, azabicyclo[4.2.1]nonyl-, diazabicyclo[4.2.1]nonyl-, oxazabicyclo[4.2.1]nonyl-, thiazabicyclo[4.2.1]nonyl-, bicyclo[3.3.2]decyl-, azabicyclo[3.3.2]decyl diazabicyclo[3.3.2]decyl oxazabicyclo[3.3.2]decyl-, thiazabicyclo[3.3.2]decyl- or azabicyclo[4.2.2]decyl- and the further possible combinations as per the definition. Preference is given to bridged C₆-C₁₀-heterocycloalkyl.

Compounds according to the invention are the compounds of the general formula (I) and the salts, solvates and solvates of the salts thereof, the compounds, encompassed by the general formula (I), of the formulae specified hereinafter and the salts, solvates and solvates of the salts thereof, and the compounds encompassed by the general formula (I) and specified hereinafter as working examples and the salts, solvates and solvates of the salts thereof, to the extent that the compounds encompassed by the general formula (I) and specified hereinafter are not already salts, solvates and solvates of the salts.

The present invention is likewise considered to encompass the use of the salts of the compounds according to the invention.

Preferred salts in the context of the present invention are physiologically acceptable salts of the compounds according to the invention. However, the invention also encompasses salts which themselves are unsuitable for pharmaceutical applications but which can be used, for example, for the isolation or purification of the compounds according to the invention.

Physiologically acceptable salts of the compounds according to the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

The present invention furthermore provides all the possible crystalline and polymorphous forms of the compounds according to the invention, where the polymorphs may be present either as single polymorphs or as a mixture of a plurality of polymorphs in all concentration ranges.

The present invention also relates to medicaments comprising the compounds according to the invention together with at least one or more further active compounds, especially for prophylaxis and/or treatment of neoplastic disorders.

Solvates in the context of the invention are described as those forms of the compounds according to the invention which form a complex in the solid or liquid state by coordination with solvent molecules. Hydrates are a specific form of the solvates in which the coordination is with water. Solvates preferred in the context of the present invention are hydrates.

The compounds according to the invention may, depending on their structure, exist in different stereoisomeric forms, i.e. in the form of configurational isomers or else optionally as conformational isomers. The compounds according to the invention may have a centre of asymmetry at the carbon atom to which R⁵ and R⁶ are attached. They may therefore take the form of pure enantiomers, racemates, or else of diastereomers or mixtures thereof when one or more of the substituents described in the formula (I) contains a further element of asymmetry, for example a chiral carbon atom. The present invention therefore also encompasses diastereomers and the respective mixtures thereof. The pure stereoisomers can be isolated from such mixtures in a known manner; chromatography processes are preferably used for this, in particular HPLC chromatography on a chiral or achiral phase.

In general, the enantiomers according to the invention inhibit the target proteins to different degrees and have different activity in the cancer cell lines studied. The more active enantiomer is preferred, which is often that in which the centre of asymmetry represented by the carbon atom bonded to R⁵ and R⁶ has (R) configuration.

If the compounds according to the invention can occur in tautomeric forms, the present invention encompasses all the tautomeric forms.

The present invention also encompasses all suitable isotopic variants of the compounds according to the invention. An isotopic variant of a compound according to the invention is understood here as meaning a compound in which at least one atom within the compound according to the invention has been exchanged for another atom of the same atomic number, but with a different atomic mass than the atomic mass which usually or predominantly occurs in nature. Examples of isotopes which can be incorporated into a compound according to the invention are those of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of a compound according to the invention, especially those in which one or more radioactive isotopes have been incorporated, may be beneficial, for example, for the examination of the mechanism of action or of the active ingredient distribution in the body; due to comparatively easy preparability and detectability, especially compounds labelled with ³H or ¹⁴C isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required; such modifications of the compounds according to the invention may therefore in some cases also constitute a preferred embodiment of the present invention. Isotopic variants of the compounds according to the invention can be prepared by the processes known to those skilled in the art, for example by the methods described further below and the procedures described in the working examples, by using corresponding isotopic modifications of the respective reagents and/or starting compounds.

The present invention moreover also includes prodrugs of the compounds according to the invention. The term “prodrugs” encompasses compounds which for their part may be biologically active or inactive but are converted during their residence time in the body into compounds according to the invention (for example by metabolism or hydrolysis).

The compounds according to the invention can act systemically and/or locally. For this purpose, they can be administered in a suitable manner, for example by the oral, parenteral, pulmonary, nasal, sublingual, lingual, buccal, rectal, dermal, transdermal, conjunctival or otic route, or as implant or stent.

The compounds according to the invention can be administered in administration forms suitable for these administration routes.

Suitable administration forms for oral administration are those which function according to the prior art and deliver the compounds according to the invention rapidly and/or in modified fashion, and which contain the compounds according to the invention in crystalline and/or amorphized and/or dissolved form, for example tablets (uncoated or coated tablets, for example having enteric coatings or coatings which are insoluble or dissolve with a delay and control the release of the compound according to the invention), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can bypass an absorption step (for example intravenously, intraarterially, intracardially, intraspinally or intralumbally) or include an absorption (for example intramuscularly, subcutaneously, intracutaneously, percutaneously or intraperitoneally). Administration forms suitable for parenteral administration include preparations for injection and infusion in the form of solutions, suspensions, emulsions, lyophilizates or sterile powders.

Suitable administration forms for the other administration routes are, for example, pharmaceutical forms for inhalation (including powder inhalers, nebulizers), nasal drops, solutions or sprays; tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, transdermal therapeutic systems (for example patches), milk, pastes, foams, dusting powders, implants or stents.

The compounds according to the invention can be converted to the administration forms mentioned. This can be accomplished in a manner known per se by mixing with inert, nontoxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), colourants (e.g. inorganic pigments, for example iron oxides) and flavour and/or odour correctants.

The present invention furthermore provides medicaments which comprise the compounds according to the invention, typically together with one or more inert, nontoxic, pharmaceutically suitable auxiliaries, and the use thereof for the aforementioned purposes.

The compounds according to the invention are formulated to give pharmaceutical preparations in a manner known per se, by converting the active compound(s) to the desired administration form with the excipients customary in the pharmaceutical formulation.

The excipients used may, for example, be carrier substances, fillers, disintegrants, binders, humectants, glidants, absorbents and adsorbents, diluents, solvents, cosolvents, emulsifiers, solubilizers, taste correctors, colourants, preservatives, stabilizers, wetting agents, salts for modifying the osmotic pressure or buffers. Reference should be made to Remington's Pharmaceutical Science, 15th ed. Mack Publishing Company, East Pennsylvania (1980).

The pharmaceutical formulations may be in solid form, for example in the form of tablets, coated tablets, pills, suppositories, capsules, transdermal systems, or in semisolid form, for example in the form of ointments, creams, gels, suppositories, emulsions, or in liquid form, for example in the form of solutions, tinctures, suspensions or emulsions.

Excipients in the context of the invention may, for example, be salts, saccharides (mono-, di-, tri-, oligo- and/or polysaccharides), proteins, amino acids, peptides, fats, waxes, oils, hydrocarbons and derivatives thereof, and the excipients may be of natural origin or be obtained by synthetic or partially synthetic means.

Useful forms for oral or peroral administration are especially tablets, sugar-coated tablets, capsules, pills, powders, granules, pastilles, suspensions, emulsions or solutions.

Useful forms for parenteral administration are especially suspensions, emulsions, and particularly solutions.

The compounds according to the invention are suitable for prophylaxis and/or treatment of hyperproliferative disorders, for example psoriasis, keloids and other hyperplasias which affect the skin, benign prostate hyperplasias (BPH), solid tumours and haematological tumours.

Solid tumours that can be treated in accordance with the invention are, for example, tumours of the breast, the respiratory tract, the brain, the reproductive organs, the gastrointestinal tract, the urogenital tract, the eye, the liver, the skin, the head and the neck, the thyroid gland, the parathyroid gland, the bones, and the connective tissue and metastases of these tumours.

Haematological tumours that can be treated are, for example, multiple myeloma, lymphoma or leukaemia.

Breast tumours that can be treated are, for example, mammary carcinoma with positive hormone receptor status, mammary carcinoma with negative hormone receptor status, Her-2-positive mammary carcinoma, hormone receptor- and Her-2-negative mammary carcinoma, BRCA-associated mammary carcinoma and inflammatory mammary carcinoma.

Tumours of the respiratory tract that can be treated are, for example, non-small-cell bronchial carcinoma and small-cell bronchial carcinoma.

Brain tumours that can be treated are, for example, glioma, glioblastoma, astrocytoma, meningioma and medulloblastoma.

Tumours of the male reproductive organs that can be treated are, for example, prostate carcinoma, malignant epididymal tumours, malignant testicular tumours and penile carcinoma.

Tumours of the female reproductive organs that can be treated are, for example, endometrial carcinoma, cervical carcinoma, ovarian carcinoma, vaginal carcinoma and vulvar carcinoma.

Tumours of the gastrointestinal tract that can be treated are, for example, colorectal carcinoma, anal carcinoma, gastric carcinoma, pancreatic carcinoma, oesophageal carcinoma, gallbladder carcinoma, small-intestinal carcinoma, salivary gland carcinoma, neuroendocrine tumours and gastrointestinal stromal tumours.

Tumours of the urogenital tract that can be treated are, for example, urinary bladder carcinoma, renal cell carcinoma, and carcinoma of the renal pelvis and of the urinary tract.

Tumours of the eye that can be treated are, for example, retinoblastoma and intraocular melanoma.

Tumours of the liver that can be treated are, for example, hepatocellular carcinoma and cholangiocellular carcinoma.

Tumours of the skin that can be treated are, for example, malignant melanoma, basalioma, spinalioma, Kaposi's sarcoma and Merkel cell carcinoma.

Tumours of the head and neck that can be treated are, for example, laryngeal carcinoma and carcinoma of the pharynx and of the oral cavity.

Sarcomas that can be treated are, for example, soft tissue sarcoma and osteosarcoma.

Lymphomas that can be treated are, for example, non-Hodgkin's lymphoma, Hodgkin's lymphoma, cutaneous lymphoma, lymphoma of the central nervous system and AIDS-associated lymphoma.

Leukaemias that can be treated are, for example, acute myeloid leukaemia, chronic myeloid leukaemia, acute lymphatic leukaemia, chronic lymphatic leukaemia and hair cell leukaemia.

Advantageously, the compounds according to the invention can be used for prophylaxis and/or treatment of leukaemia, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

The present application furthermore provides the compounds according to the invention for prophylaxis and/or therapy of leukaemias, especially acute myeloid leukaemias, prostate carcinomas, especially androgen receptor-positive prostate carcinomas, mammary carcinomas, especially oestrogen receptor alpha-negative mammary carcinomas, melanomas or multiple myelomas.

The compounds according to the invention are also suitable for prophylaxis and/or treatment of benign hyperproliferative diseases, for example endometriosis, leiomyoma and benign prostate hyperplasia.

The compounds according to the invention are also suitable for prophylaxis and/or treatment of systemic inflammatory diseases, especially LPS-induced endotoxic shock and/or bacteria-induced sepsis.

The compounds according to the invention are also suitable for prophylaxis and/or treatment of inflammatory or autoimmune disorders, for example:
pulmonary disorders associated with inflammatory, allergic and/or proliferative processes: chronic obstructive pulmonary disorders of any origin, particularly bronchial asthma; bronchitis of different origin; all forms of restrictive pulmonary disorders, particularly allergic alveolitis; all forms of pulmonary oedema, particularly toxic pulmonary oedema; sarcoidoses and granulomatoses, particularly Boeck's disease,
rheumatic disorders/autoimmune disorders/joint disorders associated with inflammatory, allergic and/or proliferative processes: all forms of rheumatic disorders, especially rheumatoid arthritis, acute rheumatic fever, polymyalgia rheumatica; reactive arthritis; inflammatory soft-tissue disorders of other origin; arthritic symptoms in the case of degenerative joint disorders (arthroses); traumatic arthritides; collagenoses of any origin, e.g. systemic lupus erythematosus, scleroderma, polymyositis, dermatomyositis, Sjögren's syndrome, Still's syndrome, Felty's syndrome
allergies associated with inflammatory and/or proliferative processes: all forms of allergic reactions, e.g. angiooedema, hay fever, insect bites, allergic reactions to medicaments, blood derivatives, contrast agents, etc., anaphylactic shock, urticaria, contact dermatitis
vascular inflammation (vasculitis): panarteritis nodosa, temporal arteritis, erythema nodosum
dermatological disorders associated with inflammatory, allergic and/or proliferative processes: atopic dermatitis; psoriasis; pityriasis rubra pilaris; erythematous disorders triggered by different noxae, for example radiation, chemicals, burns, etc.; bullous dermatoses; lichenoid disorders; pruritus; seborrhoeic eczema; rosacea; pemphigus vulgaris; erythema exsudativum multiforme; balanitis; vulvitis; hair loss, such as alopecia areata; cutaneous T-cell lymphoma
renal disorders associated with inflammatory, allergic and/or proliferative processes: nephrotic syndrome; all nephritides,
hepatic disorders associated with inflammatory, allergic and/or proliferative processes: acute hepatic disintegration; acute hepatitis of different origin, for example viral, toxic, medicament-induced; chronic aggressive and/or chronic intermittent hepatitis
gastrointestinal disorders associated with inflammatory, allergic and/or proliferative processes: regional enteritis (Crohn's disease); ulcerative colitis; gastritis; reflux oesophagitis; gastroenteritides of other origin, e.g. indigenous sprue
proctological disorders associated with inflammatory, allergic and/or proliferative processes: anal eczema; fissures; haemorrhoids; idiopathic proctitis
ocular disorders associated with inflammatory, allergic and/or proliferative processes: allergic keratitis, uveitis, iritis; conjunctivitis; blepharitis; optic neuritis; chlorioditis; sympathetic ophthalmia
disorders of the ear-nose-throat region associated with inflammatory, allergic and/or proliferative processes: allergic rhinitis, hay fever; otitis externa, for example caused by contact eczema, infection, etc.; otitis media
neurological disorders associated with inflammatory, allergic and/or proliferative processes: cerebral oedema, particularly tumour-related cerebral oedema; multiple sclerosis; acute encephalomyelitis; meningitis; various forms of seizure, for example West's syndrome
haematological disorders associated with inflammatory, allergic and/or proliferative processes: congenital haemolytic anaemia; idiopathic thrombocytopenia,
neoplastic disorders associated with inflammatory, allergic and/or proliferative processes: acute lymphatic leukaemia; malignant lymphoma; lymphogranulomatoses; lymphosarcoma; extensive metastases, particularly in the case of mammary, bronchial and prostate carcinoma
endocrine disorders associated with inflammatory, allergic and/or proliferative processes: endocrine orbitopathy; thyrotoxic crisis; de Quervain's thyroiditis; Hashimoto's thyroiditis; Basedow's disease,
organ and tissue transplants, graft-versus-host disease,
severe states of shock, for example anaphylactic shock, systemic inflammatory response syndrome (SIRS)
substitution therapy in the case of: congenital primary renal insufficiency, for example congenital adrenogenital syndrome; acquired primary renal insufficiency, for example Addison's disease, autoimmune adrenalitis, postinfectious tumours, metastases, etc; congenital secondary renal insufficiency, for example congenital hypopituitarism; acquired secondary renal insufficiency, for example postinfectious, tumours, etc.
emesis associated with inflammatory, allergic and/or proliferative processes, for example in combination with a 5-HT3 antagonist in the case of cytostatic-induced vomiting
pain of inflammatory origin, for example lumbago.

The compounds according to the invention are also suitable for the treatment of viral disorders, for example infections caused by papilloma viruses, herpes viruses, Epstein-Barr viruses, hepatitis B or C viruses, and human immunodeficiency viruses.

The compounds according to the invention are also suitable for the treatment of atherosclerosis, dyslipidaemia, hypercholesterolaemia, hypertriglyceridaemia, peripheral vascular disorders, cardiovascular disorders, angina pectoris, ischaemia, stroke, myocardial infarction, angioplastic restenosis, hypertension, thrombosis, obesity, endotoxaemia.

The compounds according to the invention are also suitable for the treatment of neurodegenerative diseases, for example multiple sclerosis, Alzheimer's disease and Parkinson's disease.

These disorders are well characterized in man, but also exist in other mammals.

The present application furthermore provides the compounds according to the invention for use as medicaments, in particular for the prophylaxis and/or therapy of tumour disorders.

The present application furthermore provides the compounds according to the invention for prophylaxis and/or therapy of leukaemia, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

The present application furthermore provides the compounds according to the invention for prophylaxis and/or therapy of leukaemias, especially acute myeloid leukaemias, prostate carcinomas, especially androgen receptor-positive prostate carcinomas, mammary carcinomas, especially oestrogen receptor alpha-negative mammary carcinomas, melanomas or multiple myelomas.

The invention furthermore provides for the use of the compounds according to the invention for production of a medicament.

The present application furthermore provides for the use of the compounds according to the invention for production of a medicament for prophylaxis and/or therapy of neoplastic disorders.

The present application furthermore provides for the use of the compounds according to the invention for production of a medicament for prophylaxis and/or therapy of leukaemias, especially acute myeloid leukaemias, prostate carcinomas, especially androgen receptor-positive prostate carcinomas, cervical carcinomas, mammary carcinomas, especially hormone receptor-negative, hormone receptor-positive or BRCA-associated mammary carcinomas, pancreatic carcinomas, renal cell carcinomas, hepatocellular carcinomas, melanomas and other skin tumours, non-small-cell bronchial carcinomas, endometrial carcinomas and colorectal carcinomas.

The present application furthermore provides for the use of the compounds according to the invention for production of a medicament for prophylaxis and/or therapy of leukaemias, especially acute myeloid leukaemias, prostate carcinomas, especially androgen receptor-positive prostate carcinomas, mammary carcinomas, especially oestrogen receptor alpha-negative mammary carcinomas, melanomas or multiple myelomas.

The present application furthermore provides for the use of the compounds according to the invention for prophylaxis and/or therapy of neoplastic disorders.

The present application furthermore provides for the use of the compounds according to the invention for prophylaxis and/or therapy of leukaemias, especially acute myeloid leukaemias, prostate carcinomas, especially androgen receptor-positive prostate carcinomas, cervical carcinomas, mammary carcinomas, especially hormone receptor-negative, hormone receptor-positive or BRCA-associated mammary carcinomas, pancreatic carcinomas, renal cell carcinomas, hepatocellular carcinomas, melanomas and other skin tumours, non-small-cell bronchial carcinomas, endometrial carcinomas and colorectal carcinomas.

The present application furthermore provides for the use of the compounds according to the invention for prophylaxis and/or therapy of leukaemias, especially acute myeloid leukaemias, prostate carcinomas, especially androgen receptor-positive prostate carcinomas, mammary carcinomas, especially oestrogen receptor alpha-negative mammary carcinomas, melanomas or multiple myelomas.

The present application furthermore provides pharmaceutical formulations in the form of tablets comprising one of the compounds according to the invention for prophylaxis and/or therapy of leukaemias, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

The present application furthermore provides pharmaceutical formulations in the form of tablets comprising one of the compounds according to the invention for prophylaxis and/or therapy of leukaemias, especially acute myeloid leukaemias, prostate carcinomas, especially androgen receptor-positive prostate carcinomas, mammary carcinomas, especially oestrogen receptor alpha-negative mammary carcinomas, melanomas or multiple myelomas.

The invention furthermore provides for the use of the compounds according to the invention for treatment of disorders associated with proliferative processes.

The invention furthermore provides for the use of the compounds according to the invention for treatment of benign hyperplasias, inflammation disorders, autoimmune disorders, sepsis, viral infections, vascular disorders and neurodegenerative disorders.

The compounds according to the invention can be used alone or, if required, in combination with one or more further pharmacologically active substances, provided that this combination does not lead to undesirable and unacceptable side effects. The present invention therefore further provides medicaments comprising a compound according to the invention and one or more further active compounds, especially for prophylaxis and/or treatment of the aforementioned disorders.

For example, the compounds according to the invention can be combined with known antihyperproliferative, cytostatic or cytotoxic chemical and biological substances for treatment of cancer. The combination of the compounds according to the invention with other substances commonly used for cancer treatment, or else with radiotherapy, is particularly appropriate.

An illustrative but nonexhaustive list of active compounds suitable for combinations is as follows:

abiraterone acetate, abraxane, acolbifene, Actimmune, actinomycin D (dactinomycin), afatinib, affinitak, Afinitor, aldesleukin, alendronic acid, alfaferone, alitretinoin, allopurinol, Aloprim, Aloxi, alpharadin, altretamine, aminoglutethimide, aminopterin, amifostine, amrubicin, amsacrine, anastrozole, anzmet, apatinib, Aranesp, arglabin, arsenic trioxide, Aromasin, arzoxifen, asoprisnil, L-asparaginase, atamestane, atrasentane, avastin, axitinib, 5-azacytidine, azathioprine, BCG or Tice BCG, bendamustine, bestatin, beta-methasone acetate, betamethasone sodium phosphate, bexarotene, bicalutamide, bleomycin sulphate, broxuridine, bortezomib, bosutinib, busulfan, cabazitaxel, calcitonin, campath, camptothecin, capecitabine, carboplatin, carfilzomib, carmustine, casodex, CCI-779, CDC-501, cediranib, cefesone, celebrex, celmoleukin, cerubidine, cediranib, chlorambucil, cisplatin, cladribine, clodronic acid, clofarabine, colaspase, corixa, crisnatol, crizotinib, cyclophosphamide, cyproterone acetate, cytarabine, dacarbazine, dactinomycin, dasatinib, daunorubicin, DaunoXome, Decadron, Decadron Phosphate, decitabine, degarelix, delestrogen, denileukin diftitox, depomedrol, deslorelin, dexrazoxane, diethylstilbestrol, diflucan, 2′,2′-difluorodeoxycytidine, DN-101, docetaxel, doxifluridine, doxorubicin (Adriamycin), dronabinol, dSLIM, dutasteride, DW-166HC, edotecarin, eflornithine, Eligard, Elitek, Ellence, Emend, enzalutamide, epirubicin, epoetin-alfa, Epogen, epothilone and derivatives thereof, eptaplatin, ergamisol, erlotinib, erythro-hydroxynonyladenine, estrace, oestradiol, oestramustine sodium phosphate, ethinyloestradiol, Ethyol, etidronic acid, etopophos, etoposide, everolimus, exatecan, exemestane, fadrozole, farston, fenretinide, filgrastim, finasteride, fligrastim, floxuridine, fluconazole, fludarabine, 5-fluorodeoxyuridine monophosphate, 5-fluorouracil (5-FU), fluoxymesterone, flutamide, folotin, formestane, fosteabine, fotemustine, fulvestrant, Gammagard, gefitinib, gemcitabine, gemtuzumab, Gleevec, Gliadel, goserelin, gossypol, granisetron hydrochloride, hexamethylmelamine, histamine dihydrochloride, histrelin, holmium-166-DOTPM, hycamtin, hydrocortone, erythro-hydroxynonyladenine, hydroxyurea, hydroxyprogesterone caproate, ibandronic acid, ibritumomab tiuxetan, idarubicin, ifosfamide, imatinib, iniparib, interferon-alpha, interferon-alpha-2, interferon-alpha-2α, interferon-alpha-2β, interferon-alpha-n1, interferon-alpha-n3, interferon-beta, interferon-gamma-1α, interleukin-2, intron A, iressa, irinotecan, ixabepilone, keyhole limpet haemocyanin, kytril, lanreotide, lapatinib, lasofoxifene, lenalidomide, lentinan sulphate, lestaurtinib, letrozole, leucovorin, leuprolide, leuprolide acetate, levamisole, levofolic acid calcium salt, levothroid, levoxyl, Libra, liposomal MTP-PE, lomustine, lonafarnib, lonidamine, marinol, mechlorethamine, mecobalamine, medroxyprogesterone acetate, megestrol acetate, melphalan, Menest, 6-mercaptopurine, mesna, methotrexate, metvix, miltefosine, minocycline, minodronate, miproxifen, mitomycin C, mitotan, mitoxantrone, modrenal, MS-209, MX-6, myocet, nafarelin, nedaplatin, nelarabine, nemorubicin, neovastat, neratinib, neulasta, neumega, neupogen, nilotimib, nilutamide, nimustine, nolatrexed, nolvadex, NSC-631570, obatoclax, oblimersen, OCT-43, octreotide, olaparib, ondansetron hydrochloride, Onco-TCS, Orapred, Osidem, oxaliplatin, paclitaxel, pamidronate disodium, pazopanib, pediapred, pegaspargase, pegasys, pemetrexed, pentostatin, N-phosphonoacetyl-L-aspartate, picibanil, pilocarpine hydrochloride, pirarubicin, plerixafor, plicamycin, PN-401, porfimer sodium, prednimustine, prednisolone, prednisone, Premarin, procarbazine, Procrit, QS-21, quazepam, R-1589, raloxifene, raltitrexed, ranpirnas, RDEA119, Rebif, regorafenib, 13-cis-retinoic acid, rhenium-186 etidronate, rituximab, roferon-A, romidepsin, romurtide, ruxolitinib, salagen, salinomycin, sandostatin, sargramostim, satraplatin, semaxatinib, semustine, seocalcitol, sipuleucel-T, sizofiran, sobuzoxan, Solu-Medrol, sorafenib, streptozocin, strontium-89 chloride, sunitinib, Synthroid, T-138067, tamoxifen, tamsulosin, Tarceva, tasonermin, tastolactone, Taxoprexin, Taxoter, teceleukin, temozolomide, temsirolimus, teniposide, testosterone propionate, Testred, thalidomide, thymosin alpha-1, thioguanine, thiotepa, thyrotropin, tiazorufin, tiludronic acid, tipifarnib, tirapazamine, TLK-286, toceranib, topotecan, toremifen, tositumomab, tastuzumab, teosulfan, transMID-107R, tretinoin, Trexall, trimethylmelamine, trimetrexate, triptorelin acetate, triptorelin pamoate, trofosfamide, UFT, uridine, valrubicin, valspodar, vandetanib, vapreotide, vatalanib, vemurafinib, verte-porfin, vesnarinone, vinblastine, vincristine, vindesine, vinflumine, vinorelbine, virulizin, vismodegib, Xeloda, Z-100, Zinecard, zinostatin stimalamer, zofran, zoledronic acid.

More particularly, the compounds according to the invention can be combined with antibodies, for example aflibercept, alemtuzumab, bevacizumab, brentuximumab, catumaxomab, cetuximab, denosumab, edrecolomab, gemtuzumab, ibritumomab, ipilimumab, ofatumumab, panitumumab, pertuzumab, rituximab, tositumumab or trastuzumab, and also with recombinant proteins.

More particularly, the compounds according to the invention can be used in combination with treatments directed against angiogenesis, for example bevacizumab, axitinib, regorafenib, cediranib, sorafenib, sunitinib, lenalidomide or thalidomide.

Combinations with antihormones and steroidal metabolic enzyme inhibitors are particularly suitable because of their favourable profile of side effects.

Combinations with P-TEFb inhibitors and CDK9 inhibitors are likewise particularly suitable because of the possible synergistic effects.

Generally, the following aims can be pursued with the combination of the compounds according to the invention with other cytostatically or cytotoxically active agents:
improved efficacy in slowing the growth of a tumour, in reducing its size or even in completely eliminating it, compared with treatment with an individual active ingredient;
the possibility of using the chemotherapeutics used in a lower dosage than in the case of monotherapy;
the possibility of a more tolerable therapy with fewer side effects compared with individual administration;
the possibility of treatment of a broader spectrum of neoplastic disorders;
the achievement of a higher rate of response to the therapy;
a longer survival time of the patient compared with present-day standard therapy.

In addition, the compounds according to the invention can also be used in conjunction with radiotherapy and/or surgical intervention.

### Preparation of the Compounds According to the Invention

In the present description:

NMR signals are reported with their respective recognizable multiplicities or combinations thereof. In this context, s=singlet, d=doublet, t=triplet, q=quartet, qi=quintet, sp=septet, m=multiplet, b=broad signal. Signals having combined multiplicities are reported, for example, as dd=doublet of doublets.
CDCl₃ deuterochloroform
dba dibenzylideneacetone
DMF N,N-dimethylformamide
DMSO-d6 deuterated dimethyl sulphoxide
DMSO dimethyl sulphoxide
HATU (7-aza-1H-benzotriazol-1-yl)-1,1,3,3-tetramethyluronium hexafluorophosphate
RP-HPLC reverse-phase high-pressure liquid chromatography
RT room temperature
THF tetrahydrofuran
HBTU O-benzotriazole-N,N,N′,N′-tetramethyluronium hexafluorophosphate
PyBOB (benzotriazol-1-yl)oxytripyrrolidinophosphonium hexafluorophosphate
T3P 2,4,6-tripropyl-1,3,5,2,4,6-trioxatriphosphorinane 2,4,6-trioxide
LCMS liquid chromatography coupled with mass spectrometry
CHAPS 3-{dimethyl[3-(4-{5,9,16-trihydroxy-2,15-dimethyltetracyclo-[8.7.0.0^{2,7}.0^{11,15}]heptadecan-14-yl}pentanamido)propyl]-azaniumyl}propane-1-sulphonate
(+)-BINAP (R)-(+)-2,2′bis(diphenylphosphino)-1,1′-binaphthyl
(±)-BINAP 2,2′-bis(diphenylphosphino)-1,1′-binaphthyl (racemic)
TBTU (benzotriazol-1-yloxy)bisdimethylaminomethylium fluoroborate
DCC dicyclohexylcarbodiimide

### General Description of the Preparation of the Compounds of the General Formula (I) According to the Invention

The compounds of the formulae (Ia), (Ib) and (Ic) according to the invention shown in Scheme 1 can be prepared via synthesis routes described hereinafter. The formulae specified represent different portions of the general formula (I) in which A, R², R³, R⁴, R⁵, R⁶, R⁷, R⁸, R⁹ and n are each as defined for the general formula (I). In carboxamides of the formula (Ia), a —C(═O)NR⁸R⁹ group is at the position of R¹; in sulphonamides of the formula (Ib), —S(═O)₂NR⁸R⁹ is at the position of R¹, and in compounds (Ic), finally, HetAr, which is 5-membered monocyclic heteroaryl- as defined in formula (I) for R¹, is at the position of R¹.

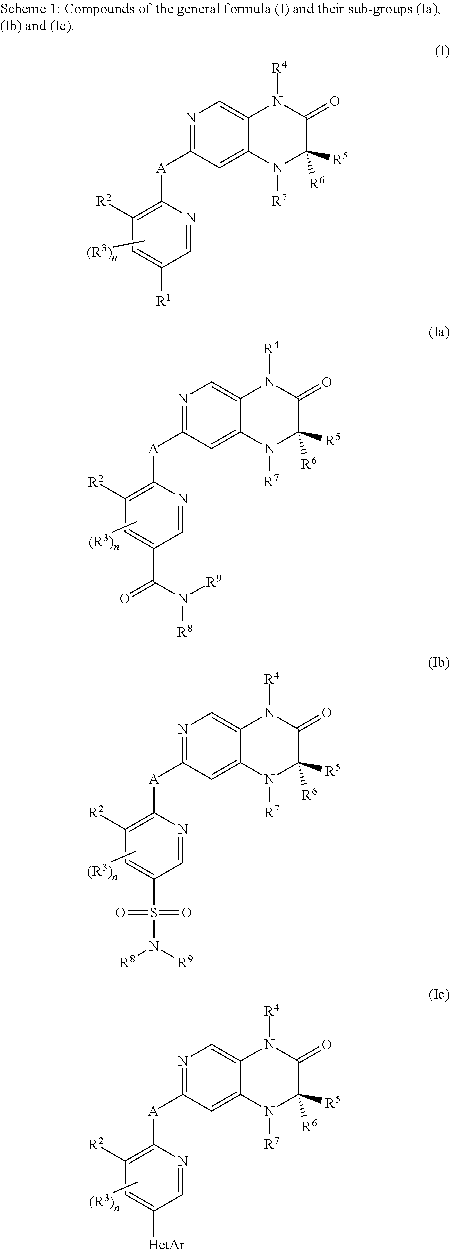

In addition to the synthesis sequences discussed hereinafter, it is also possible, in accordance with the general knowledge of the person skilled in the art in organic chemistry, to take other synthesis routes for the synthesis of compounds of the general formula (I) according to the invention. The sequence of the synthesis steps shown in the schemes which follow is not binding, and synthesis steps from various of the schemes shown hereinafter may optionally be combined to form new sequences. In addition, interconversions of the substituents R², R³, R⁴, R⁵, R⁶, R⁷, R⁸, R⁹ can be performed before or after the synthesis stages shown. Examples of such conversions are the introduction or elimination of protective groups, reduction or oxidation of functional groups, halogenation, metallation, metal-catalysed coupling reactions, substitution reactions or further reactions known to the person skilled in the art. These reactions include conversions which introduce a functional group which enables the further conversion of substituents. Suitable protecting groups and methods for the introduction and removal thereof are known to the person skilled in the art (see, for example, T. W. Greene and P. G. M. Wuts in: Protective Groups in Organic Synthesis, 3rd Edition, Wiley 1999). In addition, it is possible to combine two or more reaction steps without intermediate workup in a manner known to the person skilled in the art (for example in what are called “one-pot” reactions).

Scheme 2 illustrates the construction of amides of the formula (V) from simple pyridine derivatives such as 5-amino-2,4-dichloropyridine ((II), CAS-No. 7321-93-9). For the preparation of (III) from (II), it is possible to use a multitude of methods for preparing amides from the azidocarboxylic acids of the formula (IIa) in which R⁵ and R⁶ are each as defined for the general formula (I). Thus, it is possible to use coupling reagents known to the person skilled in the art, such as TBTU, HATU or DCC. Likewise suitable is the reaction of the azidocarboxylic acids used with an inorganic acid chloride such as thionyl chloride, phosphorus oxychloride or oxalyl chloride, followed by addition of the pyridineamine. The preparation of the azidocarboxylic acids required is described in the literature (Chem Eur J (2010), 16, p 7572 ff, D. Tietze et al.; J Org Chem (2010), 75, p 6532ff, Katritzky et al.). The carboxylic acid azides have to be handled very carefully as they may decompose explosively. Also, storage of the reagents required for introducing the azide should be dispensed with. These aspects are discussed in Katritzky et al.

To reduce the azido group in (III), which leads to amines of the formula (IV), the reaction with trialkyl- or triarylphosphines can be conducted according to Staudinger (Tetrahedron (2012), 68, p 697ff, Laschat et al.). An example of a suitable phosphine is trimethylphosphine. The amines (IV) can be isolated as the free base or, advantageously, in salt form, for instance as the hydrochloride. To this end, the crude amine of the formula (IV) is dissolved in a nonpolar solvent, for example diethyl ether, and precipitated as salt by addition of an acid, for example hydrogen chloride. The further conversion to compounds of the formula (V) with introduction of the R⁷ radical, which is as defined for the general formula (I), can preferably be conducted via the reductive amination known to the person skilled in the art (for representative procedures see, for example, US2010/105906 A1). This involves reacting the primary amine (IV), as the free base or in salt form, in situ with an aldehyde or ketone suitable for the introduction of R⁷ to give an imine, and then transforming the latter by addition of a suitable reducing agent such as sodium triacetoxyborohydride to give the secondary amine of the formula (V).

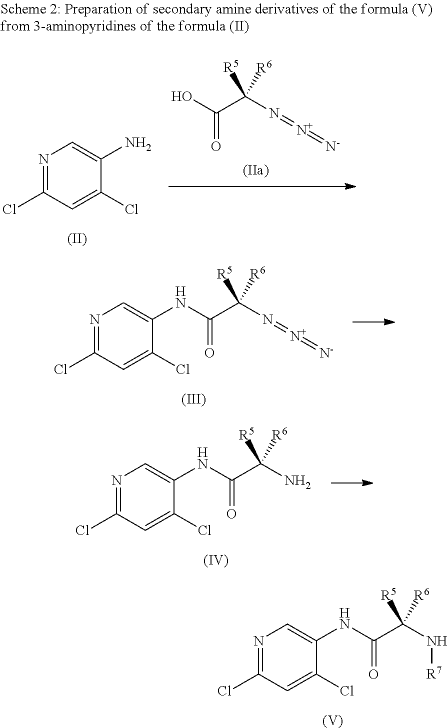

An alternative access to intermediates of the formula (V) in which R⁵, R⁶ and R⁷ are defined as in the general formula (I) is shown in Scheme 2a. Here, simple pyridine derivatives such as 5-amino-2,4-dichloropyridine ((II), CAS No. 7321-93-9) are reacted in a manner familiar to the person skilled in the art with bromocarbonyl halides of the formula (IIb) in which LG represents halogen, preferably chlorine or bromine, and R⁵ and R⁶ are as defined in the general formula (I). The resulting alpha-bromocarboxamides of the formula (IIIa) are subsequently reacted with primary amines R⁷—NH₂, in which R⁷ is defined as in the general formula (I) and which are generally commercially available or known to the person skilled in the art, in a nucleophilic substitution reaction to give the intermediates of the formula V.

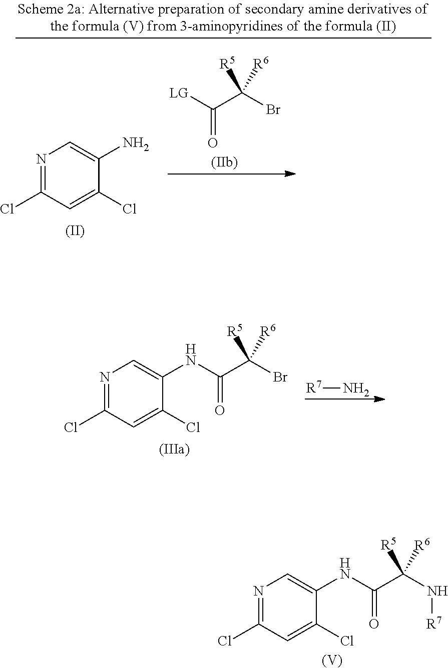

As shown in Scheme 3, the secondary amines of the formula (V) can be converted by cyclization into the dihydropyridopyrazinones of the formula (VI) (for further routes to intermediates of the formula (VI), see also US 2006/009457). To this end, compounds of the formula (V) can be reacted in the presence of a suitable base at elevated temperature (see also WO2010/96426 A2, Example 16). The subsequent alkylation to give compounds (VII) can be effected by reaction with R⁴-LG in which R⁴ is as defined in the general formula (I) and LG represents a leaving group, preferably iodide, in the presence of a suitable base such as sodium hydride, under conditions known to the person skilled in the art. Further conversion of the resulting compounds of the formula (VII) to the ester derivatives (VIII) can be performed by reaction with aminopyridines of the formula (Vita) in which A, R², R³ and n are as defined in the general formula I and in which R^{E} represents C₁-C₆-alkyl, in a palladium-catalysed coupling reaction according to Buchwald and Hartwig (see, for example, J. Organomet. Chem. (1999), 576, p 125ff). Examples of palladium sources suitable here are palladium acetate or palladium(dba) complexes, for example Pd₂(dba)₃ (CAS Nos. 51364-51-3 and 52409-22-0). The conversion depends significantly on the ligands used. The examples given in the experimental section were obtained in this way, for example through the use of (+)-BINAP (cf. also US2006/009457 A1). Some of the aminopyridines of the formula (Vila) are commercially available, or they can be prepared using methods known to the person skilled in the art.

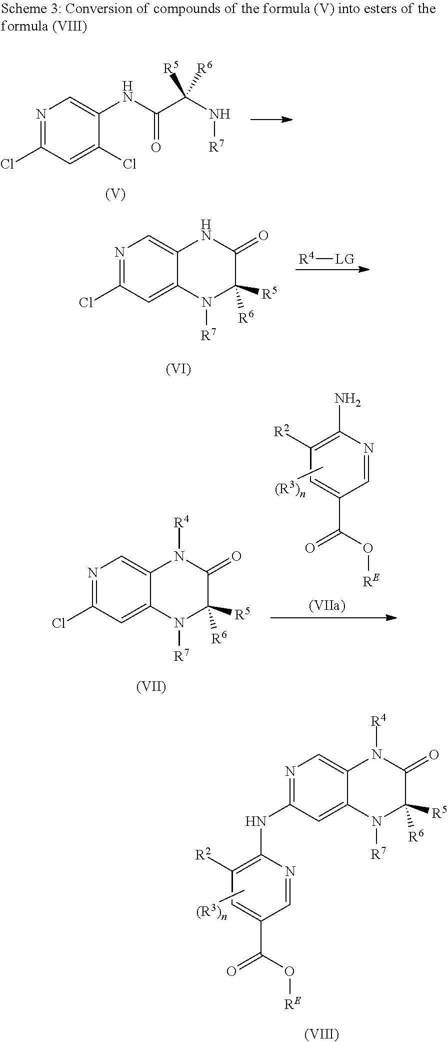

The preparation of carboxamides of the general formula (Ia) can be effected in accordance with Scheme 4 by means of hydrolysis of the respective esters of the formula (VIII) to give the corresponding carboxylic acids of the formula (IX) by methods known to the person skilled in the art. These reactions can preferably be carried out using alkali metal hydroxides such as lithium hydroxide, sodium hydroxide or potassium hydroxide in aqueous alcoholic solutions.

The carboxylic acids (IX) obtained in this manner can be converted into the carboxamides of the general formula (Ia) according to the invention by reaction with the generally commercially available amines of the formula R⁸R⁹NH, for example those shown in the working examples, in which R⁸ and R⁹ are as defined for the general formula (I), with additional activation by a method as commonly known to the person skilled in the art. Possible methods which should be mentioned here include the use of HATU, HBTU, PyBOB or T3P with the addition of a suitable base. The conversion of the carboxylic acids into their amides is described in general terms in reference books such as “Compendium of Organic Synthetic Methods”, volume I-VI (Wiley Interscience) or “The Practice of Peptide Synthesis”, Bodansky (Springer Verlag).

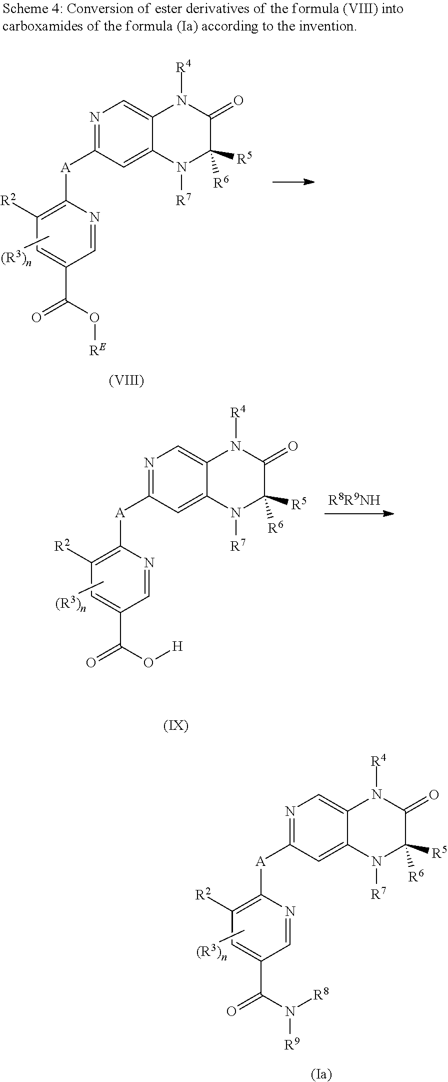

The preparation of the compounds of the formula (Ib) according to the invention having a sulphonamide group in the position of R¹ can be effected according to Scheme 5. In this context, compounds of the formula (VII) in which the chlorine may also be replaced by bromine or another leaving group, can be reacted directly, in a manner analogous to that discussed in Scheme 3 for the conversion of (VII) to (VIII), with compounds of the formula (X) in which A, R², R³, R⁸, R⁹ and n are as defined in the general formula (I) in a palladium-catalysed coupling reaction according to Buchwald and Hartwig to give the compounds of the formula (Ib) according to the invention. (see, for example, J. Med. Chem. (1996), 39, p 904ff, T. R. Jones et al.). Compounds of the formula (X) are commercially available or can be prepared by methods known to the person skilled in the art.

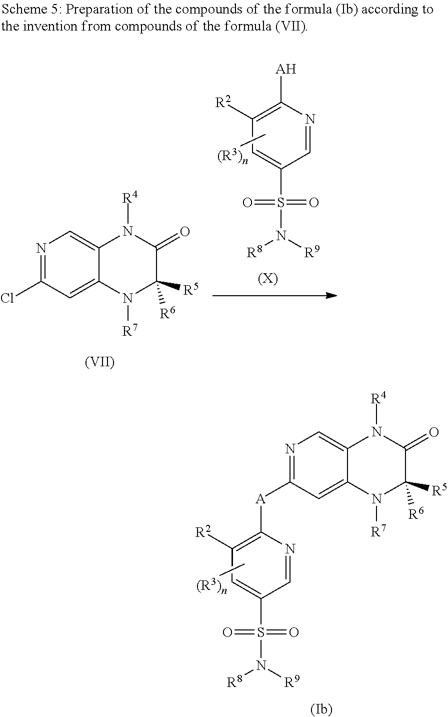

In an analogous manner, this method, as shown in Scheme 6, can also be used as an alternative method for the preparation of carboxamides of the General formula (Ia), by replacing the sulphonamide intermediates (X) with the analogous carboxamides (XI) in which A, R², R³, R⁸, and n are each as defined in the general formula (I).

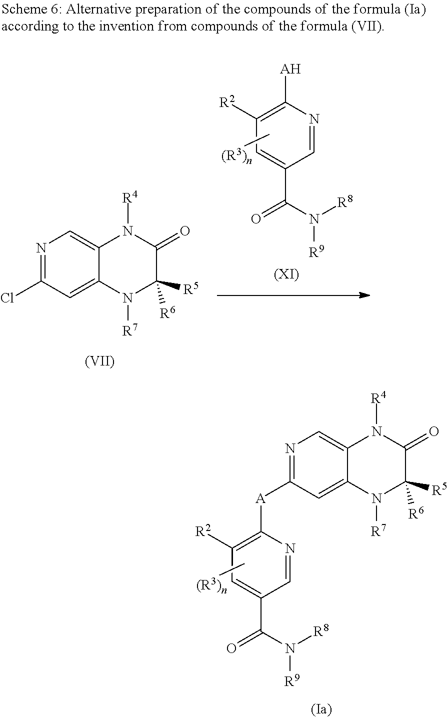

In addition, also in an analogous manner, the halogenated intermediates such as (VII), through reaction with compounds of the formula (XII) in which A, R², R³ and n are as defined in the general formula (I), and in which HetAr represents 5-membered monocyclic heteroaryl-, as defined in formula (I) for R¹, can be used to obtain compounds of the formula (Ic) according to the invention, as shown in Scheme 7:

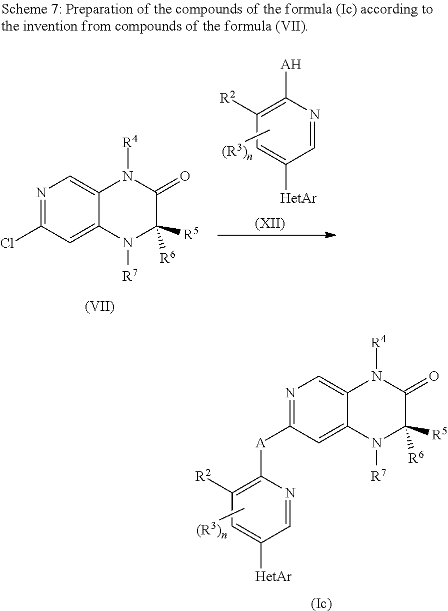

Compounds of the formula (XII) are optionally commercially available or are known to those skilled in the art. Compounds of the formula (Ic) according to the invention are additionally obtainable by, as shown in Scheme 8, reacting intermediates of the formula (XIII), which can be prepared by the methods described above and in which A, R², R³, R⁴, R⁵, R⁶, R⁷ and n are as defined in the general formula (I), and in which R^{Hal} represents a halogen, preferably bromine or iodine, in a Suzuki coupling familiar to the person skilled in the art, with a heteroaromatic boronic acid or a corresponding boronic ester in which HetAr represents 5-membered monocyclic heteroaryl-, as defined in formula (I) for R¹, and R represents hydrogen or C₁-C₄-alkyl-, —B(OR)₂ represents a pinacolyl boronate, to give the compounds of the formula (Ic) according to the invention (see also D. G. Hall, Boronic Acids, 2005 WILEY-VCH Verlag GmbH & Co. KGaA, Weinheim, ISBN 3-527-30991-8, and literature cited therein).

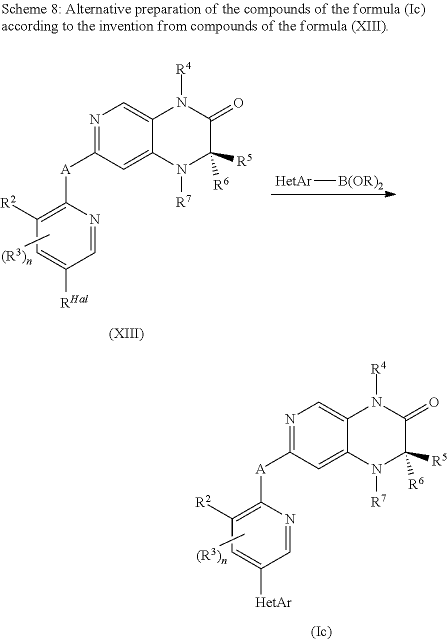

Furthermore, the compounds of the formula (Ic) according to the invention can also be formed from the ester intermediates of the formula (VIII) and carboxylic acids of the formula (IX) shown in Scheme 4, in the manner known to the person skilled in the art.

The reaction routes described allow, in the case of the use of an enantiomerically pure azidocarboxylic acid of the formula (IIa) at the start of the sequence, very substantial suppression of epimerization or racemization of the stereogenic site at the carbon atom attached to R⁵ and R⁶.

The present invention also provides the intermediates of the general formula (VIII)

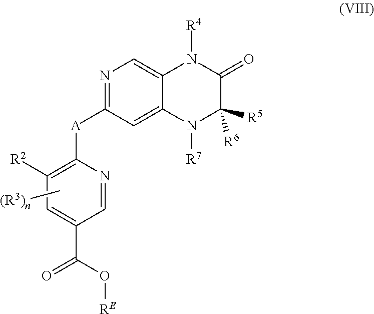

in which A, R², R³, R⁴, R⁵, R⁶, Wand n have the meanings given in the general formula (I) and R^{E} represents C₁-C₆-alkyl, which can preferably be used for preparation of the compounds of the general formula (I) according to the invention.

The present invention furthermore provides the intermediates of the general formula (IX)

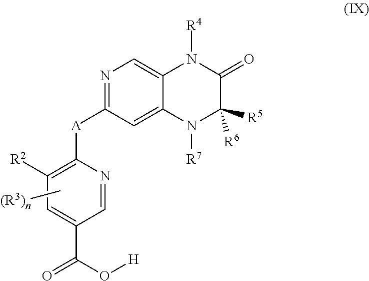

in which A, R², R³, R⁴, R⁵, R⁶, R⁷ and n have the meanings given in the general formula (I), and which can preferably be used for preparation of the compounds of the general formula (I) according to the invention.

### WORKING EXAMPLES

The examples which follow describe the preparation of the compounds according to the invention, without restricting the invention to these examples.

Firstly, there is a description of the preparation of the intermediates which are ultimately used preferentially for preparation of the compounds according to the invention.

IUPAC names were created with the aid of the nomenclature software ACD Name batch, Version 12.01, from Advanced Chemical Development, Inc., and adapted if required, for example to German-language nomenclature.

### Stoichiometry of Salt Forms

In the case of the synthesis intermediates and working examples of the invention described hereinafter, any compound specified in the form of a salt of the corresponding base or acid is generally a salt of unknown exact stoichiometric composition, as obtained by the respective preparation and/or purification process. Unless specified in more detail, additions to names and structural formulae, such as “hydrochloride”, “trifluoroacetate”, “sodium salt” or “x HCl”, “x CF₃COOH”, “x Nat” should not therefore be understood in a stoichiometric sense in the case of such salts, but have merely descriptive character with regard to the salt-forming components present therein.

This applies correspondingly if synthesis intermediates or working examples or salts thereof were obtained in the form of solvates, for example hydrates, of unknown stoichiometric composition (if they are of a defined type) by the preparation and/or purification processes described.

### Preparation of the Intermediates

### Intermediate 1.1: 7-Chloro-1-cyclopentyl-2,4-dimethyl-1,4-dihydropyrido[3,4-b]pyrazin-3(2H)-one

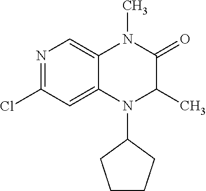 [SMILES: CC1C(=O)N(C)c2cnc(Cl)cc2N1C1CCCC1]

At 0° C., 700 mg of sodium hydride (60% in mineral oil) were added to a solution of 3 g of 7-chloro-1-cyclopentyl-2-methyl-1,4-dihydropyrido[3,4-b]pyrazin-3(2H)-one (US20060009457) in 31 ml of DMF. 1.1 ml of iodomethane were then added, and the mixture was stirred for 2.5 hours. Ice-water was added and the mixture was extracted twice with ethyl acetate. The combined organic phases were washed with saturated sodium chloride solution and dried with sodium sulphate. Chromatography on silica gel (hexane/ethyl acetate gradient) gave 2.53 g of 7-chloro-1-cyclopentyl-2,4-dimethyl-1,4-dihydropyrido[3,4-b]pyrazin-3(2H)-one.

¹H NMR (300 MHz, RT, DMSO-d6): δ=1.06 (d, 3H); 1.52-1.74 (m, 6H); 1.86-2.05 (m, 2H); 3.28 (s, 3H); 4.00 (q, 1H); 4.23 (q, 1H); 6.86 (s, 1H); 7.92 (s, 1H);

### Intermediate 1.2: 5-Hydroxy-6-nitropyridine-3-carboxylic acid

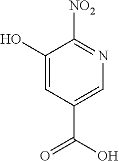 [SMILES: O=C(O)c1cnc([N+](=O)[O-])c(O)c1]

89.6 ml of nitric acid (65% strength) were added slowly to 560 ml of sulphuric acid (96% strength) such that the internal temperature never exceeded 30° C. 70 g of 5-hydroxynicotinic acid (CAS 5006-66-6) were added a little at a time at room temperature, and the mixture was stirred for 14 hours. The mixture was poured into plenty of ice-water and the resulting precipitate K1 was filtered off. The filtrate was adjusted to pH=3 using aqueous sodium hydroxide solution and extracted three times with ethyl acetate. The combined organic phases were washed with water and dried with sodium sulphate. The solvent was removed under reduced pressure. This gave a further residue K2. Both precipitates were combined to give 43 g of 5-hydroxy-6-nitropyridine-3-carboxylic acid.

¹H NMR (300 MHz, RT, DMSO-d6): δ=5.2 (bs, 1H); 8.03 (d, 1H); 8.43 (d, 1H); 12.06 (bs, 1H);

### Intermediate 1.3: Methyl 5-methoxy-6-nitropyridine-3-carboxylate

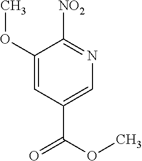

At 0° C., 847 ml of (diazomethyl)trimethylsilane were added slowly to a solution of 78 g of intermediate 1.2 in 780 ml of methanol and 780 ml of toluene, and the mixture was stirred for 14 hours. Ethyl acetate and a saturated sodium bicarbonate solution were added. The organic phase was removed and the aqueous phase was extracted twice with ethyl acetate. The combined organic phases were washed with saturated sodium chloride solution and dried over sodium sulphate. Chromatography on silica gel (hexane/ethyl acetate 8:2) gave 27 g of methyl 5-methoxy-6-nitropyridine-3-carboxylate.

¹H NMR (300 MHz, RT, DMSO-d6): δ=3.95 (s, 3H); 4.06 (s, 3H); 8.27 (d, 1H); 8.59 (d, 1H);

### Intermediate 1.4: Methyl 6-amino-5-methoxypyridine-3-carboxylate

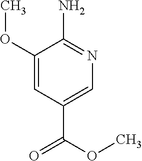

A suspension of 27 g of intermediate 1.3 and 14.2 g of iron powder in 250 ml of methanol and 250 ml of acetic acid was stirred at a bath temperature of 85° C. for 14 hours. The mixture was filtered and concentrated under reduced pressure. The residue was taken up in ethyl acetate and washed with saturated sodium bicarbonate solution. The mixture was then washed with saturated sodium chloride solution and dried over sodium sulphate. This gave 20 g of methyl 6-amino-5-methoxypyridine-3-carboxylate.

¹H NMR (300 MHz, RT, DMSO-d6): δ=3.77 (s, 3H); 3.82 (s, 3H); 6.71 (bs, 2H); 7.30 (d, 1H); 8.17 (d, 1H);

### Intermediate 1.5: Methyl 6-[(1-cyclopentyl-2,4-dimethyl-3-oxo-1,2,3,4-tetrahydropyrido[3,4-b]pyrazin-7-yl)amino]-5-methoxypyridine-3-carboxylate

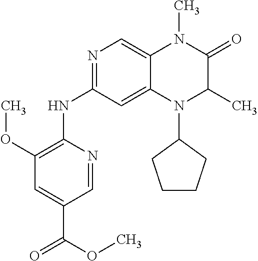 [SMILES: COC(=O)c1cnc(Nc2cc3c(cn2)N(C)C(=O)C(C)N3C2CCCC2)c(OC)c1]

445 mg of BINAP and 161 mg of palladium acetate were added to a solution of 1 g of intermediate 1.1 and 1.3 g of intermediate 1.4 in 38 ml of toluene, and the mixture was stirred for 5 minutes. 5.8 g of caesium carbonate were added and the mixture was stirred under argon at 110° C. for 2.5 hours. The mixture was diluted with ethyl acetate and twice extracted with water. The combined organic phases were washed with saturated sodium chloride solution, dried over sodium sulphate and concentrated under reduced pressure. Chromatography on silica gel (dichloromethane/methanol gradient) gave 601 mg of methyl 6-[(1-cyclopentyl-2,4-dimethyl-3-oxo-1,2,3,4-tetrahydropyrido[3,4-b]pyrazin-7-yl)amino]-5-methoxypyridine-3-carboxylate.

¹H NMR (300 MHz, RT, DMSO-d6): δ=1.08 (d, 3H); 1.57-1.89 (m, 6H); 1.97-2.14 (m, 2H); 3.29 (s, 3H); 3.85 (s, 3H); 3.92 (q, 1H); 3.98 (s, 3H); 4.20 (q, 1H); 7.56 (d, 1H); 7.86 (s, 1H); 8.10 (s, 1H); 8.12 (s, 1H); 8.39 (d, 1H);

### Intermediate 1.6: 6-[(1-Cyclopentyl-2,4-dimethyl-3-oxo-1,2,3,4-tetrahydropyrido[3,4-b]pyrazin-7-yl)amino]-5-methoxypyridine-3-carboxylic acid

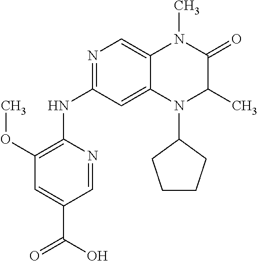 [SMILES: COc1cc(C(=O)O)cnc1Nc1cc2c(cn1)N(C)C(=O)C(C)N2C1CCCC1]

14 ml of lithium hydroxide solution (1M) were added to a solution of 601 mg of intermediate 1.5 in 28 ml of methanol and 9 ml of THF, and the mixture was stirred at room temperature for 14 hours. The mixture was adjusted to pH=5 with hydrochloric acid and extracted three times with a chloroform/methanol solution (9:1). The mixture was washed with saturated sodium chloride solution, dried over sodium sulphate and concentrated under reduced pressure. This gave 562 mg of 6-[(1-cyclopentyl-2,4-dimethyl-3-oxo-1,2,3,4-tetrahydropyrido[3,4-b]pyrazin-7-yl)amino]-5-methoxypyridinr-3-carboxylic acid.

¹H NMR (300 MHz, RT, DMSO-d6): δ=1.08 (d, 3H); 1.56-1.93 (m, 6H); 1.96-2.16 (m, 2H); 3.85-4.01 (m+s, 4H); 4.19 (q, 1H); 7.59 (d, 1H); 7.84 (s, 1H); 7.92 (s, 1H); 8.16 (s, 1H); 8.34 (d, 1H);

### Intermediate 2.1: (2R)-2-Azido-N-(4,6-dichloropyridin-3-yl)butanamide

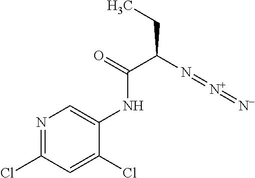 [SMILES: CC[C@@H](N=[N+]=[N-])C(=O)Nc1cnc(Cl)cc1Cl]

A solution of 4.75 g of (2R)-2-azidobutanoic acid (preparation see US20060009457) and 8.05 ml of thionyl chloride in 40 ml of dichloromethane was stirred at 50° C. for 2 hours. The mixture was concentrated completely under reduced pressure, and a solution of 3 g of 5-amino-2,4-dichloropyridine (CAS-No. 7321-93-9; preparation see US20060009457) and 6.5 ml of pyridine in 15 ml of dichloromethane were added dropwise at 0° C. The mixture was slowly warmed to room temperature and, after about 2 hours, heated to 40° C. A further 20 ml of pyridine were added and the mixture was stirred at 40° C. for 14 hours. After addition of water, the mixture was extracted twice with dichloromethane and dried over sodium sulphate. Chromatography on silica gel (hexane/ethyl acetate 80:20) gave 1.37 g of (2R)-2-azido-N-(4,6-dichloropyridin-3-yl)butanamide.

¹H NMR (300 MHz, RT, DMSO-d6): δ=1.00 (t, 3H); 1.77-1.97 (2 m, 2H); 4.09 (dd, 1H); 7.93 (s, 1H); 8.62 (s, 1H); 10.21 (s, 1H);

### Intermediate 2.2: (2R)-2-Amino-N-(4,6-dichloropyridin-3-yl)butanamide hydrochloride

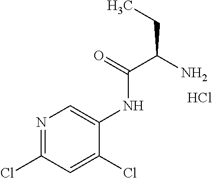

At RT and under argon, 1.2 equivalents of a trimethylphosphine solution (1M in THF) were added to a solution of 853 mg of intermediate 2.1 in 12 ml of THF. The mixture was stirred at RT for 14 hours. After addition of water, the mixture was concentrated under reduced pressure. The residue was taken up in water and extracted with dichloromethane, and the organic phase was dried over sodium sulphate. After removal of the solvent, the residue was taken up in acetone/Et₂O and the target compound was precipitated as hydrochloride using HCl (solution in diethyl ether). This gave 440 mg of (2R)-2-amino-N-(4,6-dichloropyridin-3-yl)butanamide hydrochloride.

¹H NMR (300 MHz, RT, DMSO-d6): δ=1.00 (t, 3H); 1.85-1.98 (m, 2H); 4.13 (bq, 1H); 7.95 (s, 1H); 8.48 (bs, 3H); 8.59 (s, 1H); 10.79 (s, 1H);

### Intermediate 2.3: (2R)-2-(Cyclopentylamino)-N-(4,6-dichloropyridin-3-yl)butanamide

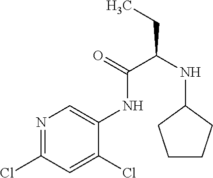 [SMILES: CC[C@@H](NC1CCCC1)C(=O)Nc1cnc(Cl)cc1Cl]

A solution of 440 mg of intermediate 2.2, 152 mg of cyclopentanone, 254 mg of sodium acetate and 946 mg of sodium triacetoxyborohydride in 20 ml of dichloromethane was stirred at room temperature for 6 hours. The mixture was added to saturated sodium bicarbonate solution and the organic phase was separated off. The aqueous phase was extracted with dichloromethane, and the combined organic phases were dried over sodium sulphate and freed of the solvent under reduced pressure. The residue was purified by chromatography on silica gel (dichloromethane/methanol 98:2). This gave 355 mg of (2R)-2-(cyclopentylamino)-N-(4,6-dichloropyridin-3-yl)butanamide.

¹H NMR (300 MHz, RT, DMSO-d6): δ=0.95 (t, 3H); 1.29-1.4 (m, 2H); 1.4-1.52 (m, 2H); 1.53-1.82 (m, 6H); 3.02 (qi, 1H); 3.14 (dd, 1H); 7.90 (s, 1H); 9.06 (s, 1H);

### Intermediate 2.4: (2R)-7-Chloro-1-cyclopentyl-2-ethyl-1,4-dihydropyrido[3,4-b]pyrazin-3(2H)-one

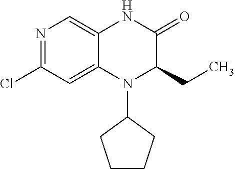 [SMILES: CC[C@@H]1C(=O)Nc2cnc(Cl)cc2N1C1CCCC1]

A solution of 355 mg of intermediate 2.3 and 1.6 ml of diisopropylethylamine in 2.2 ml of DMF was stirred at 155° C. in a closed glass tube for 96 hours. The mixture was diluted with water and extracted with dichloromethane. The solvent was removed under reduced pressure and the residue was purified by chromatography on silica gel (hexane/ethyl acetate 75:25). This gave 75 mg of (2R)-7-chloro-1-cyclopentyl-2-ethyl-1,4-dihydropyrido[3,4-b]pyrazin-3(2H)-one.

¹H NMR (300 MHz, RT, DMSO-d6): δ=0.79 (t, 3H); 1.44-1.53 (m, 1H); 1.54-1.74 (m, 7H); 1.85-1.94 (m, 1H); 1.97-2.05 (m, 1H); 3.95 (dd, 1H); 3.99-4.06 (m, 1H); 6.77 (s, 1H); 7.60 (s, 1H); 10.73 (s, 1H);

### Intermediate 2.5: (2R)-7-Chloro-1-cyclopentyl-2-ethyl-4-methyl-1,4-dihydropyrido[3,4-b]pyrazin-3(2H)-one

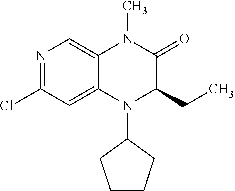 [SMILES: CC[C@@H]1C(=O)N(C)c2cnc(Cl)cc2N1C1CCCC1]

### Variant A:

At −5° C., 20 mg of sodium hydride (60% in oil) were added to a solution of 70 mg of intermediate 2.4 and 0.02 ml of iodomethane in 2 ml of DMF. After 2 hours at 0° C., water was added and the mixture was extracted 4 times with dichloromethane. The combined organic phases were dried with sodium sulphate and the solvent was removed under reduced pressure. Chromatography on silica gel (hexane/ethyl acetate 1:1) gave 57 mg of (2R)-7-chloro-1-cyclopentyl-2-ethyl-4-methyl-1,4-dihydropyrido[3,4-b]pyrazin-3(2H)-one.

### Variant B:

12 g of 7-chloro-1-cyclopentyl-2-ethyl-4-methyl-1,4-dihydropyrido[3,4-b]pyrazin-3(2H)-one (intermediate 3.4) were separated into the enantiomers by chiral HPLC (Chiralpak IC 20 μm 330×51 mm, hexane/ethanol 90:10, 250 ml/min). This gave 5.2 g of (2R)-7-chloro-1-cyclopentyl-2-ethyl-4-methyl-1,4-dihydropyrido[3,4-b]pyrazin-3(2H)-one.

¹H NMR (300 MHz, RT, DMSO-d6): δ=0.76 (t, 3H); 1.39-1.48 (m, 1H); 1.50-1.74 (m, 7H), 1.87-1.96 (m, 1H); 1.97-2.04 (m, 1H); 3.29 (s, 3H); 4.01-4.09 (m, 2H); 6.84 (s, 1H); 7.88 (s, 1H);

### Intermediate 2.6: Ethyl 5-bromo-6-{[(dimethylamino)methylidene]amino}pyridine-3-carboxylate

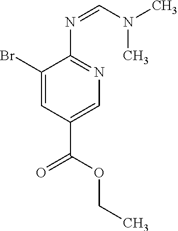 [SMILES: CCOC(=O)c1cnc(/N=C\N(C)C)c(Br)c1]

10 g of ethyl 6-amino-5-bromonicotinate (CAS 850429-51-5) in 53 ml of 1,1-dimethoxy-N,N-dimethylmethanamine were stirred at room temperature for 5 hours. The solution was concentrated under reduced pressure and the residue was crystallized from methanol. This gave 10.6 g of ethyl 5-bromo-6-{[(dimethylamino)methylidene]amino}pyridine-3-carboxylate.

¹H NMR (300 MHz, RT, DMSO-d6): δ=1.30 (t, 3H); 3.10 (s, 3H); 3.17 (s, 3H); 4.28 (q, 2H); 8.22 (d, 1H); 8.61 (s, 1H); 8.64 (d, 1H);

### Intermediate 2.7: Ethyl 6-{[(dimethylamino)methylidene]amino}-5-ethenylpyridine-3-carboxylate

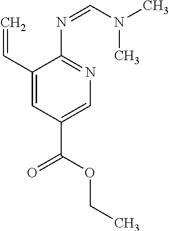 [SMILES: C=Cc1cc(C(=O)OCC)cnc1/N=C\N(C)C]

A solution of 10.6 g of intermediate 2.6, 926 mg of triphenylphosphine, 2.479 g of palladiumdichlorobis(triphenylphosphine), 14.2 g of potassium ethenyltrifluoroborate and 40.3 g of caesium carbonate in 109 ml of THF and 10.9 ml of water was heated at 85° C. for 3.5 hours. The mixture was diluted with ethyl acetate and extracted with semisaturated sodium bicarbonate solution. The organic phase was dried with sodium sulphate and concentrated under reduced pressure. The residue was purified by chromatography on silica gel (hexane/ethyl acetate gradient). This gave 4.05 g of ethyl 6-{[(dimethylamino)methylidene]amino}-5-ethenylpyridine-3-carboxylate.

¹H NMR (300 MHz, RT, DMSO-d6): δ=1.32 (t, 3H); 3.07 (s, 3H); 3.16 (s, 3H); 4.30 (q, 2H); 5.34 (dd, 1H); 5.92 (dd, 1H); 7.20 (dd, 1H); 8.18 (d, 1H); 8.61-8.65 (m, 2H);

### Intermediate 2.8: Ethyl 6-amino-5-ethenylpyridine-3-carboxylate

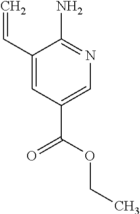

A solution of 2.5 g of intermediate 2.7, 44.5 ml of concentrated hydrochloric acid, 31.2 ml of ethanol and 21.7 ml of water was stirred at room temperature for 72 hours. The mixture was adjusted to pH=7 using aqueous sodium hydroxide solution and extracted with ethyl acetate. The organic phase was dried over sodium sulphate and concentrated under reduced pressure. The residue was purified by chromatography on silica gel (hexane/ethyl acetate gradient). This gave 600 mg of ethyl 6-amino-5-ethenylpyridine-3-carboxylate. This reaction was carried out a second time analogously using 1.5 g of intermediate 2.7, giving 400 mg of ethyl 6-amino-5-ethenylpyridine-3-carboxylate.

¹H NMR (300 MHz, RT, DMSO-d6): δ=1.29 (t, 3H); 4.24 (q, 2H); 5.32 (dd, 1H); 5.74 (dd, 1H); 6.82 (dd, 1H); 6.89 (s, 2H); 7.97 (d, 1H); 8.46 (d, 1H);

### Intermediate 2.9: Ethyl 6-amino-5-ethylpyridine-3-carboxylate

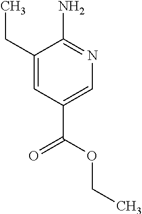

587 mg of palladium on carbon (10%) were added to a solution of 1.0 g of ethyl 6-amino-5-ethenylpyridine-3-carboxylate (prepared as described under intermediate 2.8) in 161 ml of ethanol, and the mixture was stirred under an atmosphere of hydrogen at room temperature for 2 hours. The catalyst was then filtered off and the mixture was concentrated under reduced pressure. The residue was purified by chromatography on silica gel (hexane/ethyl acetate gradient). This gave 984 mg of ethyl 6-amino-5-ethylpyridine-3-carboxylate.

¹H NMR (300 MHz, RT, DMSO-d6): δ=1.13 (t, 3H); 1.28 (t, 3H); 2.41 (q, 2H); 4.22 (q, 2H); 6.65 (s, 2H); 7.65 (d, 1H); 8.39 (d, 1H);

### Intermediate 2.10: Ethyl 6-{[(2R)-1-cyclopentyl-2-ethyl-4-methyl-3-oxo-1,2,3,4-tetrahydropyrido[3,4-b]pyrazin-7-yl]amino}-5-ethylpyridine-3-carboxylate

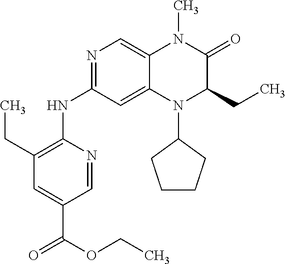 [SMILES: CCOC(=O)c1cnc(Nc2cc3c(cn2)N(C)C(=O)[C@@H](CC)N3C2CCCC2)c(CC)c1]

Under an atmosphere of argon, a solution of 700 mg of intermediate 2.5, 107 mg of palladium(II) acetate, 297 mg of (R)-(+)-2,2′-bis(diphenylphospino)-1,1′-binaphthyl, 3.88 g of caesium carbonate and 925 mg of intermediate 2.9 in 25 ml of toluene was stirred at 120° C. for 3 hours. The mixture was diluted with ethyl acetate, the precipitate was filtered off and the organic phase was washed with saturated sodium chloride solution. The organic phase was dried over sodium sulphate and concentrated under reduced pressure. The residue was purified by chromatography on silica gel (ethyl acetate). This gave 300 mg of ethyl 6-{[(2R)-1-cyclopentyl-2-ethyl-4-methyl-3-oxo-1,2,3,4-tetrahydropyrido[3,4-b]pyrazin-7-yl]amino}-5-ethylpyridine-3-carboxylate.

¹H NMR (300 MHz, RT, DMSO-d6): δ=0.78 (t, 3H); 1.20 (t, 3H); 1.31 (t, 3H); 1.41-1.53 (m, 1H); 1.54-1.89 (2 m, 7H); 1.92-2.13 (m, 2H); 2.75 (q, 2H); 3.31 (s, 3H); 3.87-4.00 (m, 1H); 4.03 (dd, 1H); 4.30 (q, 2H); 7.81 (s, 1H); 7.84 (s, 1H); 7.89 (d, 1H); 8.40 (s, 1H); 8.60 (d, 1H);

### Intermediate 2.11: 6-{[(2R)-1-Cyclopentyl-2-ethyl-4-methyl-3-oxo-1,2,3,4-tetrahydropyrido[3,4-b]pyrazin-7-yl]amino}-5-ethylpyridine-3-carboxylic acid

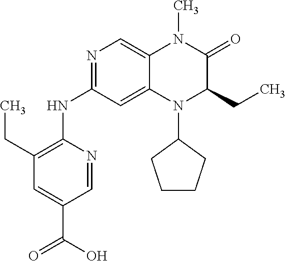 [SMILES: CCc1cc(C(=O)O)cnc1Nc1cc2c(cn1)N(C)C(=O)[C@@H](CC)N2C1CCCC1]

A solution of 298 mg of intermediate 2.10 in 13.6 ml of methanol, 4.2 ml of THF and 6.6 ml of lithium hydroxide solution (1M) was stirred at room temperature for 2 hours. The pH was adjusted to pH=5 using hydrochloric acid (1M), and the mixture was extracted with chloroform/methanol (9:1). The organic phase was dried over sodium sulphate and concentrated completely under reduced pressure. This gave 274 mg of 6-{[(2R)-1-cyclopentyl-2-ethyl-4-methyl-3-oxo-1,2,3,4-tetrahydropyrido[3,4-b]pyrazin-7-yl]amino}-5-ethylpyridin-3-carboxylic acid.

¹H NMR (300 MHz, RT, DMSO-d6): δ=0.77 (t, 3H); 1.20 (t, 3H); 1.40-1.56 (m, 1H); 1.56-1.91 (2 m, 7H); 1.93-2.13 (m, 2H); 2.73 (q, 2H); 3.31 (s, 3H); 3.86-3.98 (m, 1H); 4.03 (dd, 1H); 7.81-7.86 (m, 2H); 7.89 (d, 1H); 8.33 (bs, 1H); 8.58 (d, 1H); 12.81 (bs, 1H);

### Intermediate 3.1: 2-Bromo-N-(4,6-dichloro-3-pyridinyl)butanamide

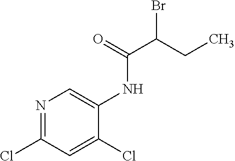

At 0° C., 260 ml of 2-bromobutanoyl bromide were slowly added dropwise to a suspension of 194 g of 5-amino-2,4-dichloropyridine (CAS-No. 7321-93-9) and 388 g of potassium carbonate in 3.881 of diethyl ether. The mixture was filtered and the filter cake was washed with diethyl ether. The filter cake was dissolved in dichloromethane and the resulting solution was washed with water and saturated sodium chloride solution. The organic phase was dried over sodium sulphate and concentrated under reduced pressure. The residue was stirred with hexane, once more filtered off with suction and dried under reduced pressure. This gave 150 g of 2-bromo-N-(4,6-dichloro-3-pyridinyl)butanamide.

¹H NMR (300 MHz, RT, DMSO-d6): δ=0.98 (t, 3H); 1.88-2.01 (m, 1H); 2.03-2.16 (m, 1H); 4.68 (t, 1H); 7.92 (s, 1H); 8.63 (s, 1H); 10.31 (s, 1H);

### Intermediate 3.2: 2-(Cyclopentylamino)-N-(4,6-dichloro-3-pyridinyl)butanamide

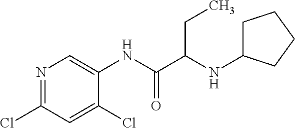 [SMILES: CCC(NC1CCCC1)C(=O)Nc1cnc(Cl)cc1Cl]

A solution of 130 g of intermediate 3.1, 119 ml of N,N-diisopropylethylamine and 37.4 ml of cyclopentylamine in 1.3 l of toluene was stirred at a bath temperature of 150° C. for 24 hours. The mixture was filtered, the solid was washed with ethyl acetate and the combined filtrates were concentrated completely under reduced pressure. This gave 138 g of 2-(cyclopentylamino)-N-(4,6-dichloro-3-pyridinyl)butanamide which still contained some toluene.

¹H NMR (300 MHz, RT, DMSO-d6): δ=0.95 (t, 3H); 12.9-1.83 (3 m, 10H); 3.02 (qi, 1H); 3.15 (t, 1H); 7.90 (s, 1H); 9.05 (s, 1H);

### Intermediate 3.3: 7-Chloro-1-cyclopentyl-2-ethyl-1,4-dihydropyrido[3,4-b]pyrazin-3(2H)-one

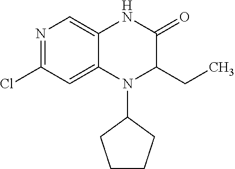 [SMILES: CCC1C(=O)Nc2cnc(Cl)cc2N1C1CCCC1]

A solution of 32.5 g of intermediate 3.2 and 53.6 ml of N,N-diisopropylethylamine in 195 ml of 1,3-dimethylimidazolidin-2-one was stirred at a bath temperature of 210° C., and during this time the N,N-diisopropylethylamine was distilled off slowly. The mixture was stirred for a further 5 hours at a bath temperature of 220° C. After cooling, the mixture was taken up in water and extracted three times with ethyl acetate. The combined organic phases were washed with water, dried over sodium sulphate and concentrated under reduced pressure. Chromatography on silica gel (hexane/ethyl acetate 8:2) gave 20.3 g of 7-chloro-1-cyclopentyl-2-ethyl-1,4-dihydropyrido[3,4-b]pyrazin-3(2H)-one.

¹H NMR (300 MHz, RT, DMSO-d6): δ=0.79 (t, 3H); 1.44-1.53 (m, 1H); 1.54-1.74 (m, 7H); 1.85-1.94 (m, 1H); 1.97-2.05 (m, 1H); 3.95 (dd, 1H); 3.99-4.06 (m, 1H); 6.77 (s, 1H); 7.60 (s, 1H); 10.73 (s, 1H);

### Intermediate 3.4: 7-Chloro-1-cyclopentyl-2-ethyl-1,4-dihydro-4-methylpyrido[3,4-b]pyrazin-3(2H)-one

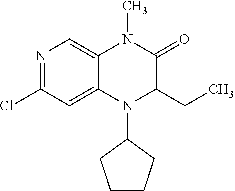 [SMILES: CCC1C(=O)N(C)c2cnc(Cl)cc2N1C1CCCC1]

At −5° C., 2.69 g of sodium hydride (60% in oil) were added to a solution of 20.3 g of intermediate 3.3 and 7 ml of iodomethane in 203 ml of DMF. After 0.5 hour at 0° C., water was added and the mixture was extracted three times with ethyl acetate. The combined organic phases were dried with sodium sulphate and the solvent was removed under reduced pressure. Chromatography on silica gel (hexane/ethyl acetate 8:2) gave 18.1 g of 7-chloro-1-cyclopentyl-2-ethyl-4-methyl-1,4-dihydropyrido[3,4-b]pyrazin-3(2H)-one.

¹H NMR (300 MHz, RT, DMSO-d6): δ=0.76 (t, 3H); 1.39-1.48 (m, 1H); 1.50-1.74 (m, 7H), 1.87-1.96 (m, 1H); 1.97-2.04 (m, 1H); 3.29 (s, 3H); 4.01-4.09 (m, 2H); 6.84 (s, 1H); 7.88 (s, 1H);

### Intermediate 3.5: Methyl 6-[(1-cyclopentyl-2-ethyl-1,2,3,4-tetrahydro-4-methyl-3-oxopyrido[3,4-b]pyrazin-7-yl)amino]-5-methoxypyridin-3-carboxylate

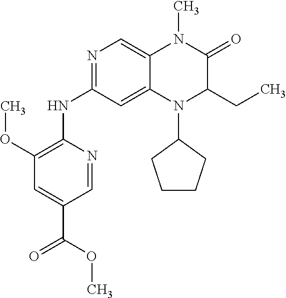 [SMILES: CCC1C(=O)N(C)c2cnc(Nc3ncc(C(=O)OC)cc3OC)cc2N1C1CCCC1]

Under an atmosphere of argon, a solution of 1 g of intermediate 3.4, 1.24 g of intermediate 1.4, 626 mg of palladium(II) acetate, 895 mg of (R)-(+)-2,2′-bis(diphenylphospino)-1,1′-binaphthyl and 658 mg of sodium tert-butoxide in 60 ml of dioxane was heated in a microwave oven at 110° C. for 1 hour. Saturated sodium bicarbonate solution was added and the mixture was extracted 3 times with ethyl acetate. The organic phase was dried over sodium sulphate and concentrated under reduced pressure. The residue was purified by chromatography on silica gel (hexane/ethyl acetate gradient). This gave 210 mg of methyl 6-[(1-cyclopentyl-2-ethyl-1,2,3,4-tetrahydro-4-methyl-3-oxopyrido[3,4-b]pyrazin-7-yl)amino]-5-methoxypyridin-3-carboxylate.

¹H NMR (300 MHz, RT, DMSO-d6): δ=0.76 (t, 3H); 1.43-1.55 (m, 1H); 1.56-1.93 (2 m, 7H); 1.96-2.12 (m, 2H); 3.30 (s, 3H); 3.85 (s, 3H); 3.91-4.00 (m+s, 1+3H); 4.06 (dd, 1H); 7.56 (d, 1H); 7.82 (s, 1H); 8.07 (s, 1H); 8.10 (s, 1H); 8.39 (d, 1H);

### Intermediate 3.6: 6-[(1-Cyclopentyl-2-ethyl-4-methyl-3-oxo-1,2,3,4-tetrahydropyrido[3,4-b]pyrazin-7-yl)amino]-5-ethylpyridine-3-carboxylate

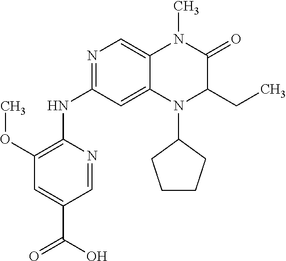 [SMILES: CCC1C(=O)N(C)c2cnc(Nc3ncc(C(=O)O)cc3OC)cc2N1C1CCCC1]

0.02 ml of aqueous sodium hydroxide solution (5N) was added to a solution of 92 mg of intermediate 3.5 in 4.6 ml of methanol and 1.2 ml of water, and the mixture was stirred at room temperature for 14 hours. 0.01 ml of aqueous sodium hydroxide solution (5N) was then added and the mixture was stirred at 55° C. for 2 hours. The mixture was adjusted to pH=5-6 with hydrochloric acid and concentrated significantly under reduced pressure. The residue was taken up in methanol/chloroform 1:8 and the aqueous phase was separated off. The organic phase was dried over sodium sulphate and concentrated completely under reduced pressure. The residue was purified by chromatography on silica gel (dichloromethane/methanol 8:2). This gave 55 mg of 6-[(1-cyclopentyl-2-ethyl-4-methyl-3-oxo-1,2,3,4-tetrahydropyrido[3,4-b]pyrazin-7-yl)amino]-5-ethylpyridine-3-carboxylate.

¹H NMR (300 MHz, RT, DMSO-d6): δ=0.75 (t, 1H); 1.40-1.55 (m, 1H); 1.55-1.75 (m, 3H); 1.75-2.15 (2 m, 6H); 3.29 (s, 1H); 3.86-4.00 (m+s, 1+3H); 4.04 (dd, 1H); 7.61 (bs, 1H); 7.79 (bs, 1H); 7.87 (bs, 1H); 8.12 (bs, 1H); 8.36 (bs, 1H);

### Intermediate 4.1: Methyl 6-[[(2R)-1-cyclopentyl-2-ethyl-1,2,3,4-tetrahydro-4-methyl-3-oxopyrido[3,4-b]pyrazin-7-yl]amino]-5-methoxypyridine-3-carboxylate

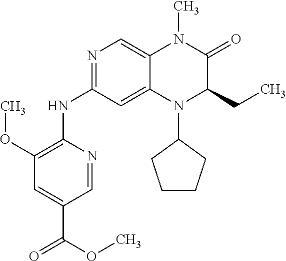 [SMILES: CC[C@@H]1C(=O)N(C)c2cnc(Nc3ncc(C(=O)OC)cc3OC)cc2N1C1CCCC1]

In analogy to the preparation of intermediate 3.5, intermediate 4.1 was prepared from 5.17 g of intermediate 2.5 and 6.41 g of intermediate 1.4. This gave 3.2 g of methyl 6-[[(2R)-1-cyclopentyl-2-ethyl-1,2,3,4-tetrahydro-4-methyl-3-oxopyrido[3,4-b]pyrazin-7-yl]amino]-5-methoxypyridine-3-carboxylate.

¹H NMR (300 MHz, RT, DMSO-d6): δ=0.76 (t, 3H); 1.42-1.55 (m, 1H); 1.55-1.93 (2 m, 7H); 1.95-2.14 (m, 2H); 3.30 (s, 3H); 3.85 (s, 3H); 3.91-4.00 (m+s, 1+3H); 4.06 (dd, 1H); 7.56 (d, 1H); 7.83 (s, 1H); 8.07 (s, 1H); 8.10 (s, 1H); 8.39 (d, 1H);

### Intermediate 4.2: 6-[[(2R)-1-Cyclopentyl-2-ethyl-1,2,3,4-tetrahydro-4-methyl-3-oxopyrido[3,4-b]pyrazin-7-yl]amino]-5-methoxypyridine-3-carboxylic acid

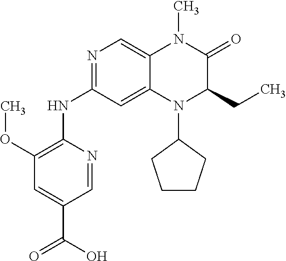 [SMILES: CC[C@@H]1C(=O)N(C)c2cnc(Nc3ncc(C(=O)O)cc3OC)cc2N1C1CCCC1]

In analogy to the preparation of intermediate 3.6, intermediate 4.2 was prepared from 2.3 g of intermediate 4.1. This gave 2.2 g of 6-[[(2R)-1-cyclopentyl-2-ethyl-1,2,3,4-tetrahydro-4-methyl-3-oxopyrido[3,4-b]pyrazin-7-yl]amino]-5-methoxypyridine-3-carboxylic acid.

¹H NMR (300 MHz, RT, DMSO-d6): δ=0.76 (t, 3H); 1.41-1.55 (m, 1H); 1.56-1.75 (m, 3H; 1.75-1.94 (m, 4H); 1.96-2.13 (m, 2H); 3.30 (s, 3H); 3.89-4.02 (m+s, 1+3H); 4.06 (dd, 1H); 7.56 (d, 1H); 7.82 (s, 1H); 8.05 (bs, 1H); 8.08 (s, 1H); 8.37 (d, 1H);

### Intermediate 5.1: Methyl 6-amino-5-methylpyridine-3-carboxylate

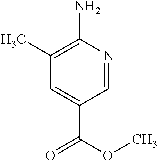

A solution of 2 g of 6-amino-5-methylpyridine-3-carbonitrile (CAS 183428-91-3) in 40 ml of methanol and 18 ml of concentrated sulphuric acid was heated at reflux for 3 hours. The mixture was added to ice-water, made basic with sodium hydroxide and extracted with ethyl acetate. The organic phase was washed with saturated sodium chloride solution, dried over sodium sulphate and concentrated under reduced pressure. This gave 1.95 g of methyl 6-amino-5-methylpyridine-3-carboxylate.

¹H NMR (300 MHz, RT, DMSO-d6): δ=2.06 (s, 3H); 3.75 (s, 3H); 6.63 (bs, 2H); 7.68 (d, 1H); 8.39 (d, 1H);

### Intermediate 5.2: Methyl 6-[[(2R)-1-cyclopentyl-2-ethyl-1,2,3,4-tetrahydro-4-methyl-3-oxopyrido[3,4-b]pyrazin-7-yl]amino]-5-methylpyridine-3-carboxylate

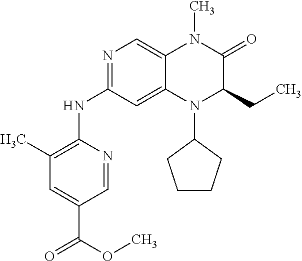 [SMILES: CC[C@@H]1C(=O)N(C)c2cnc(Nc3ncc(C(=O)OC)cc3C)cc2N1C1CCCC1]

Analogously to the preparation of intermediate 3.5, intermediate 5.2 was prepared from 700 mg of intermediate 2.5 and 791 mg of intermediate 5.1. This gave 395 mg of methyl 6-[[(2R)-1-cyclopentyl-2-ethyl-1,2,3,4-tetrahydro-4-methyl-3-oxopyrido[3,4-b]pyrazin-7-yl]amino]-5-methylpyridine-3-carboxylate.

¹H NMR (300 MHz, RT, DMSO-d6): δ=0.76 (t, 3H); 1.39-1.55 (m, 1H); 1.55-1.90 (2 m, 7H); 1.92-2.13 (m, 2H); 2.32 (s, 3H); 3.30 (s, 3H); 3.82 (s, 3H); 3.82-3.97 (m, 1H); 4.03 (dd, 1H); 7.82 (s, 1H); 7.83 s, 1H); 7.93 (d, 1H); 8.39 (s, 1H), 8.59 (d, 1H);

### Intermediate 5.3: 6-[[(2R)-1-Cyclopentyl-2-ethyl-1,2,3,4-tetrahydro-4-methyl-3-oxopyrido[3,4-b]pyrazin-7-yl]amino]-5-methylpyridine-3-carboxylic acid

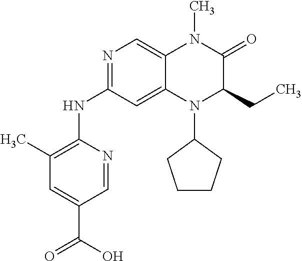 [SMILES: CC[C@@H]1C(=O)N(C)c2cnc(Nc3ncc(C(=O)O)cc3C)cc2N1C1CCCC1]

In analogy to the preparation of intermediate 3.6, intermediate 5.3 was prepared from 385 mg of intermediate 5.2. This gave 226 mg of 6-[[(2R)-1-cyclopentyl-2-ethyl-1,2,3,4-tetrahydro-4-methyl-3-oxopyrido[3,4-b]pyrazin-7-yl]amino]-5-methylpyridine-3-carboxylic acid.

¹H NMR (300 MHz, RT, DMSO-d6): δ=0.77 (t, 1H); 1.45-1.88 (3 m, 8H); 1.99-3.13 (m, 2H); 2.36 (s, 3H); 3.31 (s, 3H); 3.95 (qi, 1H); 4.13 (dd, 1H); 7.73 (s, 1H); 7.83 (s, 1H); 7.98 (d, 1H); 8.60 (d, 1H); 8.87 (bs, 1H);

### Preparation of the Compounds According to the Invention

### Example 1

### 6-{[1-Cyclopentyl-2,4-dimethyl-3-oxo-1,2,3,4-tetrahydropyrido[3,4-b]pyrazin-7-yl]amino}-N-cyclopropyl-5-methoxypyridine-3-carboxamide

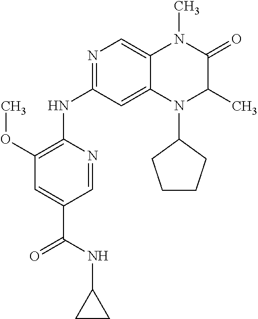 [SMILES: COc1cc(C(=O)NC2CC2)cnc1Nc1cc2c(cn1)N(C)C(=O)C(C)N2C1CCCC1]

A solution of 200 mg of intermediate 1.6, 390 mg of TBTU, 335 mg of potassium carbonate and 0.08 ml of cyclopropylamine in 10 ml of DMF was stirred at room temperature for 14 hours. The mixture was diluted with ethyl acetate and washed in each case twice with saturated sodium bicarbonate solution and saturated sodium chloride solution, dried over sodium sulphate and concentrated under reduced pressure. The residue was purified by chromatography (silica gel, ethyl acetate/methanol gradient). This gave 175 mg of 6-{[1-cyclopentyl-2,4-dimethyl-3-oxo-1,2,3,4-tetrahydropyrido[3,4-b]pyrazin-7-yl]amino}-N-cyclopropyl-5-methoxypyridine-3-carboxamide.

¹H NMR (400 MHz, RT, DMSO-d6): δ=0.53-0.61 (m, 2H); 0.67-0.75 (m, 2H); 1.08 (d, 3H); 1.58-1.80 (m, 6H); 1.99-2.15 (m, 2H); 2.77-2.88 (m, 1H); 3.29 (s, 3H); 3.68-3.99 m+s, 4H); 4.19 (q, 1H); 7.61 (d, 1H); 7.84 (s, 1H); 7.92 (s, 1H); 8.12 (s, 1H); 8.29 (d, 1H); 8.42 (d, 1H);

### Example 2

### 6-{[(2R)-1-Cyclopentyl-2,4-dimethyl-3-oxo-1,2,3,4-tetrahydropyrido[3,4-b]pyrazin-7-yl]amino}-N-cyclopropyl-5-methoxypyridine-3-carboxamide

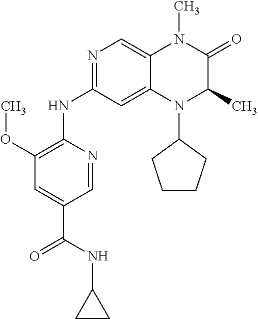 [SMILES: COc1cc(C(=O)NC2CC2)cnc1Nc1cc2c(cn1)N(C)C(=O)[C@@H](C)N2C1CCCC1]

238 mg of 6-{[1-cyclopentyl-2,4-dimethyl-3-oxo-1,2,3,4-tetrahydropyrido[3,4-b]pyrazin-7-yl]amino}-N-cyclopropyl-5-methoxypyridine-3-carboxamide were separated into the enantiomers by chiral HPLC (Chiralcel OD-H 5 μm 250×30 mm, hexane/ethanol 90:10+0.1% diethylamine (v/v), 25 ml/min). This gave 49 mg of 6-{[(2R)-1-cyclopentyl-2,4-dimethyl-3-oxo-1,2,3,4-tetrahydropyrido[3,4-b]pyrazin-7-yl]amino}-N-cyclopropyl-5-methoxypyridine-3-carboxamide.

Optical rotation: [α_{D}=−125.7°+/−0.09° (c=4.2, DMSO)].

### Example 3

### 6-{[(2R)-1-Cyclopentyl-2-ethyl-4-methyl-3-oxo-1,2,3,4-tetrahydropyrido[3,4-b]pyrazin-7-yl]amino}-N-cyclopropyl-5-ethylpyridine-3-carboxamide

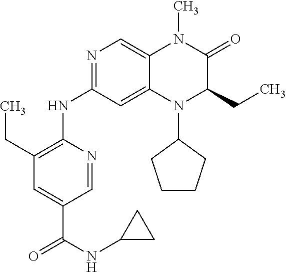 [SMILES: CCc1cc(C(=O)NC2CC2)cnc1Nc1cc2c(cn1)N(C)C(=O)[C@@H](CC)N2C1CCCC1]

A solution of 80 mg of intermediate 2.11, 121 mg of TBTU, 130 mg of potassium carbonate and 0.32 mg of cyclopropylamine in 6.4 ml of DMF was stirred at room temperature for 14 hours. The mixture was diluted with ethyl acetate and washed in each case twice with saturated sodium bicarbonate solution and saturated sodium chloride solution, dried over sodium sulphate and concentrated under reduced pressure. The residue was purified by chromatography (silica gel, ethyl acetate/methanol gradient). This gave 62 mg of 6-{[(2R)-1-cyclopentyl-2-ethyl-4-methyl-3-oxo-1,2,3,4-tetrahydropyrido[3,4-b]pyrazin-7-yl]amino}-N-cyclopropyl-5-ethylpyridin-3-carboxamide.

¹H NMR (300 MHz, RT, DMSO-d6): δ=0.52-0.6 (m, 2H); 0.65-0.73 (m, 2H); 0.78 (t, 3H); 1.21 (t, 3H); 1.40-1.55 (m, 1H); 1.55-1.89 (2 m, 7H); 1.93-2.13 (m, 2H); 2.66-2.76 (m, 2H); 2.81 (dq, 1H); 3.30 (s, 1H); 3.85-3.97 (m, 1H); 3.99-4.06 (m, 1H); 7.81 (s, 1H); 7.83 (s, 1H); 1.87 (d, 1H); 8.17 (s, 1H); 8.38 (d, 1H); 8.51 (d, 1H);

### Example 4

### 6-[(1-Cyclopentyl-2-ethyl-1,2,3,4-tetrahydro-4-methyl-3-oxopyrido[3,4-b]pyrazin-7-yl)amino]-N-cyclopropyl-5-methoxy-3-pyridinecarboxamide

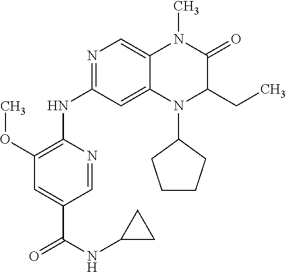 [SMILES: CCC1C(=O)N(C)c2cnc(Nc3ncc(C(=O)NC4CC4)cc3OC)cc2N1C1CCCC1]

Analogously to Example 3, 50 mg of intermediate 3.6 and 20 mg of cyclopropylamine gave 42 mg of 6-[(1-cyclopentyl-2-ethyl-1,2,3,4-tetrahydro-4-methyl-3-oxopyrido[3,4-b]pyrazin-7-yl)amino]-N-cyclopropyl-5-methoxy-3-pyridinecarboxamide.

¹H NMR (300 MHz, RT, DMSO-d6): δ=0.52-0.6 (m, 2H); 0.66-0.82 (m, 5H); 1.39-1.56 (m, 1H); 1.56-1.93 (2 m, 7H); 1.95-2.14 (m, 2H); 2.77-2.88 (m, 1H); 3.30 (s, 3H); 3.87-4.00 (m+s, 1+3H); 4.05 (dd, 1H); 7.61 (s, 1H); 7.81 (s, 1H); 7.90 (s, 1H); 8.09 (s, 1H); 8.29 (s, 1H); 8.43 (d, 1H);

### Example 5

### 6-[[(2R)-1-Cyclopentyl-2-ethyl-1,2,3,4-tetrahydro-4-methyl-3-oxopyrido[3,4-b]pyrazin-7-yl)amino]-N-cyclopropyl-5-methoxy-3-pyridinecarboxamide

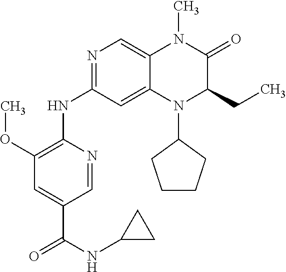 [SMILES: CC[C@@H]1C(=O)N(C)c2cnc(Nc3ncc(C(=O)NC4CC4)cc3OC)cc2N1C1CCCC1]

Analogously to Example 3, 150 mg of intermediate 4.2 and 49 mg of cyclopropylamine gave 92 mg of 6-[[(2R)-1-cyclopentyl-2-ethyl-1,2,3,4-tetrahydro-4-methyl-3-oxopyrido[3,4-b]pyrazin-7-yl)amino]-N-cyclopropyl-5-methoxy-3-pyridinecarboxamide.

¹H NMR (300 MHz, RT, DMSO-d6): δ=0.52-0.6 (m, 2H); 0.66-0.82 (m, 5H); 1.39-1.56 (m, 1H); 1.56-1.93 (2 m, 7H); 1.95-2.14 (m, 2H); 2.77-2.88 (m, 1H); 3.30 (s, 3H); 3.87-4.00 (m+s, 1+3H); 4.05 (dd, 1H); 7.61 (s, 1H); 7.81 (s, 1H); 7.90 (s, 1H); 8.09 (s, 1H); 8.29 (s, 1H); 8.43 (d, 1H);

### Example 6

### 6-[(1-Cyclopentyl-2,4-dimethyl-3-oxo-1,2,3,4-tetrahydropyrido[3,4-b]pyrazin-7-yl)amino]-5-methoxy-N-(1-methylpiperidin-4-yl)pyridine-3-carboxamide

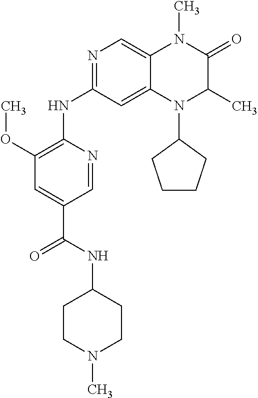 [SMILES: COc1cc(C(=O)NC2CCN(C)CC2)cnc1Nc1cc2c(cn1)N(C)C(=O)C(C)N2C1CCCC1]

Analogously to Example 3, 203 mg of intermediate 1.6 and 148 mg of 4-amino-1-methylpiperidine gave 87 mg of 6-[(1-cyclopentyl-2,4-dimethyl-3-oxo-1,2,3,4-tetrahydropyrido[3,4-b]pyrazin-7-yl)amino]-5-methoxy-N-(1-methylpiperidin-4-yl)pyridine-3-carboxamide.

¹H NMR (300 MHz, RT, DMSO-d6): δ=1.08 (d, 3H); 1.5-2.15 (m, 12H); 2.73-2.84 (m, 2H); 3.29 (s, 3H), 3.64-3.81 (m, 1H); 3.86-4.01 (m+s, 1+3H); 4.19 (q, 1H); 7.63 (d, 1H); 7.85 (s, 1H); 7.92 (s, 1H); 8.13 (s, 1H); 8.20 (d, 1H); 8.32 (d, 1H);

### Example 7

### 6-[[(2R)-1-Cyclopentyl-2-ethyl-1,2,3,4-tetrahydro-4-methyl-3-oxopyrido[3,4-b]pyrazin-7-yl]amino]-5-methoxy-N-(2,2,2-trifluoroethyl)-3-pyridinecarboxamide

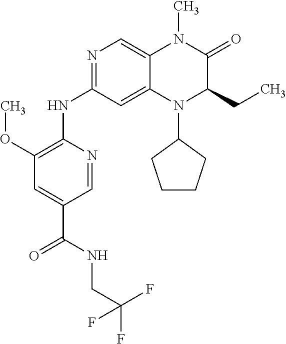 [SMILES: CC[C@@H]1C(=O)N(C)c2cnc(Nc3ncc(C(=O)NCC(F)(F)F)cc3OC)cc2N1C1CCCC1]

Analogously to Example 3, 50 mg of intermediate 4.2 and 35 mg of 2,2,2-trifluoroethylamine gave 54 mg of 6-[[(2R)-1-cyclopentyl-2-ethyl-1,2,3,4-tetrahydro-4-methyl-3-oxopyrido[3,4-b]pyrazin-7-yl]amino]-5-methoxy-N-(2,2,2-trifluoroethyl)-3-pyridinecarboxamide.

¹H NMR (300 MHz, RT, DMSO-d6): δ=0.77 (t, 3H); 1.41-1.57 (m, 1H); 1.57-1.74 (m, 3H); 1.75-1.94 (m, 4H); 1.97-2.14 (m, 2H); 3.30 (s, 3H); 3.90-4.00 (m+2, 1+3H); 4.00-4.19 (m, 3H); 7.58 (d, 1H); 7.82 (s, 1H); 7.98 (s, 1H); 8.10 (s, 1H); 8.40 (d, 1H);

### Example 8

### 6-[[(2R)-1-Cyclopentyl-2-ethyl-1,2,3,4-tetrahydro-4-methyl-3-oxopyrido[3,4-b]pyrazin-7-yl]amino]-5-methoxy-N-(2-methoxyethyl)-3-pyridinecarboxamide

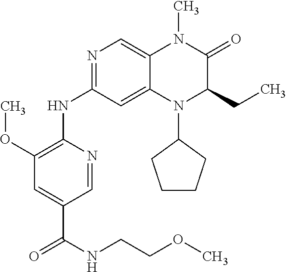 [SMILES: CC[C@@H]1C(=O)N(C)c2cnc(Nc3ncc(C(=O)NCCOC)cc3OC)cc2N1C1CCCC1]

Analogously to Example 3, 60 mg of intermediate 4.2 and 32 mg of 2-methoxyethylamine gave 50 mg of 6-[[(2R)-1-cyclopentyl-2-ethyl-1,2,3,4-tetrahydro-4-methyl-3-oxopyrido[3,4-b]pyrazin-7-yl]amino]-5-methoxy-N-(2-methoxyethyl)-3-pyridinecarboxamide.

¹H NMR (300 MHz, RT, DMSO-d6): δ=0.77 (t, 3H); 1.41-1.57 (m, 1H); 1.57-1.74 (m, 3H); 1.75-1.94 (m, 4H); 1.96-2.15 (m, 2H); 3.27 (s, 3H); 3.30 (s, 3H); 3.40-3.50 (m, 4H); 3.89-4.01 (m+s, 1+3H); 4.05 (dd, 1H); 7.65 (d, 1H); 7.81 (s, 1H); 7.91 (s, 1H); 8.10 (s, 1H); 8.34 (d, 1H); 8.55 (t, 1H);

### Example 9

### 6-{[(2R)-1-Cyclopentyl-2-ethyl-4-methyl-3-oxo-1,2,3,4-tetrahydropyrido[3,4-b]pyrazin-7-yl]amino}-5-ethyl-N-[(3R)-2-oxoazepan-3-yl]pyridine-3-carboxamide

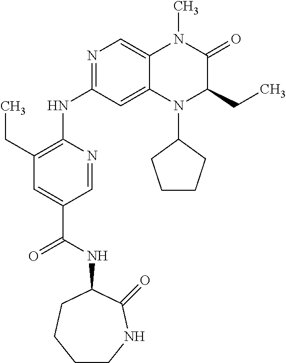 [SMILES: CCc1cc(C(=O)N[C@@H]2CCCCNC2=O)cnc1Nc1cc2c(cn1)N(C)C(=O)[C@@H](CC)N2C1CCCC1]

Analogously to Example 3, 80 mg of intermediate 2.11 and 72 mg of (R)-3-aminoazepan-2-one gave 77 mg of 6-{[(2R)-1-cyclopentyl-2-ethyl-4-methyl-3-oxo-1,2,3,4-tetrahydropyrido[3,4-b]pyrazin-7-yl]amino}-5-ethyl-N-[(3R)-2-oxoazepan-3-yl]pyridine-3-carboxamide.

¹H NMR (300 MHz, RT, DMSO-d6): δ=0.78 (t, 3H); 1.15-1.33 (m, 4H); 1.40-2.15 (m, 15H); 2.74 (q, 2H); 3.03-3.16 (m, 1H); 3.17-3.26 (m, 1H); 3.31 (s, 3H); 3.93 (qi, 1H); 4.03 (dd, 1H); 4.63 (bt, 1H); 7.78-7.87 (m, 3H); 7.93 (s, 1H); 8.23 (s, 1H); 8.29 (d, 1H); 8.56 (d, 1H);

### Example 10

### 6-[[(2R)-1-Cyclopentyl-2-ethyl-1,2,3,4-tetrahydro-4-methyl-3-oxopyrido[3,4-b]pyrazin-7-yl]amino]-5-methoxy-N-(tetrahydro-2H-pyran-4-yl)-3-pyridinecarboxamide

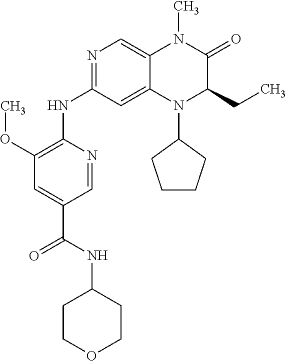 [SMILES: CC[C@@H]1C(=O)N(C)c2cnc(Nc3ncc(C(=O)NC4CCOCC4)cc3OC)cc2N1C1CCCC1]

Analogously to Example 3, 75 mg of intermediate 4.2 and 89 mg of 4-aminotetrahydro-2H-pyran gave 56 mg of 6-[[(2R)-1-cyclopentyl-2-ethyl-1,2,3,4-tetrahydro-4-methyl-3-oxopyrido[3,4-b]pyrazin-7-yl]amino]-5-methoxy-N-(tetrahydro-2H-pyran-4-yl)-3-pyridinecarboxamide.

¹H NMR (300 MHz, RT, DMSO-d6): δ=0.77 (t, 3H); 1.40-1.72 (m, 6H); 1.72-1.92 (m, 6H); 1.96-2.14 (m, 2H); 3.30 (s, 1H); 3.39 (dt, 2H); 3.89 (dd, 2H); 3.94-4.02 (m+s, 1+3H); 4.05 (dd, 1H); 7.63 (d, 1H); 7.81 (s, 1H); 7.91 (s, 1H); 8.09 (s, 1H); 8.28 (d, 1H); 8.33 (d, 1H);

### Example 11

### 6-[[(2R)-1-Cyclopentyl-2-ethyl-1,2,3,4-tetrahydro-4-methyl-3-oxopyrido[3,4-b]pyrazin-7-yl]amino]-N-(2-hydroxy-1,1-dimethylethyl)-5-methoxy-3-pyridinecarboxamide

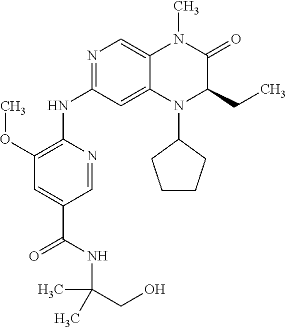 [SMILES: CC[C@@H]1C(=O)N(C)c2cnc(Nc3ncc(C(=O)NC(C)(C)CO)cc3OC)cc2N1C1CCCC1]

Analogously to Example 3, 92 mg of intermediate 4.2 and 47 mg of 2-amino-2-methylpropan-1-ol gave 65 mg of 6-[[(2R)-1-cyclopentyl-2-ethyl-1,2,3,4-tetrahydro-4-methyl-3-oxopyrido[3,4-b]pyrazin-7-yl]amino]-N-(2-hydroxy-1,1-dimethylethyl)-5-methoxy-3-pyridinecarboxamide.

¹H NMR (300 MHz, RT, DMSO-d6): δ=0.77 (t, 3H); 1.32 (s, 6H); 1.41-1.54 (m, 1H); 1.56-1.74 (m, 3H); 1.75-1.91 (m, 4H); 1.98-2.14 (m, 2H); 3.30 (s, 3H); 3.90-3.99 (m+s, 1+3H); 4.00-4.11 (m, 2H); 4.92 (bs, 1H); 7.59 (bs, 2H); 7.81 (s, 1H); 7.89 (s, 1H); 8.10 (s, 1H); 8.29 (d, 1H);

### Example 12

### 6-[[(2R)-1-Cyclopentyl-2-ethyl-1,2,3,4-tetrahydro-4-methyl-3-oxopyrido[3,4-b]pyrazin-7-yl]amino]-5-methoxy-N-(3-pyridinylmethyl)-3-pyridinecarboxamide

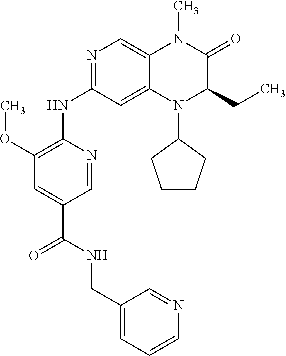 [SMILES: CC[C@@H]1C(=O)N(C)c2cnc(Nc3ncc(C(=O)NCc4cccnc4)cc3OC)cc2N1C1CCCC1]

Analogously to Example 3, 50 mg of intermediate 4.2 and 31 mg of 3-pyridylmethanamine gave 50 mg of 6-[[(2R)-1-cyclopentyl-2-ethyl-1,2,3,4-tetrahydro-4-methyl-3-oxopyrido[3,4-b]pyrazin-7-yl]amino]-5-methoxy-N-(3-pyridinylmethyl)-3-pyridinecarboxamide.

¹H NMR (300 MHz, RT, DMSO-d6): δ=0.76 (t, 3H); 1.40-1.53 (m, 1H); 1.53-1.77 (m, 3H); 1.77-1.92 (m, 4H); 1.95-2.13 (m, 2H); 3.30 (s, 3H); 3.88-4.00 (m+s, 1+3H); 4.05 (dd, 1H); 4.51 (d, 2H); 7.36 (dd, 1H); 7.67 (d, 1H); 7.73 (dt, 1H); 7.81 (s, 1H); 7.93 (s, 1H); 8.10 (s, 1H); 8.38 (d, 1H); 8.46 (dd, 1H); 8.56 (d, 1H); 9.09 (t, 1H);

### Example 13

### 6-{[(2R)-1-Cyclopentyl-2-ethyl-4-methyl-3-oxo-1,2,3,4-tetrahydropyrido[3,4-b]pyrazin-7-yl]amino}-N-cyclopropyl-5-methylpyridine-3-carboxamide

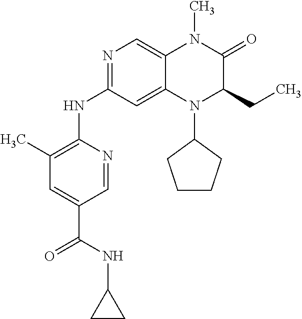 [SMILES: CC[C@@H]1C(=O)N(C)c2cnc(Nc3ncc(C(=O)NC4CC4)cc3C)cc2N1C1CCCC1]

Analogously to Example 3, 38 mg of intermediate 5.3 and 16 mg of cyclopropylamine gave 23 mg of 6-{[(2R)-1-cyclopentyl-2-ethyl-4-methyl-3-oxo-1,2,3,4-tetrahydropyrido[3,4-b]pyrazin-7-yl]amino}-N-cyclopropyl-5-methylpyridine-3-carboxamide.

¹H NMR (300 MHz, RT, DMSO-d6): δ=0.52-0.58 (m, 2H); 0.65-0.72 (m, 2H); 0.77 (t, 3H); 1.40-1.53 (m, 1H); 1.55-1.71 (m, 3H); 1.72-1.88 (m, 4H); 1.94-2.12 (m, 2H); 2.30 (s, 3H); 2.77-2.86 (dqi, 1H); 3.30 (s, 3H); 3.91 (qi, 1H); 4.03 (dd, 1H); 7.81 (s, 1H); 7.83 (s, 1H); 7.88 (d, 1H); 8.19 (s, 1H); 8.35 (d, 1H); 8.50 (d, 1H);

### Example 14

### 6-[[(2R)-1-Cyclopentyl-2-ethyl-1,2,3,4-tetrahydro-4-methyl-3-oxopyrido[3,4-b]pyrazin-7-yl]amino]-5-methoxy-N-(1-methyl-4-piperidinyl)-3-pyridinecarboxamide

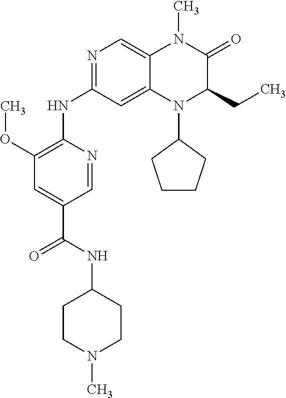 [SMILES: CC[C@@H]1C(=O)N(C)c2cnc(Nc3ncc(C(=O)NC4CCN(C)CC4)cc3OC)cc2N1C1CCCC1]

Analogously to Example 3, 74 mg of intermediate 4.2 and 95 mg of 4-amino-1-methylpiperidine gave 66 mg of 6-[[(2R)-1-cyclopentyl-2-ethyl-1,2,3,4-tetrahydro-4-methyl-3-oxopyrido[3,4-b]pyrazin-7-yl]amino]-5-methoxy-N-(1-methyl-4-piperidinyl)-3-pyridinecarboxamide.

¹H NMR (300 MHz, RT, DMSO-d6): δ=0.77 (t, 3H); 1.40-1.74 (m, 5H); 1.74-2.13 (3 m, 9H); 2.17 (s, 3H); 2.78 (bs, 2H); 3.67-3.82 (m, 1H); 3.90-4.01 (m+s, 1+3H); 4.04 (dd, 1H); 7.63 (d, 1H); 7.81 (s, 1H); 7.89 (s, 1H); 8.09 (s, 1H); 8.19 (d, 1H); 8.32 (d, 1H);

### Example 15

### 6-{[(2R)-1-Cyclopentyl-2-ethyl-4-methyl-3-oxo-1,2,3,4-tetrahydropyrido[3,4-b]pyrazin-7-yl]amino}-5-methylpyridine-3-carboxamide

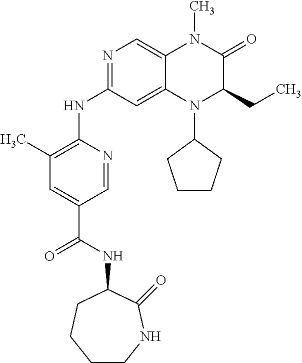 [SMILES: CC[C@@H]1C(=O)N(C)c2cnc(Nc3ncc(C(=O)N[C@@H]4CCCCNC4=O)cc3C)cc2N1C1CCCC1]

Analogously to Example 3, 38 mg of intermediate 5.3 and 36 mg of (R)-3-aminoazepan-2-one gave 32 mg of 6-{[(2R)-1-cyclopentyl-2-ethyl-4-methyl-3-oxo-1,2,3,4-tetrahydropyrido[3,4-b]pyrazin-7-yl]amino}-5-methylpyridine-3-carboxamide.

¹H NMR (300 MHz, RT, DMSO-d6): δ=0.77 (t, 3H); 1.20-1.30 (m, 1H); 1.40-1.53 (m, 1H); 1.54-1.96 (m, 12H); 1.96-2.13 (m, 2H); 2.33 (s, 3H); 3.04-3.14 (m, 1H); 3.22 (dt, 1H); 3.31 (s, 3H); 3.92 (qi, 1H); 4.03 (dd, 1H); 4.62 (dd, 1H); 7.80-7.87 (m, 3H); 7.93 (d, 1H); 8.23 (s, 1H); 8.24 (s, 1H); 8.55 (d, 1H);

### Example 16

### 6-[[(2R)-1-Cyclopentyl-2-ethyl-1,2,3,4-tetrahydro-4-methyl-3-oxopyrido[3,4-b]pyrazin-7-yl]amino]-N-[(3R)-hexahydro-2-oxo-1H-azepin-3-yl]-5-methoxy-3-pyridinecarboxamide

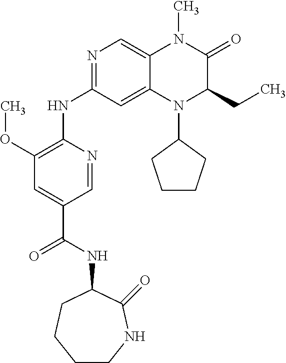 [SMILES: CC[C@@H]1C(=O)N(C)c2cnc(Nc3ncc(C(=O)N[C@@H]4CCCCNC4=O)cc3OC)cc2N1C1CCCC1]

Analogously to Example 3, 60 mg of intermediate 4.2 and 44 mg of (R)-3-aminoazepan-2-one gave 23 mg of 6-[[(2R)-1-cyclopentyl-2-ethyl-1,2,3,4-tetrahydro-4-methyl-3-oxopyrido[3,4-b]pyrazin-7-yl]amino]-N-[(3R)-hexahydro-2-oxo-1H-azepin-3-yl]-5-methoxy-3-pyridinecarboxamide.

¹H NMR (300 MHz, RT, DMSO-d6): δ=0.77 (t, 3H); 1.20-1.32 (m, 2H); 1.40-1.55 (m, 1H); 1.55-1.96 (2 m, 11H); 1.96-2.14 (m, 2H); 3.04-3.16 (m, 1H); 3.17-3.27 (m, 1H); 3.30 (s, 3H); 3.90-4.01 (m+s, 1+3H); 4.05 (dd, 1H); 4.65 (dd, 1H); 7.66 (d, 1H); 7.77-7.85 (m, 2H); 7.92 (s, 1H); 8.11 (s, 1H); 8.33-8.40 (m, 2H);

### Example 17

### 6-{[(2R)-1-Cyclopentyl-2-ethyl-4-methyl-3-oxo-1,2,3,4-tetrahydropyrido[3,4-b]pyrazin-7-yl]amino}-5-methyl-N-(tetrahydro-2H-pyran-4-yl)pyridine-3-carboxamide

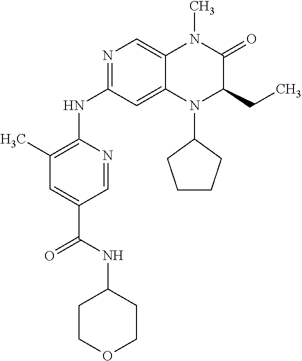 [SMILES: CC[C@@H]1C(=O)N(C)c2cnc(Nc3ncc(C(=O)NC4CCOCC4)cc3C)cc2N1C1CCCC1]

Analogously to Example 3, 35 mg of intermediate 5.3 and 26 mg of 4-aminotetrahydro-2H-pyran gave 18 mg of 6-{[(2R)-1-cyclopentyl-2-ethyl-4-methyl-3-oxo-1,2,3,4-tetrahydropyrido[3,4-b]pyrazin-7-yl]amino}-5-methyl-N-(tetrahydro-2H-pyran-4-yl)pyridine-3-carboxamide.

¹H NMR (400 MHz, RT, DMSO-d6): δ=0.78 (t, 3H); 1.41-1.53 (m, 1H); 1.53-1.70 (m, 5H); 1.71-1.87 (m, 6H); 1.94-2.12 (m, 2H); 2.32 (s, 3H); 3.31 (s, 3H); 3.38 (dt, 2H); 3.84-4.06 (m, 5H); 7.82 (s, 1H); 7.84 (s, 1H); 7.92 (d, 1H); 8.18 (s, 1H); 8.21 (d, 1H); 8.53 (d, 1H);

### Example 18

### 6-[[(2R)-1-Cyclopentyl-2-ethyl-1,2,3,4-tetrahydro-4-methyl-3-oxopyrido[3,4-b]pyrazin-7-yl]amino]-5-methyl-N-(1-methyl-4-piperidinyl)pyridine-3-carboxamide

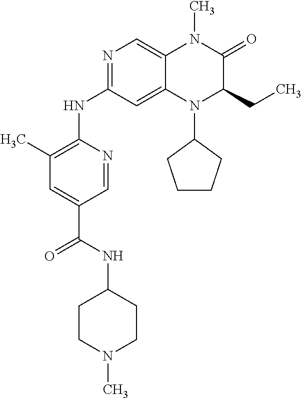 [SMILES: CC[C@@H]1C(=O)N(C)c2cnc(Nc3ncc(C(=O)NC4CCN(C)CC4)cc3C)cc2N1C1CCCC1]

Analogously to Example 3, 51 mg of intermediate 5.3 and 43 mg of 4-amino-1-methylpiperidine gave 36 mg of 6-[[(2R)-1-cyclopentyl-2-ethyl-1,2,3,4-tetrahydro-4-methyl-3-oxopyrido[3,4-b]pyrazin-7-yl]amino]-5-methyl-N-(1-methyl-4-piperidinyl)pyridine-3-carboxamide.

¹H NMR (400 MHz, RT, DMSO-d6): δ=0.77 (t, 3H); 1.40-1.52 (m, 1H); 1.52-1.69 (m, 5H); 1.71-1.86 (m, 6H); 1.92 (dt, 2H); 1.96-2.11 (m, 2H); 2.15 (s, 3H); 2.31 (s, 3H); 2.76 (bd, 2H); 3.30 (s, 3H); 3.65-3.77 (m, 1H); 3.91 (qi, 1H); 4.04 (dd, 1H); 7.81 (s, 1H); 7.83 (s, 1H); 7.91 (d, 1H); 8.15 (d, 1H); 8.19 (s, 1H); 8.52 (d, 1H);

### Example 19

### 1N-Cyclopentyl-7-[[5-(4,5-dihydro-4,4-dimethyl-2-oxazolyl)-3-methoxy-2-pyridinyl]amino]-(2R)-ethyl-4N-methyl-1,4-dihydropyrido[3,4-b]pyrazin-3(2H)-one

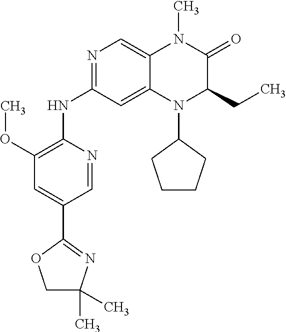 [SMILES: CC[C@@H]1C(=O)N(C)c2cnc(Nc3ncc(C4=NC(C)(C)CO4)cc3OC)cc2N1C1CCCC1]

At 0° C., 65 mg of Burgess reagent and 4.1 ml of DMF were added to a solution of 45 mg of Example 11 in 4.1 ml of THF. After 30 minutes, 66.4 mg of sodium dihydrogenphosphate were added and the mixture was stirred at room temperature for 14 hours. The mixture was stirred at 40° C. for a further 2 hours and another 18 hours at room temperature. The mixture was added to saturated sodium bicarbonate solution and extracted with ethyl acetate/methanol. The solvent was removed under reduced pressure and the residue was recrystallized from ethanol. This gave 29 mg of 1N-cyclopentyl-7-[[5-(4,5-dihydro-4,4-dimethyl-2-oxazolyl)-3-methoxy-2-pyridinyl]amino]-(2R)-ethyl-4N-methyl-1,4-dihydropyrido[3,4-b]pyrazin-3(2H)-one.

¹H NMR (400 MHz, RT, DMSO-d6): δ=0.76 (t, 3H); 1.29 (s, 6H); 1.44-1.56 (m, 1H); 1.57-1.75 (m, 3H); 1.77-1.92 (m, 4H); 1.96-2.11 (m, 2H); 3.30 (s, 1H); 3.90-4.00 (m+s, 1+3H); 4.05 (dd, 1H); 4.10 (s, 2H); 7.49 (d, 1H); 7.81 (s, 1H); 7.97 (s, 1H); 8.08 (s, 1H); 8.21 (d, 1H);

### Example 20

### N-Cyclohexyl-6-[[(2R)-1-cyclopentyl-2-ethyl-1,2,3,4-tetrahydro-4-methyl-3-oxopyrido[3,4-b]pyrazin-7-yl]amino]-5-methoxypyridine-3-carboxamide

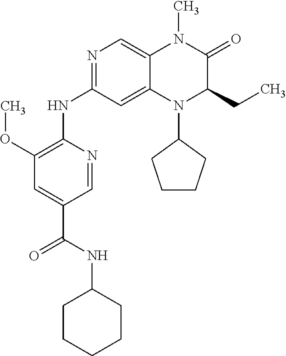 [SMILES: CC[C@@H]1C(=O)N(C)c2cnc(Nc3ncc(C(=O)NC4CCCCC4)cc3OC)cc2N1C1CCCC1]

Analogously to Example 3, 50 mg of intermediate 4.2 and 35 mg of cyclohexanamine gave 26 mg of N-cyclohexyl-6-[[(2R)-1-cyclopentyl-2-ethyl-1,2,3,4-tetrahydro-4-methyl-3-oxopyrido[3,4-b]pyrazin-7-yl]amino]-5-methoxypyridine-3-carboxamide.

¹H NMR (400 MHz, RT, DMSO-d6): δ=0.77 (t, 3H); 1.07-1.20 (m, 1H); 1.31 (qi, 4H); 1.41-1.54 (m, 1H); 1.57-1.91 (3 m, 12H); 1.97-2-13 (m, 2H); 3.30 (s, 3H); 3.78 (m, 1H); 3.91-4.00 (m+s, 1+3H); 4.04 (dd, 1H); 7.63 (d, 1H); 7.81 (s, 1H); 7.89 (s, 1H); 8.10 (s, 1H); 8.18 (d, 1H); 8.32 (d, 1H);

### Biological Efficacy of the Compounds According to the Invention

Protein-Protein Interaction Assay: BRD4/Acetylated Peptide H4 Binding Assay

### 1. Assay Description for BRD4 Bromo Domain 1 [BRD4(1)]

To assess the BRD4(1) binding strength of the substances described in this application, the ability thereof to inhibit the interaction between BRD4(1) and acetylated histone H4 in a dose-dependent manner was quantified.

For this purpose, a time-resolved fluorescence resonance energy transfer (TR-FRET) assay was used, which measures the binding between N-terminally His6-tagged BRD4(1) (amino acids 67-152) and a synthetic acetylated histone H4 (Ac-H4) peptide with sequence GRGK(Ac)GGK(Ac)GLGK(Ac)GGAK(Ac)RHGSGSK-biotin. The recombinant BRD4(1) protein produced in-house according to Filippakopoulos et al., Cell, 2012, 149:214-231 was expressed in E. coli and purified by means of (Ni-NTA) affinity and (Sephadex G-75) size exclusion chromatography. The Ac-H4 peptide can be purchased, for example, from Biosyntan (Berlin, Germany).

In the assay, typically 11 different concentrations of each substance (0.1 nM, 0.33 nM, 1.1 nM, 3.8 nM, 13 nM, 44 nM, 0.15 μM, 0.51 μM, 1.7 μM, 5.9 μM and 20 μM) were analysed as duplicates on the same microtitre plate. For this purpose, 100-fold concentrated solutions in DMSO were prepared by serial dilutions (1:3.4) of a 2 mM stock solution into a clear, 384-well microtitre plate (Greiner Bio-One, Frickenhausen, Germany). From this, 50 n1 were transferred into a black test plate (Greiner Bio-One, Frickenhausen, Germany). The test was started by the addition of 2 μl of a 2.5-fold concentrated BRD4(1) solution (final concentration typically 10 nM in the 5 μl of reaction volume) in aqueous assay buffer [50 mM HEPES pH 7.5, 50 mM sodium chloride (NaCl), 0.25 mM CHAPS and 0.05% serum albumin (BSA)] to the substances in the test plate. This was followed by a 10-minute incubation step at 22° C. for the pre-equilibration of putative complexes between BRD4(1) and the substances. Subsequently, 3 μl of a 1.67-fold concentrated solution (in assay buffer) consisting of Ac-H4 peptide (83.5 nM) and TR-FRET detection reagents [16.7 nM anti-6His-XL665 and 3.34 nM streptavidin cryptate (both from Cisbio Bioassays, Codolet, France), and 668 mM potassium fluoride (KF)] were added.

The mixture was then incubated in the dark at 22° C. for one hour and then at 4° C. for at least 3 hours and for no longer than overnight. The formation of BRD4(1)/Ac-H4 complexes was determined by the measurement of the resonance energy transfer from the streptavidin-Eu cryptate to the anti-6His-XL665 antibody present in the reaction. For this purpose, the fluorescence emission was measured at 620 nm and 665 nm after excitation at 330-350 nm in a TR-FRET measuring instrument, for example a Rubystar or Pherastar (both from BMG Lab Technologies, Offenburg, Germany) or a Viewlux (Perkin-Elmer). The ratio of the emissions at 665 nm and at 622 nm was taken as an indicator of the amount of BRD4(1)/Ac-H4 complexes formed.

The data (ratios) obtained were normalized, with 0% inhibition corresponding to the mean from the measurements for a set of controls (typically 32 data points) in which all the reagents were present. In these, in place of test substances, 50 nl of DMSO (100%) were used Inhibition of 100% corresponded to the mean from the measurements for a set of controls (typically 32 data points) in which all the reagents except BRD4(1) were present. The IC₅₀ was determined by regression analysis based on a 4-parameter equation (minimum, maximum, IC₅₀, Hill; Y=Max+(Min−Max)/(1+(X/IC₅₀)Hill).

### 2. Plk-1 Enzyme Assay

Recombinant fusion protein consisting of GST and Plk (kinase domain 33-345; MW 36 kDa, conc 0.8 μg/μl) expressed from insect cells (Hi5) and purified by glutathione Sepharose affinity chromatography and subsequent gel filtration (Superdex 75) is used for the kinase assay. Aliquots thereof are frozen in liquid nitrogen and stored at −80° C. and, after thawing, used only once.

The assay used is an indirect HTRF assay which employs the following materials and procedures. The substrate used for the kinase reaction is the biotinylated peptide Btn-Ahx-KKLNRTLSFAEPG-amide x TFA from Biosyntan, Sample No.: 6178.1 (C-terminus in amide form). This is an artificial sequence not derived from any known protein. 50 nl of the test compounds dissolved in 100% dimethyl sulphoxide (DMSO) (final concentrations: 0 μM and concentrations in the range of 0.001-20 μM) are pre-incubated with 2 μl Plk-1 enzyme working solution in working buffer [25 mM MgCl₂; 1 mM DTT; 50 mM Hepes pH 7.0; 0.01% NP40; 1× Complete; 0.05% BSA] for 30 min. The kinase reaction is then initiated by addition of 3 μl of substrate solution [adenosine triphosphate (ATP) and 1.4 μM substrate peptide (biotin-Ttds-KKLNRTLSFAEPG-NH2)] in working buffer, and, after 30 min, stopped by addition of a stopper solution (100 mM EDTA, 100 mM Hepes pH 7.5, 800 mM potassium fluoride, 0.12% BSA, 0.4 μM SA-XLent (0.05 μM, from CIS bio international, Marcoule, France), Eu³⁺ cryptate-conjugated rabbit anti-mouse IgG (1.5 nM; an anti-mouse IgG antibody labelled with europium cryptate from CIS bio international, Marcoule, France), 1 nM anti-phospho-serine kinase (a phospho-specific antibody from Upstate Biotechnology, Dundee, Scotland), and incubated at 4° C. overnight.

For the test at low ATP concentration, a 1.25 ng/μl Plk-1 working solution and 16.7 μM ATP are used, for the test at high ATP concentration, a 0.039 ng/μl Plk-1 working solution and 16.7 mM ATP are used.

The amount of phosphorylated substrate peptide is then determined by measuring the resonance energy transfer from europium-labelled antibody complex to streptavidine-XLent. To this end, the fluorescence emission at 620 nm and 665 nm is measured following excitation at 350 nm in an HTRF measuring instrument, e.g. Rubystar (BMG Labtechnologies, Offenburg, Germany) or Viewlux (Perkin-Elmer). The ratio of the emissions at 665 nm and at 620 nm is taken as a measure of the amount of phosphorylated substrate peptide. The data are normalized (enzyme reaction without inhibitor=0% inhibition, all other assay components, but no enzyme=100% inhibition), and IC₅₀ values are calculated using a 4 parameter equation (minimum, maximum, IC₅₀, Hill; Y=max+(min−max)/(1+(X/IC₅₀)Hill)).

### 4. Cell Assay

### Cell Proliferation Assay

In accordance with the invention, the substances were tested for their ability to inhibit the proliferation of the MOLM-13 cell linie (Deutsche Sammlung far Mikroorganismen and Zellkulturen [German Collection of Microorganisms and Cell Cultures], ACC 554). Cell viability was determined by means of the alamarBlue® reagent (Invitrogen) in a Victor X3 Multilabel Reader (Perkin Elmer). The excitation wavelength was 530 nm and the emission wavelength 590 nM.

The MOLM-13 cells were shown at a density of 4000 cells/well in 100 μl of growth medium on 96-well microtitre plates. After overnight incubation at 37° C., the fluorescence values (CI values) were determined. The plates were then treated with various substance dilutions and incubated at 37° C. for 96 hours. Subsequently, the fluorescence values were determined (CO values). For the data analysis, the CI values were subtracted from the CO values and the results were compared between cells which had been treated with various dilutions of the substance or only with buffer solution. The IC₅₀ values (substance concentration needed for 50% inhibition of cell proliferation) were calculated therefrom.

### 5. Results

### 5.1 Binding Assay

Table 1 shows the results from the BRD4(1) binding assay.

TABLE 1

| | IC₅₀ [BRD4(1)] |
|---|---|
| Example | (nmol/l) |
| 1 | 204 |
| 2 | 126 |
| 3 | 199 |
| 4 | 450 |
| 5 | 225 |
| 6 | 243 |
| 7 | 276 |
| 8 | 333 |
| 9 | 343 |
| 10 | 376 |
| 11 | 393 |
| 12 | 424 |
| 13 | 434 |
| 14 | 458 |
| 15 | 496 |
| 16 | 525 |
| 17 | 539 |
| 18 | 649 |
| 19 | 732 |
| 20 | 878 |

### 5.2 Kinase Activity Assay

Table 2 shows the results of the Plk-1 assays at 10 μM ATP.

TABLE 2

| | IC₅₀ [Plk-1] |
|---|---|
| | (nmol/l, 10 μM |
| Example | ATP) |
| 1 | 15 |
| 2 | 17 |
| 3 | 8 |
| 5 | 7 |
| 7 | 11 |
| 8 | 9 |
| 13 | 7 |
| 14 | 11 |
| 15 | 13 |
| 17 | 8 |

### 5.3 Kinase Activity Assay

Table 3 shows the results of the Plk-1 assays at 10 mM ATP.

TABLE 3

| | IC₅₀ [Plk-1] |
|---|---|
| | (nmol/l, 10 mM |
| Example | ATP) |
| 3 | 17 |
| 4 | 31 |
| 5 | 21 |
| 6 | 19 |
| 7 | 30 |
| 8 | 22 |
| 9 | 17 |
| 10 | 31 |
| 11 | 57 |
| 12 | 23 |
| 13 | 15 |
| 14 | 29 |
| 15 | 22 |
| 16 | 22 |
| 17 | 14 |
| 18 | 7 |
| 19 | 99 |
| 20 | 35 |

### 5.4 Cell Proliferation Assay

Table 4 shows the results from the MOLM-13 cell proliferation assay.

TABLE 4
The ability of the compounds according to the invention to inhibit the
proliferation of the MOLM-13 cell line was determined.

| | IC₅₀ (MOLM- |
|---|---|
| Example | 13) (nmol/l) |
| 1 | 209 |
| 2 | 93 |
| 3 | 61 |
| 4 | 68 |
| 5 | 29 |
| 6 | 114 |
| 7 | 80 |
| 8 | 72 |
| 9 | 76 |
| 10 | 43 |
| 11 | 74 |
| 12 | 52 |
| 13 | 56 |
| 14 | 32 |
| 15 | 77 |
| 16 | 77 |
| 17 | 55 |
| 18 | 42 |
| 19 | 362 |
| 20 | 101 |

1. A compound of formula (I) 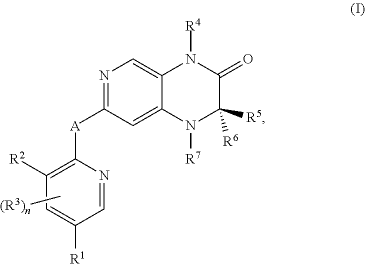
in which
A represents —NH— or —O—,
R¹ represents a —C(═O)NR⁸R⁹ or —S(═O)₂NR⁸R⁹ group, or
represents oxazolin-2-yl which may optionally be mono- or disubstituted by C₁-C₃-alkyl-, or
represents 5-membered monocyclic heteroaryl- which may optionally be mono-, di- or trisubstituted by identical or different substituents from the group
consisting of halogen, cyano, C₁-C₄-alkyl-, C₂-C₄-alkenyl-, C₂-C₄-alkynyl-, halo-C₁-C₄-alkyl-, C₁-C₄-alkoxy-, halo-C₁-C₄-alkoxy-, C₁-C₄-alkylthio-, halo-C₁-C₄-alkylthio-,
—NR¹⁰R¹¹, —C(═O)OR¹², —C(═O)N¹⁰R¹¹, —C(═O)R¹², —S(═O)₂R¹², —S(═O)₂NR¹⁰R¹¹,
R² represents hydrogen, halogen, cyano, C₁-C₄-alkyl-, C₂-C₄-alkenyl-, C₂-C₄-alkynyl-, halo-C₁-C₄-alkyl-, C₁-C₄-alkoxy-, halo-C₁-C₄-alkoxy-, C₁-C₄-alkylthio- or halo-C₁-C₄-alkylthio-,
R³ represents halogen, C₁-C₃-alkyl-, C₁-C₃-alkoxy- or cyano,
R⁴ represents methyl- or ethyl-,
R⁵ represents hydrogen or C₁-C₃-alkyl-,
R⁶ represents hydrogen or C₁-C₃-alkyl, or
R⁵ and R⁶ together with the carbon atom to which they are attached represent C₃-C₆-cycloalkyl,
R⁷ represents C₁-C₆-alkyl- which may optionally be monosubstituted by phenyl-, C₃-C₈-cycloalkyl-, or 4- to 8-membered heterocycloalkyl-,
in which phenyl- for its part may optionally be mono-, di- or trisubstituted by identical or different substituents from the group consisting of: halogen, cyano, C₁-C₄-alkyl-, C₂-C₄-alkenyl-, C₂-C₄-alkynyl-, C₁-C₄-alkoxy-, halo-C₁-C₄-alkyl-, halo-C₁-C₄-alkoxy-, and
in which C₃-C₈-cycloalkyl- and 4- to 8-membered heterocycloalkyl- for their part may optionally be mono- or disubstituted by C₁-C₃-alkyl-, or
represents C₃-C₈-cycloalkyl- or 4- to 8-membered heterocycloalkyl- which may optionally be mono- or disubstituted by C₁-C₃-alkyl-,
R⁸ represents C₁-C₆-alkyl- which may optionally be mono-, di- or trisubstituted by identical or different substituents from the group consisting of: hydroxy, oxo, fluorine, cyano, C₁-C₄-alkoxy-, halo-C₁-C₄-alkoxy-, —NR¹⁰R¹¹, C₃-C₈-cycloalkyl-, C₄-C₈-cycloalkenyl-, 4- to 8-membered heterocycloalkyl-, 4- to 8-membered heterocycloalkenyl-, C₅-C₁₁-spirocycloalkyl-, C₅-C₁₁-heterospirocycloalkyl-, bridged C₆-C₁₂-cycloalkyl-, bridged C₆-C₁₂-heterocycloalkyl-, C₆-C₁₂-bicycloalkyl-, C₆-C₁₂-heterobicycloalkyl-, phenyl- or 5- to 6-membered heteroaryl-,
in which C₃-C₈-cycloalkyl-, C₄-C₈-cycloalkenyl-, 4- to 8-membered heterocycloalkyl-, 4- to 8-membered heterocycloalkenyl-, C₅-C₁₁-spirocycloalkyl-, C₅-C₁₁-heterospirocycloalkyl-, bridged C₆-C₁₂-cycloalkyl-, bridged C₆-C₁₂-heterocycloalkyl-, C₆-C₁₂-bicycloalkyl-, C₆-C₁₂-heterobicycloalkyl- may optionally be mono- or disubstituted by identical or different substituents from the group consisting of: hydroxy, fluorine, oxo, cyano, C₁-C₃-alkyl-, fluoro-C₁-C₃-alkyl-, C₃-C₆-cycloalkyl-, cyclopropylmethyl-, C₁-C₃-alkylcarbonyl-, C₁-C₄-alkoxycarbonyl- and —NR¹⁰R¹¹, and
in which phenyl and 5- to 6-membered heteroaryl may optionally be mono- or disubstituted by identical or different substituents from the group consisting of:
halogen, cyano, trifluoromethyl-, C₁-C₃-alkyl-, C₁-C₃-alkoxy-,
or represents C₃-C₆-alkenyl or C₃-C₆-alkynyl,
or represents fluoro-C₁-C₃-alkyl- which may optionally be monosubstituted by cyano or hydroxy,
or represents C₃-C₈-cycloalkyl-, C₄-C₈-cycloalkenyl-, C₅-C₁₁-spirocycloalkyl-, bridged C₆-C₁₂-cycloalkyl- or C₆-C₁₂-bicycloalkyl- which may optionally be mono- or disubstituted by identical or different substituents from the group consisting of: hydroxy, oxo, cyano, fluorine, C₁-C₃-alkyl, C₁-C₃-alkoxy, trifluoromethyl, —NR¹⁰R¹¹,
or represents 4- to 8-membered heterocycloalkyl-, 4- to 8-membered heterocycloalkenyl-, C₅-C₁₁-heterospirocycloalkyl-, bridged C₆-C₁₂-heterocycloalkyl- or C₆-C₁₂-heterobicycloalkyl- which may optionally be mono- or disubstituted by identical or different substituents from the group consisting of: hydroxy, fluorine, oxo, cyano, C₁-C₃-alkyl-, fluoro-C₁-C₃-alkyl-, C₃-C₆-cycloalkyl-, cyclopropylmethyl-, C₁-C₃-alkylcarbonyl-, C₁-C₄-alkoxycarbonyl- and —NR¹⁰R¹¹,
R⁹ represents hydrogen or represents C₁-C₃-alkyl- which is optionally mono- or disubstituted by identical or different substituents from the group consisting of hydroxy, oxo, C₁-C₃-alkoxy-, or represents fluoro-C₁-C₃-alkyl, or
R⁸ and R⁹ together with the nitrogen atom to which they are attached represent 4- to 8-membered heterocycloalkyl, 4- to 8-membered heterocycloalkenyl-, C₅-C₁₁-heterospirocycloalkyl-, bridged C₆-C₁₂-heterocycloalkyl- or C₆-C₁₂-heterobicycloalkyl- which may optionally be mono- or disubstituted by identical or different substituents from the group consisting of: hydroxy, fluorine, oxo, cyano, C₁-C₃-alkyl-, fluoro-C₁-C₃-alkyl-, C₃-C₆-cycloalkyl-, cyclopropylmethyl-, C₁-C₃-alkylcarbonyl-, C₁-C₄-alkoxycarbonyl- and —NR¹⁰R¹¹,
R¹⁰ and R¹¹ independently of one another represent hydrogen or represent C₁-C₃-alkyl which is optionally mono- or disubstituted by identical or different substituents from the group consisting of hydroxy, oxo, C₁-C₃-alkoxy-, or represents fluoro-C₁-C₃-alkyl, or
R¹⁰ and R¹¹ together with the nitrogen atom to which they are bonded represent 4- to 8-membered heterocycloalkyl- which may optionally be mono- or disubstituted by identical or different substituents from the group consisting of: hydroxy, fluorine, oxo, cyano, C₁-C₃-alkyl-, fluoro-C₁-C₃-alkyl-, C₃-C₆-cycloalkyl-, cyclopropylmethyl-, C₁-C₃-alkylcarbonyl- and C₁-C₄-alkoxycarbonyl-,
R¹² represents C₁-C₆-alkyl- or phenyl-C₁-C₃-alkyl-, and
n represents 0 or 1,
and diastereomers, racemates, polymorphs and physiologically acceptable salts thereof.

2. A compound according to claim 1,
in which
A is —NH—,
R¹ represents a —C(═O)NR⁸R⁹ or —S(═O)₂NR⁸R⁹ group, or
represents oxazolin-2-yl which may optionally be mono- or disubstituted by C₁-C₃-alkyl-, or
represents oxazolyl-, thiazolyl-, oxadiazolyl- or thiadiazolyl- which may optionally be mono- or disubstituted by identical or different substituents from the group consisting of halogen, cyano, C₁-C₃-alkyl-, trifluoromethyl-, C₁-C₃-alkoxy-, trifluoromethoxy- and —NR¹⁰R¹¹,
R² represents hydrogen, fluorine, chlorine, cyano, methyl-, ethyl-, methoxy- or ethoxy-,
R³ represents fluorine, chlorine or methyl-,
R⁴ represents methyl-,
R⁵ represents hydrogen, methyl- or ethyl-,
R⁶ represents hydrogen, methyl- or ethyl-,
R⁷ represents C₃-C₅-alkyl-, or
represents methyl- or ethyl- which may be monosubstituted by phenyl- or 4- to 8-membered heterocycloalkyl-,
in which phenyl- for its part may optionally be mono- or disubstituted by identical or different substituents from the group consisting of: fluorine, chlorine, bromine, cyano, C₁-C₃-alkyl-, C₁-C₃-alkoxy-, trifluoromethyl-, and
in which 4- to 8-membered heterocycloalkyl- for its part may optionally be mono- or disubstituted by methyl-, or
represents C₃-C₆-cycloalkyl- or 4- to 8-membered heterocycloalkyl- which may optionally be mono- or disubstituted by methyl-,
R⁸ represents C₁-C₆-alkyl- which may optionally be mono-, di- or trisubstituted by identical or different substituents from the group consisting of: hydroxy, oxo, fluorine, cyano, C₁-C₃-alkoxy-, fluoro-C₁-C₃-alkoxy-, —NR¹⁰R¹¹, 4- to 8-membered heterocycloalkyl-, phenyl- and 5- to 6-membered heteroaryl-,
in which the 4- to 8-membered heterocycloalkyl- may optionally be monosubstituted by: hydroxy, oxo, C₁-C₃-alkyl-, fluoro-C₁-C₃-alkyl-, cyclopropyl-, cyclopropylmethyl-, acetyl- or tert-butoxycarbonyl-,
and in which phenyl and 5- to 6-membered heteroaryl may optionally be mono- or disubstituted by identical or different substituents from the group consisting of: fluorine, chlorine, cyano, trifluoromethyl-, methyl-, methoxy-,
or represents fluoro-C₁-C₃-alkyl-,
or represents C₃-C₆-cycloalkyl- which may optionally be mono- or disubstituted by identical or different substituents from the group consisting of: hydroxy, oxo, cyano, fluorine, —NR¹⁰R¹¹,
or represents 4- to 8-membered heterocycloalkyl-, C₆-C₈-heterospirocycloalkyl-, bridged C₆-C₁₀-heterocycloalkyl- or C₆-C₁₀-heterobicycloalkyl- which may optionally be mono- or disubstituted by identical or different substituents from the group consisting of: hydroxy, oxo, C₁-C₃-alkyl-, fluoro-C₁-C₃-alkyl-, cyclopropyl-, cyclopropylmethyl-, acetyl- and tert-butoxycarbonyl-,
R⁹ represents hydrogen or C₁-C₃-alkyl, or
R⁸ and R⁹ together with the nitrogen atom to which they are attached represent 4- to 8-membered heterocycloalkyl-, C₆-C₈-heterospirocycloalkyl-, bridged C₆-C₁₀-heterocycloalkyl- or C₆-C₁₀-heterobicycloalkyl-,
which may optionally be mono- or disubstituted by identical or different substituents from the group consisting of: hydroxy, oxo, C₁-C₃-alkyl-fluoro-C₁-C₃-alkyl-, cyclopropyl-, cyclopropylmethyl-, acetyl- and tert-butoxycarbonyl-,
R¹⁰ and R¹¹ independently of one another represent hydrogen or represent C₁-C₃-alkyl which is optionally monosubstituted by hydroxy or oxo or represent trifluoromethyl-, or
R¹⁰ and R¹¹ together with the nitrogen atom to which they are attached represent 4- to 7-membered heterocycloalkyl- which may optionally be mono- or disubstituted by identical or different substituents from the group consisting of: hydroxy, oxo, C₃-alkyl-fluoro-C₁-C₃-alkyl-cyclopropyl-, cyclopropylmethyl-, acetyl- and tert-butoxycarbonyl-, and
n represents 0 or 1
and diastereomers, racemates, polymorphs and physiologically acceptable salts thereof.

3. A compound according to claim 1 in which
A represents —NH—,
R¹ represents a —C(═O)NR⁸R⁹ or —S(═O)₂NR⁸R⁹ group, or
represents oxazolin-2-yl which may optionally be mono- or disubstituted by C₁-C₃-alkyl-,
R² represents hydrogen, methyl-, ethyl- or methoxy-,
R⁴ represents methyl-,
R⁵ represents methyl- or ethyl-,
R⁶ represents hydrogen,
R⁷ represents C₃-C₅-alkyl-, or
represents methyl-monosubstituted by phenyl- or 4- to 6-membered heterocycloalkyl-,
in which phenyl- for its part may optionally be mono- or disubstituted by identical or different substituents from the group consisting of: fluorine, chlorine, cyano, methyl-, methoxy-, and
in which 4- to 6-membered heterocycloalkyl- for its part may optionally be monosubstituted by methyl-, or
represents C₃-C₆-cycloalkyl- or represents 4- to 6-membered heterocycloalkyl-,
R⁸ represents C₁-C₄-alkyl- which may optionally be mono- or disubstituted by hydroxy, C₁-C₃-alkoxy-, —NR¹⁰R¹¹, 4- to 8-membered heterocycloalkyl, phenyl or 5- to 6-membered heteroaryl,
in which the 4- to 8-membered heterocycloalkyl- may optionally be monosubstituted by: oxo, C₁-C₃-alkyl, fluoro-C₁-C₃-alkyl-, cyclopropyl- or cyclopropylmethyl-,
and in which phenyl and 5- to 6-membered heteroaryl may optionally be mono- or disubstituted by identical or different substituents from the group consisting of: fluorine, chlorine, cyano, trifluoromethyl-, methyl- and methoxy-,
or represents fluoro-C₁-C₃-alkyl-,
or represents C₃-C₆-cycloalkyl- which may optionally be monosubstituted by hydroxy, fluorine or —NR¹⁰R¹¹,
or represents 4- to 8-membered heterocycloalkyl- which may optionally be mono- or disubstituted by identical or different substituents from the group consisting of: oxo, C₁-C₃-alkyl, fluoro-C₁-C₃-alkyl-, cyclopropyl- and cyclopropylmethyl-,
R⁹ represents hydrogen or methyl-, or
R⁸ and R⁹ together with the nitrogen atom to which they are attached represent 5- to 6-membered heterocycloalkyl- which may optionally be mono- or disubstituted by identical or different substituents from the group consisting of: oxo, C₁-C₃-alkyl, fluoro-C₁-C₃-alkyl-, cyclopropyl- and cyclopropylmethyl-,
R¹⁰ and R¹¹ independently of one another represent hydrogen or represent C₁-C₃-alkyl-, or
R¹⁰ and R¹¹ together with the nitrogen atom to which they are attached represent 4- to 7-membered heterocycloalkyl- which may optionally be mono- or disubstituted by identical or different substituents from the group consisting of: oxo, C₁-C₃-alkyl-, fluoro-C₁-C₃-alkyl-, cyclopropyl- and cyclopropylmethyl-, and
n represents 0,
and diastereomers, racemates, polymorphs and physiologically acceptable salts thereof.

4. A compound according to claim 1 in which
A represents —NH—,
R¹ represents a —C(═O)NR⁸R⁹ group, or
represents oxazolin-2-yl which may optionally be mono- or disubstituted by C₁-C₃-alkyl-,
R² represents methyl-, ethyl- or methoxy-,
R⁴ represents methyl-,
R⁵ represents methyl- or ethyl-,
R⁶ represents hydrogen,
R⁷ represents C₃-C₅-alkyl-, or
represents C₃-C₆-cycloalkyl,
R⁸ represents C₁-C₃-alkyl- which may optionally be monosubstituted by hydroxy, C₁-C₃-alkoxy-, phenyl- or 5- to 6-membered heteroaryl-,
in which phenyl and 5- to 6-membered heteroaryl may optionally be mono- or disubstituted by identical or different substituents from the group consisting of:
fluorine, chlorine, methyl- and methoxy-,
or represents fluoro-C₁-C₃-alkyl-,
or represents C₃-C₆-cycloalkyl,
or represents 4- to 8-membered heterocycloalkyl- which may optionally be mono- or disubstituted by identical or different substituents from the group consisting of: oxo and C₁-C₃-alkyl-,
R⁹ represents hydrogen,
n represents 0,
and diastereomers, racemates, polymorphs and physiologically acceptable salts thereof.

5. A compound according to claim 1 in which
A represents —NH—,
R¹ represents a —C(═O)NR⁸R⁹ group, or
represents oxazolin-2-yl which may optionally be mono- or disubstituted by methyl-,
R² represents methyl-, ethyl- or methoxy-,
R⁴ represents methyl-,
R⁵ represents methyl- or ethyl-,
R⁶ represents hydrogen,
R⁷ represents cyclopentyl-,
R⁸ represents C₁-C₄-alkyl- which may optionally by monosubstituted by hydroxy, methoxy- or pyridinyl-,
or represents fluoro-C₁-C₂-alkyl-,
or represents C₃-C₆-cycloalkyl,
or represents 4- to 8-membered heterocycloalkyl- which may optionally be mono- or disubstituted by identical or different substituents from the group consisting of: oxo and C₁-C₃-alkyl-,
R⁹ represents hydrogen,
n represents 0,
and diastereomers, racemates, polymorphs and physiologically acceptable salts thereof.

6. A compound according to claim 1 in which
A represents —NH—,
R¹ represents a —C(═O)NR⁸R⁹ group, or
represents oxazolin-2-yl- which is disubstituted by methyl-,
R² represents methyl-, ethyl- or methoxy-,
R⁴ represents methyl-,
R⁵ represents methyl- or ethyl-,
R⁶ represents hydrogen,
R⁷ represents cyclopentyl-,
R⁸ represents C₁-C₄-alkyl- which may optionally by monosubstituted by hydroxy, methoxy- or pyridinyl-,
or represents 2,2,2-trifluoroethyl-,
or represents cyclopropyl- or cyclohexyl-,
or represents piperidinyl, azepanyl or tetrahydropyranyl which may optionally be mono- or disubstituted by identical or different substituents from the group consisting of: oxo and methyl,
R⁹ represents hydrogen,
n represents 0,
and diastereomers, racemates, polymorphs and physiologically acceptable salts thereof.

7. A compound according to claim 1, selected from the group consisting of
6-{[1-cyclopentyl-2,4-dimethyl-3-oxo-1,2,3,4-tetrahydropyrido[3,4-b]pyrazin-7-yl]amino}-N-cyclopropyl-5-methoxypyridine-3-carboxamide,
6-{[(2R)-1-cyclopentyl-2,4-dimethyl-3-oxo-1,2,3,4-tetrahydropyrido[3,4-b]pyrazin-7-yl]amino}-N-cyclopropyl-5-methoxypyridine-3-carboxamide,
6-{[(2R)-1-cyclopentyl-2-ethyl-4-methyl-3-oxo-1,2,3,4-tetrahydropyrido[3,4-b]pyrazin-7-yl]amino}-N-cyclopropyl-5-ethylpyridine-3-carboxamide,
6-[(1-cyclopentyl-2-ethyl-1,2,3,4-tetrahydro-4-methyl-3-oxopyrido[3,4-b]pyrazin-7-yl)amino]-N-cyclopropyl-5-methoxy-3-pyridinecarboxamide,
6-[[(2R)-1-cyclopentyl-2-ethyl-1,2,3,4-tetrahydro-4-methyl-3-oxopyrido[3,4-b]pyrazin-7-yl)amino]-N-cyclopropyl-5-methoxy-3-pyridinecarboxamide,
6-[(1-cyclopentyl-2,4-dimethyl-3-oxo-1,2,3,4-tetrahydropyrido[3,4-b]pyrazin-7-yl)amino]-5-methoxy-N-(1-methylpiperidin-4-yl)pyridine-3-carboxamide,
6-[[(2R)-1-cyclopentyl-2-ethyl-1,2,3,4-tetrahydro-4-methyl-3-oxopyrido[3,4-b]pyrazin-7-yl]amino]-5-methoxy-N-(2,2,2-trifluoroethyl)-3-pyridinecarboxamide,
6-[[(2R)-1-cyclopentyl-2-ethyl-1,2,3,4-tetrahydro-4-methyl-3-oxopyrido[3,4-b]pyrazin-7-yl]amino]-5-methoxy-N-(2-methoxyethyl)-3-pyridinecarboxamide,
6-{[(2R)-1-cyclopentyl-2-ethyl-4-methyl-3-oxo-1,2,3,4-tetrahydropyrido[3,4-b]pyrazin-7-yl]amino}-5-ethyl-N-[(3R)-2-oxoazepan-3-yl]pyridine-3-carboxamide,
6-[[(2R)-1-cyclopentyl-2-ethyl-1,2,3,4-tetrahydro-4-methyl-3-oxopyrido[3,4-b]pyrazin-7-yl]amino]-5-methoxy-N-(tetrahydro-2H-pyran-4-yl)-3-pyridinecarboxamide,
6-[[(2R)-1-cyclopentyl-2-ethyl-1,2,3,4-tetrahydro-4-methyl-3-oxopyrido[3,4-b]pyrazin-7-yl]amino]-N-(2-hydroxy-1,1-dimethylethyl)-5-methoxy-3-pyridinecarboxamide,
6-[[(2R)-1-cyclopentyl-2-ethyl-1,2,3,4-tetrahydro-4-methyl-3-oxopyrido[3,4-b]pyrazin-7-yl]amino]-5-methoxy-N-(3-pyridinylmethyl)-3-pyridinecarboxamide,
6-{[(2R)-1-cyclopentyl-2-ethyl-4-methyl-3-oxo-1,2,3,4-tetrahydropyrido[3,4-b]pyrazin-7-yl]amino}-N-cyclopropyl-5-methylpyridine-3-carboxamide,
6-[[(2R)-1-cyclopentyl-2-ethyl-1,2,3,4-tetrahydro-4-methyl-3-oxopyrido[3,4-b]pyrazin-7-yl]amino]-5-methoxy-N-(1-methyl-4-piperidinyl)-3-pyridinecarboxamide,
6-{[(2R)-1-cyclopentyl-2-ethyl-4-methyl-3-oxo-1,2,3,4-tetrahydropyrido[3,4-b]pyrazin-7-yl]amino}-5-methylpyridine-3-carboxamide,
6-[[(2R)-1-cyclopentyl-2-ethyl-1,2,3,4-tetrahydro-4-methyl-3-oxopyrido[3,4-b]pyrazin-7-yl]amino]-N-[(3R)-hexahydro-2-oxo-1H-azepin-3-yl]-5-methoxy-3-pyridinecarboxamide,
6-{[(2R)-1-cyclopentyl-2-ethyl-4-methyl-3-oxo-1,2,3,4-tetrahydropyrido[3,4-b]pyrazin-7-yl]amino}-5-methyl-N-(tetrahydro-2H-pyran-4-yl)pyridine-3-carboxamide,
6-[[(2R)-1-cyclopentyl-2-ethyl-1,2,3,4-tetrahydro-4-methyl-3-oxopyrido[3,4-b]pyrazin-7-yl]amino]-5-methyl-N-(1-methyl-4-piperidinyl)pyridine-3-carboxamide,
1N-cyclopentyl-7-[[5-(4,5-dihydro-4,4-dimethyl-2-oxazolyl)-3-methoxy-2-pyridinyl]amino]-(2R)-ethyl-4N-methyl-1,4-dihydropyrido[3,4-b]pyrazin-3(2H)-one, and
N-cyclohexyl-6-[[(2R)-1-cyclopentyl-2-ethyl-1,2,3,4-tetrahydro-4-methyl-3-oxopyrido[3,4-b]pyrazin-7-yl]amino]-5-methoxypyridine-3-carboxamide.

8. (canceled)

9. (canceled)

10. (canceled)

11. (canceled)

12. (canceled)

13. (canceled)

14. (canceled)

15. A compound according to claim 1 in combination with one or more further pharmacologically active substances.

16. (canceled)

17. (canceled)

18. (canceled)

19. A compound of formula (VIII) 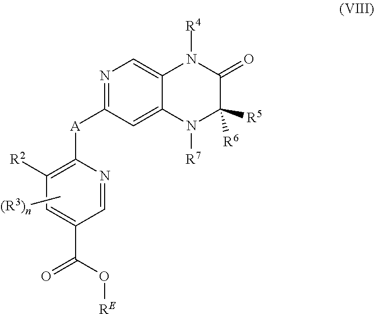
in which A, R², R³, R⁴, R⁵, R⁶, R⁷ and n have the meanings given in claim 1 and R^{E} represents C₁-C₆-alkyl.

20. A compound of formula (IX) 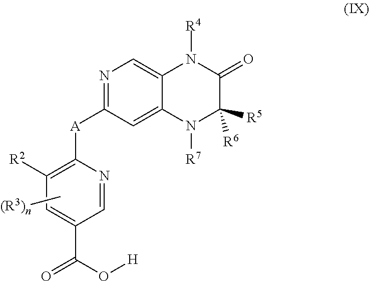
in which A, R², R³, R⁴, R⁵, R⁶, R⁷ and n have the meanings given in claim 1.

21. A method for the treatment of a neoplastic disorder comprising administering to a patient in need thereof an effective amount of a compound according to claim 1.

22. A method for the treatment of a hyperproliferative disorder comprising administering to a patient in need thereof an effective amount of a compound according to claim 1.

23. A method for the treatment of a viral infection, neurodegenerative disorder, inflammatory disorder, or atherosclerotic disorder or for male fertility control comprising administering to a patient in need thereof an effective amount of a compound according to claim 1.


SEQUENCE LISTING (3 sequences)

SEQ ID NO: 1 - protein, 22 aa, Artificial sequence
  Synthetically acetylated Histon H4 (Ac-H4) Peptide; acetylated at Lysin in position 4, 7, 11 and 15
GRGKGGKGLGKGGAKRHGSGSK

SEQ ID NO: 2 - protein, 13 aa, Artificial sequence
  Biotinylated peptide Btn-Ahx
KKLNRTLSFAEPG

SEQ ID NO: 3 - protein, 13 aa, Artificial sequence
  Biotinylated peptide Ttds
KKLNRTLSFAEPG